(19) United States
(12) Patent Application Publication
(10) Pub. No.: US 2025/0205372 A1
(43) Pub. Date: Jun. 26, 2025

(54) RADIOPHARMACEUTICAL COMPOSITIONS, SYNTHETIC METHODS, AND METHODS OF TREATMENT

(71) Applicant: The Research Foundation for The State University of New York, Albany, NY (US)

(72) Inventors: Eszter Boros, Stony Brook, NY (US); Dariusz Smilowicz, Stony Brook, NY (US)

(73) Assignee: The Research Foundation for The State University of New York, Albany, NY (US)

(21) Appl. No.: 18/848,497

(22) PCT Filed: Mar. 17, 2023
(86) PCT No.: PCT/US2023/064637

§ 371 Date: Sep. 18, 2024

Related U.S. Application Data
(60) Provisional application No. 63/321,286, filed on Mar. 18, 2022.

(51) Int. Cl. A61K 51/08

(52) U.S. Cl. CPC A61K 51/088; A61K 2121/00; A61K 2123/00

(57) ABSTRACT

The present invention provides processes of producing the following compound:

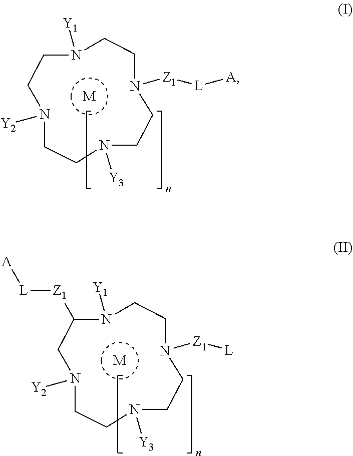

and methods of using the compound in targeted PET and SPECT imaging.

This application claims priority of U.S. Provisional Application No. 63/321,286, filed Mar. 18, 2022, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Radiopharmaceuticals enable non-invasive patient diagnosis and development of personalized treatment strategies for a vast number of clinical patients globally. Consequently, there is growing demand to develop scalable, versatile, rapid, high-yielding chemical syntheses of radiopharmaceuticals. Due to the often-defined chemical nature of radiosynthesis precursors and short isotope half-life of the radioisotope, radiochemical synthesis needs to be a fast, simple and modular, while producing the target radiopharmaceutical in high chemical yield, purity and molar activity. Radioactive isotopes for nuclear medicine applications require purification following their nuclear synthesis to separate them from the target material before they can be employed in radiosynthesis. It also requires purification prior to dose preparation (Lampkin, 2021). Evidently, this is a time-consuming, multi-step process that results in decay of a significant fraction of the radioactive isotope prior to its use in the patient.

Therefore, there is a need for radiosynthesis strategies that could condense multiple steps into a simple, timesaving one-pot procedure. Specifically, isotope capture, separation, and tracer synthesis should feasibly occur at once, followed by efficient release of the target radiopharmaceutical in a ready-to-inject form. This is inherently difficult to implement, especially for the radiolabeling with metal-ions and metalloids, which are usually separated under highly acidic conditions (2-12M HCl) from their parent nuclide using ion exchange chromatography, but subsequently require much milder, aqueous radiosynthesis conditions; however, the pH dependent speciation and chemical stability of bifunctional chelators, peptides and biomolecules utilized as disease-specific targeting vectors typically requires a pH range of 4-8.

Therefore, this invention provides an immobilization of a radiopharmaceutical synthesis precursor comprised a chelate linked peptide on water-compatible, conventional peptide resin, appended by a photo-labile resin attachment. Photochemical radiosynthesis strategies are emerging as a versatile radiosynthesis tool as they produce no significant in-solution impurities or waste materials and are compatible with high-throughput and automation procedures prevalent in the routine production of radiopharmaceuticals. Indeed, photochemical radiosynthesis has previously enabled bond-formation strategies that produce radiofluorinated arenes using photoredox catalysis, photoreactive substrate mediated substitution with radiofluoroalkenes and photochemical protein ligation of radiometal chelates.

### SUMMARY OF THE INVENTION

The present invention provides a process for preparing a compound of formula I or a compound of formula II, or a pharmaceutically acceptable salt of the compound of formula I or the compound of formula II:

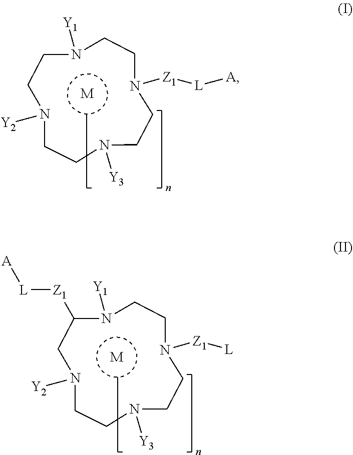

wherein
A is a targeting moiety, or an activating group or absent;
L is a peptide linker;
M is a metal that is either absent or present;
n is 0 or 1;
Y₁, Y₂, Y₃ are each, independently, —H, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ and alkylheteroaryl-P(O)(OH)₂, and
wherein each occurrence of R₄ is independently, —H, alkyl, alkenyl, alkynyl, alkyl-aryl, alkyl-heteroaryl, aryl, heteroaryl, alkyl-CF₃ or —Si(alkyl)₃;
Z₁ is

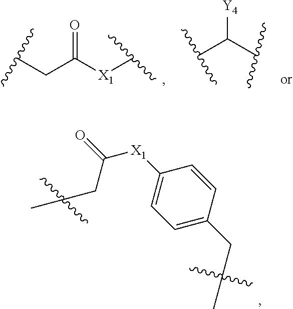

wherein X₁ is NH, O or S, and
Y₄ is —CO₂H, —CO₂R₅, aryl-CO₂H, heteroaryl-CO₂H, aryl-CO₂R₅ or heteroaryl-CO₂R₅, and
   wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
   wherein if the process is for producing compound of formula I when M is not present, then the process comprises:
(a) preparing a compound of formula I-A,

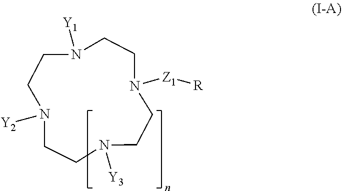

and
(b) cleaving the compound of formula I-A or the compound of II-A off a solid support surface;
   wherein if the process is for producing compound of formula I when M is present, then the process comprises:
(a) preparing a compound of formula I-A,

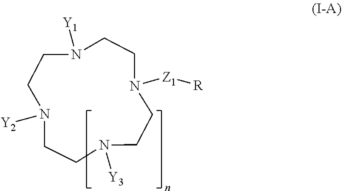

(b) complexing a metal ion with the compound of formula I-A, and
(c) cleaving the compound of formula I-A off a solid support surface;
   wherein if the process is for producing compound of formula II when M is not present, then the process comprises:
(a) preparing a compound of formula II-A,

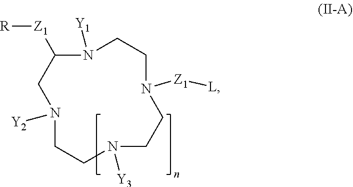

and
(b) cleaving the compound of formula II-A off a solid support surface;
   wherein if the process is for producing compound of formula I when M is present, then the process comprises:
(a) preparing a compound of formula II-A,

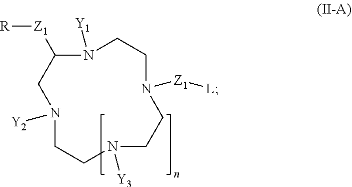

(b) complexing a metal ion with the compound of formula I-A, and
(c) cleaving the compound of formula II-A off a solid support surface;
   wherein R is

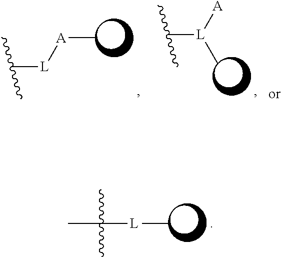

The present invention provides a composition comprising a compound attached to a solid surface having the structure:

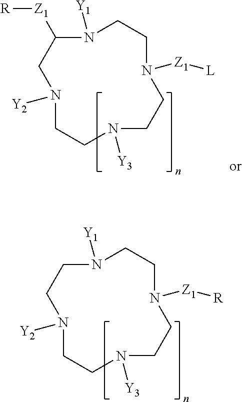

wherein
wherein R is or

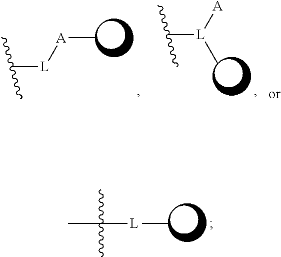

n is 0 or 1;
Y₁, Y₂, Y₃ are each, independently, —H, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ and alkylheteroaryl-P(O)(OH)₂;
wherein each occurrence of R₄ is independently, —H, alkyl, alkenyl, alkynyl, alkyl-aryl, alkyl-heteroaryl, aryl, heteroaryl, alkyl-CF₃ or —Si(alkyl)₃;
Z₁ is

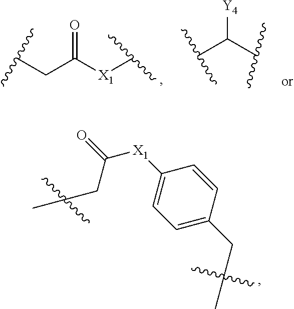

wherein X₁ is NH, O or S, and
Y₄ is —CO₂H, —CO₂R₅, aryl-CO₂H, heteroaryl-CO₂H, aryl-CO₂R₅ or heteroaryl-CO₂R₅, and
wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
   L is a peptide linker;
   A is a targeting moiety or an activating group;
or a pharmaceutically acceptable salt of the compound.

The present invention provides a method of detecting target cells in a subject comprising administering an effective amount of the metal complex described in the present invention to the subject, and imaging the subject with a molecular imaging device to detect the metal complex or composition in the subject.

The present invention provides a method of imaging target cells in a subject comprising:
a) administering to the subject an effective amount of the metal complex described in the present invention or a pharmaceutically acceptable salt thereof, wherein the metal complex or the composition specifically accumulates at the target cells in the subject;
b) detecting in the subject the location of the metal complex or the composition; and
c) obtaining an image of the target cells in the subject based on the location of the metal complex or the composition in the subject.

The present invention provides a method of detecting the presence of target cells in a subject which comprises determining if an amount of the metal complex described in the present invention or a pharmaceutically acceptable salt thereof, is present in the subject at a period of time after administration of the metal complex or composition to the subject, thereby detecting the presence of the target cells based on the amount of the metal complex or composition determined to be present in the subject.

The present invention provides a method of detecting cancer cells in a subject comprising administering an effective amount of the metal complex described in the present invention to the subject, and imaging the subject with a molecular imaging device to detect the metal complex or composition in the subject, wherein the cancer cells are prostate cancer cells, wherein the cancer cells have elevated levels of prostate-specific membrane antigen (PSMA).

The present invention provides a method of imaging prostate cancer cells in a subject comprising:
a) administering to the subject an effective amount of the metal described in the present invention or a pharmaceutically acceptable salt thereof, wherein the metal complex or the composition specifically accumulates at prostate cancer cells in the subject;
b) detecting in the subject the location of the metal complex or the composition; and
c) obtaining an image of the cancer cells in the subject based on the location of the metal complex or the composition in the subject.

The present invention provides a method of detecting the presence of prostate cancer cells in a subject which comprises determining if an amount of the metal complex described in the present invention or a pharmaceutically acceptable salt thereof, is present in the subject at a period of time after administration of the metal complex or composition to the subject, thereby detecting the presence of the prostate cancer cells based on the amount of the metal complex or composition determined to be present in the subject.

The present invention provides a method of reducing the size of a prostate tumor or of inhibiting proliferation of prostate cancer cells comprising contacting the tumor or cancer cells with the metal complex described in the present invention or a pharmaceutically acceptable salt thereof, so as to thereby reducing the size of the tumor or inhibit proliferation of the cancer cells.

### BRIEF DESCRIPTION OF THE FIGURES

FIG. 1. Photo-triggered radiometallation. (A) Prior studies rely on multi-steps radiopharmaceuticals preparation. (B) This invention obviates the need for purification by using solid phase approach.

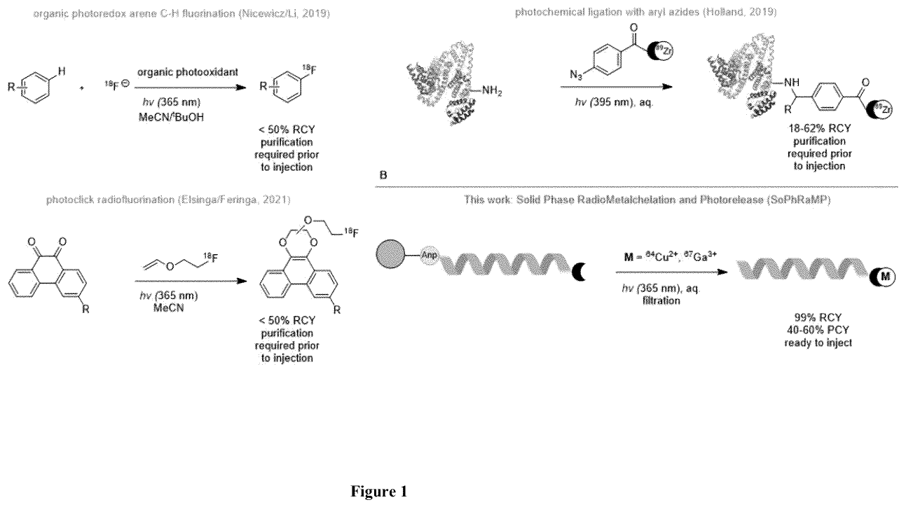

FIG. 2. (A) Reaction scheme of prototype solid phase model construct designed for ^{67/68}Ga isotopes, ⁶⁷Ga(NOTA)-Q-W-NH₂ model peptide using 3 different resin material approaches (ion exchange, agarose and tentagel). (B) Analytical radio HPLC chromatogram of crude filtrate following photocleavage showing >99% purity of the desired product with all resin materials tested. (C/D) Radiochemical and photocleavage yields show best performance for large volumes with tentagel resin material shows robust radiolabeling performance for the resin-based method with respect to radiolabeling yield using small quantities of resin (10 nmol peptide) (E) Reduction of LED/photon source to reaction vessel containing resin demonstrates vastly improved photocleavage yields for 10 min photocleavage. RCY: Radiochemical Yield. PCY: Photochemical cleavage yield.

FIG. 3. (A) Reaction scheme of SoPhRaMP construct designed for ⁶⁴Cu/⁶⁷Ga isotopes, NOTA-Aca-BBN-NH₂ peptide using single-resin strategy. (B) Analytical radio-HPLC chromatogram of crude filtrates following photocleavage showing >99% purity of the desired products. (C) Radiochemical and photocleavage yields shows robust radiolabeling performance for the resin-based method with respect to radiolabeling yield using small quantities of resin (10 nmol peptide). (D) Activity-dependent radiochemical and photocleavage yields. (E) Radiochemical yields show the best performance for large volumes for the resin-based method in comparison to radiolabeling in solution. RCY: Radiochemical Yield. PCY: Photochemical cleavage yield.

FIG. 4. Synthesis of NOTA-Aca-Gln-Trp-Anp (10).

FIG. 5. Synthesis of NOTA-Aca-BBN (19) on TENTAGEL resin.

FIG. 6. Synthesis of NOTA-PSMA (31) on TENTAGEL resin.

FIG. 7. Synthesis of Isotopically Stable Complexes.

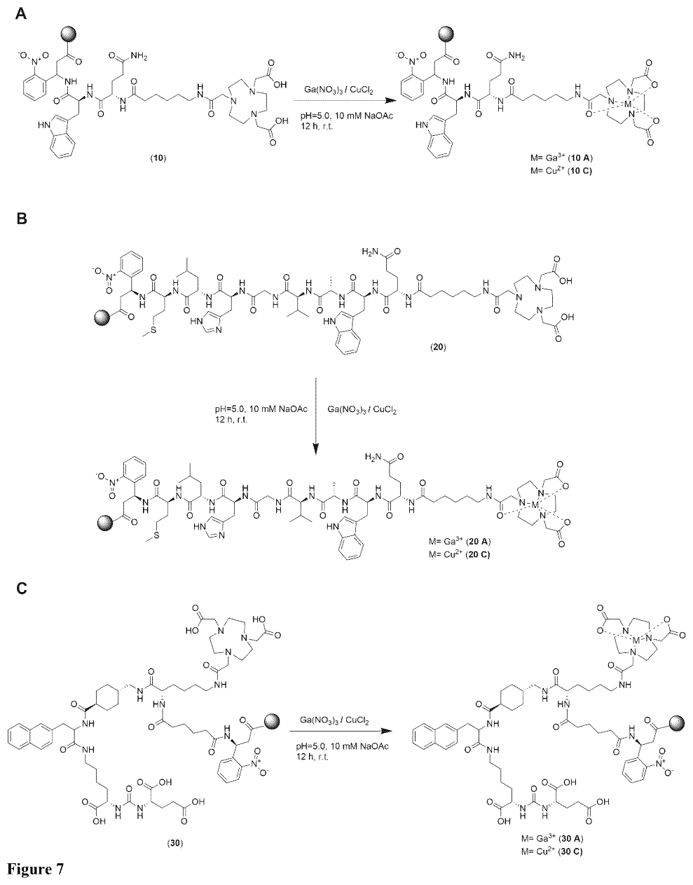

FIG. 8. Reaction scheme and corresponding radio-HPLC trace.

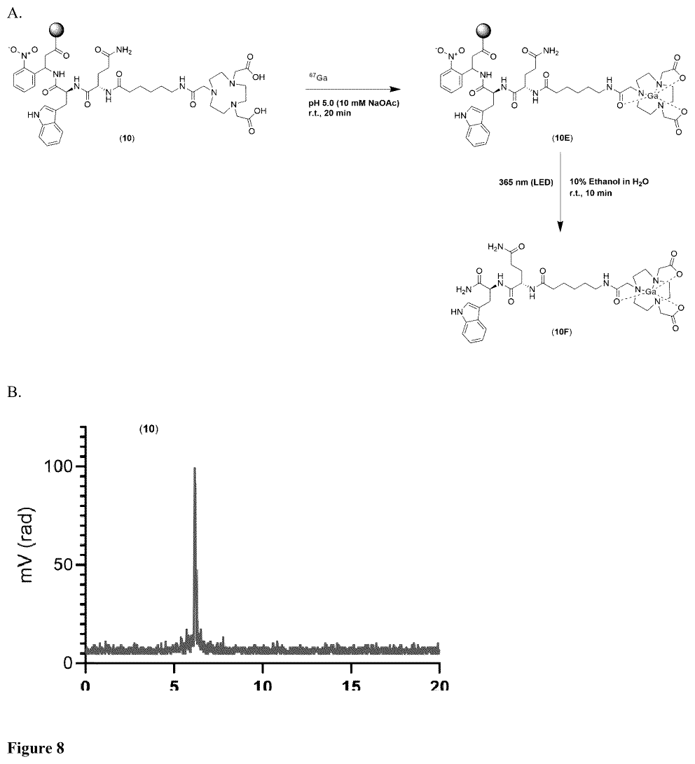

FIG. 9. Reaction scheme and corresponding radio-HPLC trace.

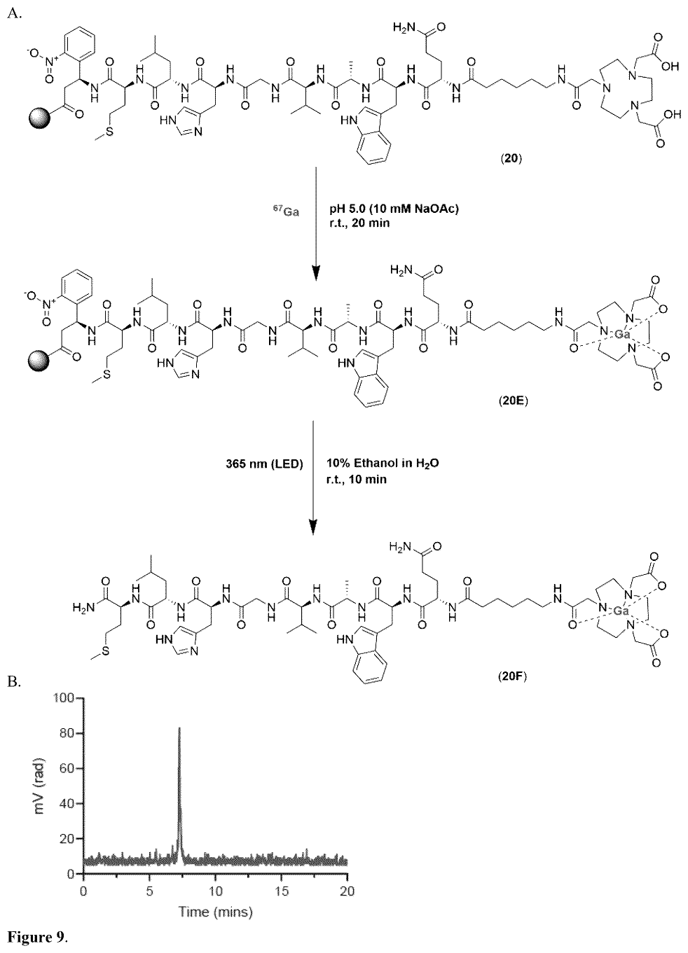

FIG. 10. Reaction scheme and corresponding radio-HPLC trace.

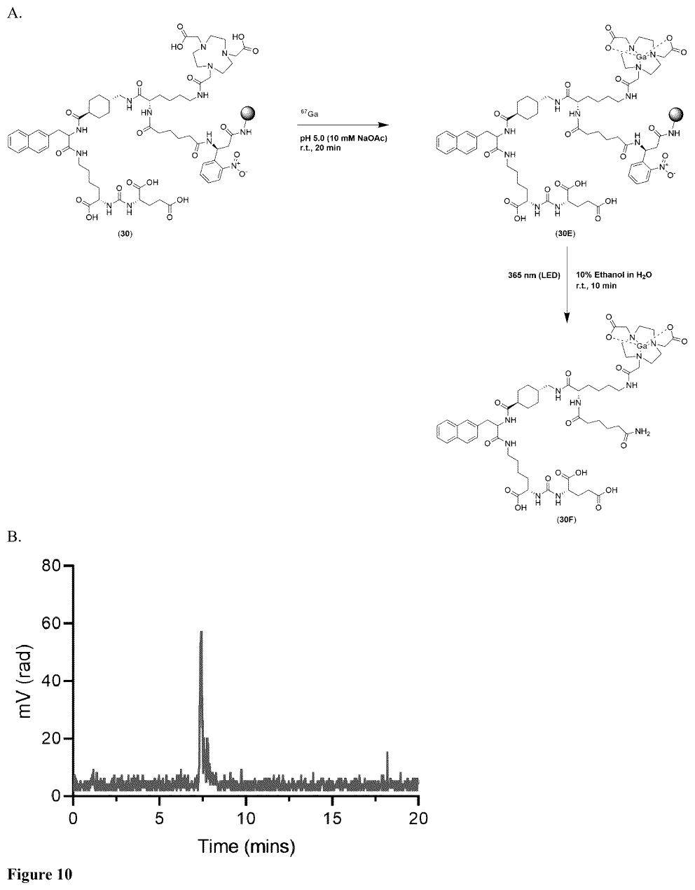

FIG. 11. Reaction scheme and corresponding radio-HPLC trace.

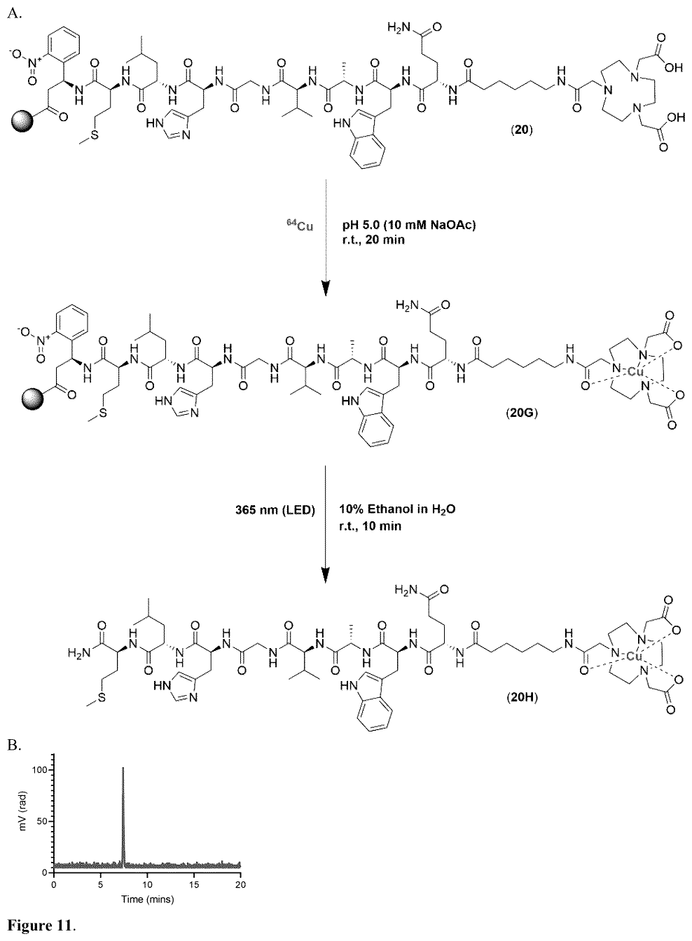

FIG. 12. Reaction scheme and corresponding radio-HPLC trace.

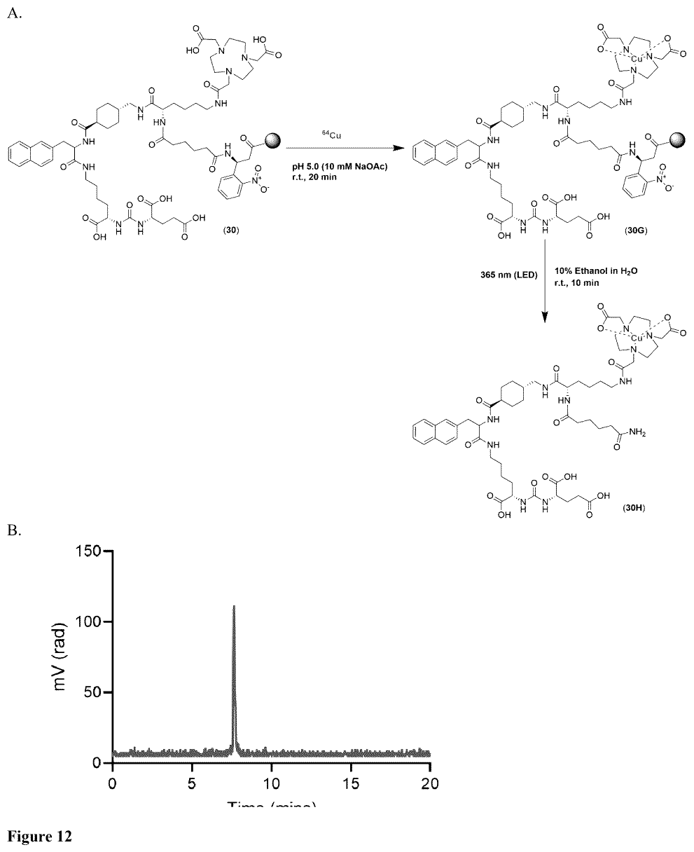

FIG. 13. Synthesis pathway of compound 3, 4, 4* and 5.

FIG. 14. Synthesis pathway of PSMA derivatives.

FIG. 15. (A) Schematic description of incubation of ligand 3. (B) Analytical HPLC chromatograms showing no autolytic release of NOTA molecule. (C) Quantification of the pH-dependent cleavage at 80° C. and pH 4.5.

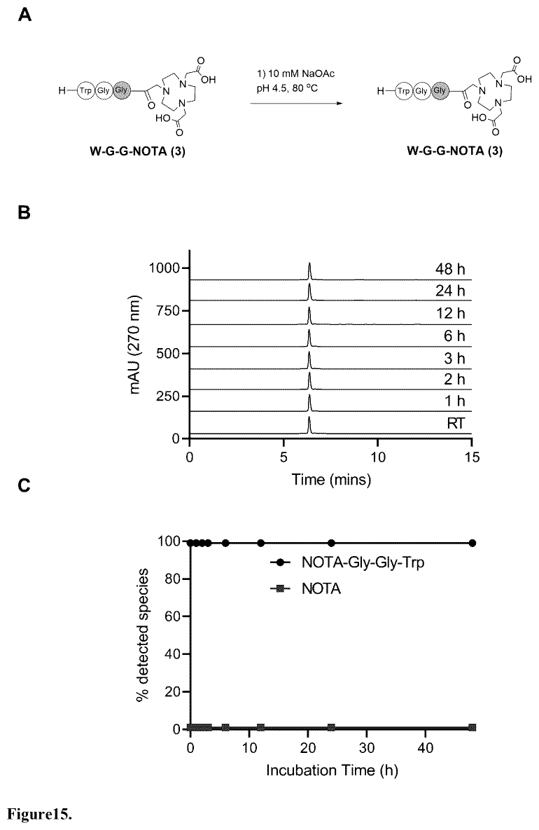

FIG. 16. Complexation pathway for ^{nat}Ga-3, ^{nat}Cu-3, ^{nat}Sc-3, ^{nat}Ga-4, ^{nat}Cu-4, ^{nat}Sc-4, ^{nat}Ga-5, ^{nat}Cu-5, and ^{nat}Sc-5.

FIG. 17. (A) Schematic description of preparation and direct complexation procedure of ^{nat}Ga-4. (B) Analytical HPLC chromatograms showing autolytic release of the desired product ^{nat}Ga-NOTA. (C) Quantification of the pH-dependent cleavage at 80° C. and pH 4.5.

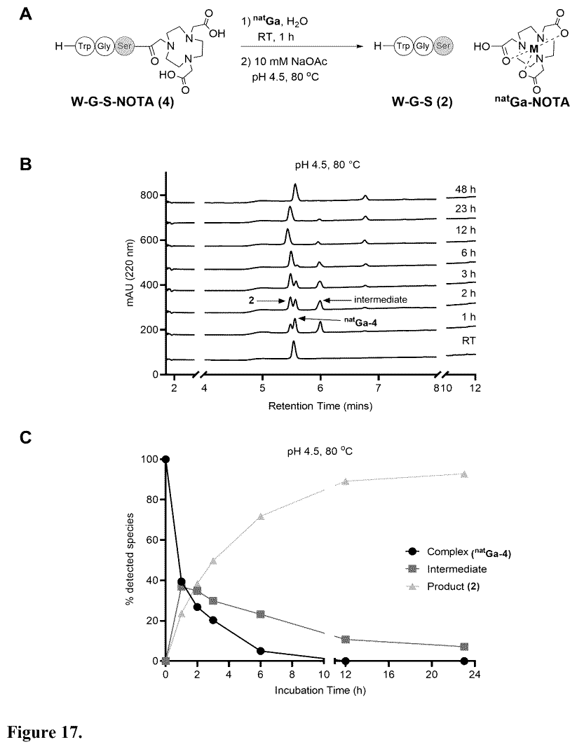

FIG. 18. (A) Schematic description of preparation and direct complexation procedure of ^{nat}Ga-4. (B) Analytical HPLC chromatograms showing autolytic release of the desired product ^{nat}Ga-NOTA. (C) Quantification of the pH-dependent cleavage at 80° C. and pH 5.5.

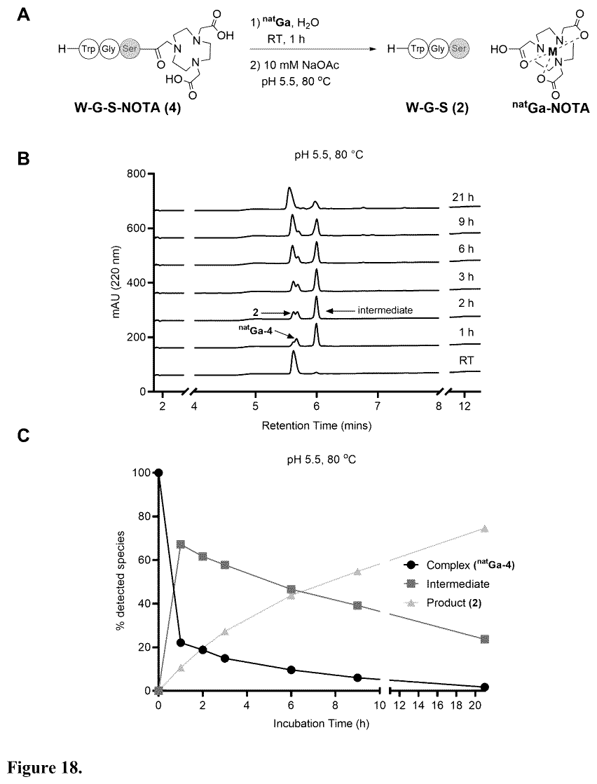

FIG. 19 (A) Schematic description of preparation and direct complexation procedure of ^{nat}Ga-4. (B) Analytical HPLC chromatograms showing autolytic release of the desired product ^{nat}Ga-NOTA. (C) Quantification of the pH-dependent cleavage at 80° C. and pH 6.5.

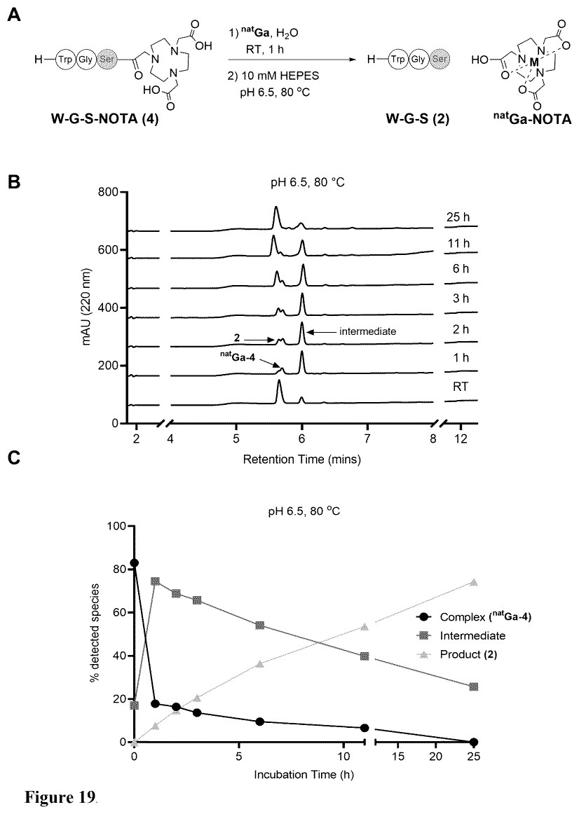

FIG. 20(A) Schematic description of preparation and direct complexation procedure of ^{nat}Ga-4. (B) Analytical HPLC chromatograms showing autolytic release of the desired product ^{nat}Ga-NOTA. (C) Quantification of the pH-dependent cleavage at 80° C. and pH 7.5.

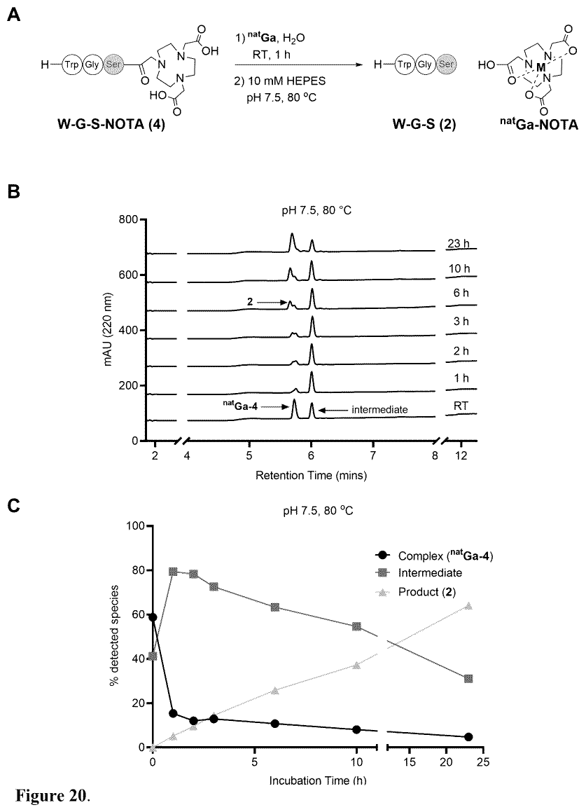

FIG. 21. (A) Schematic description of preparation and direct complexation procedure of ^{nat}Ga-4. (B) Analytical HPLC chromatograms showing autolytic release of the desired product ^{nat}Ga-NOTA. (C) Quantification of the pH-dependent cleavage at 37° C. and pH 4.5.

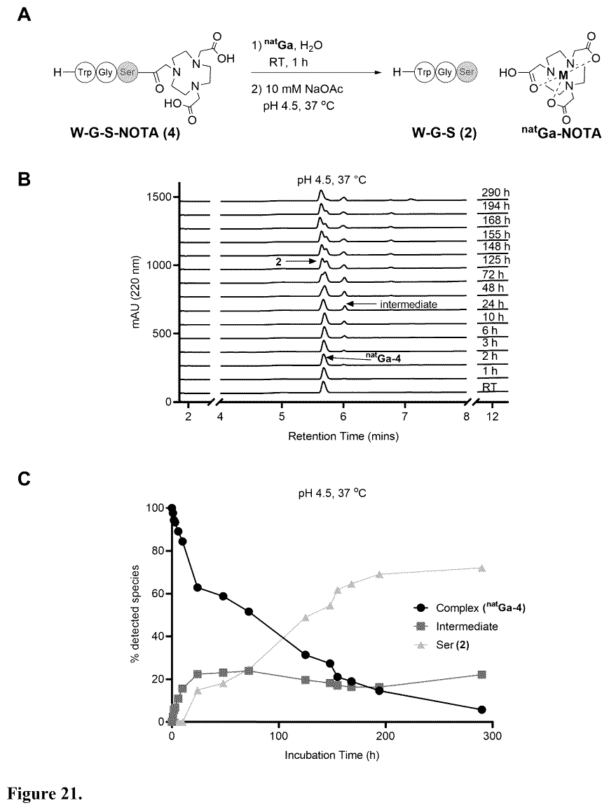

FIG. 22 (A) Schematic description of preparation and direct complexation procedure of ^{nat}Ga-5. (B) Analytical HPLC chromatograms showing no autolytic release of the ^{nat}Ga-DOTA. (C) Quantification of the pH-dependent cleavage at 80° C. and pH 4.5.

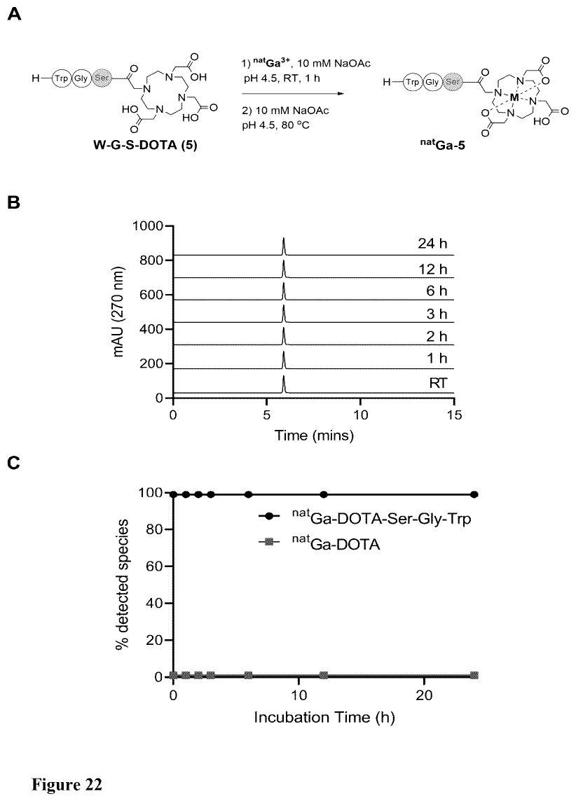

FIG. 23. (A) Schematic description of preparation and direct complexation procedure of ^{nat}Cu-3. (B) Analytical HPLC chromatograms showing no autolytic release of the ^{nat}Cu-NOTA. (C) Quantification of the pH-dependent cleavage at 80° C. and pH 4.5.

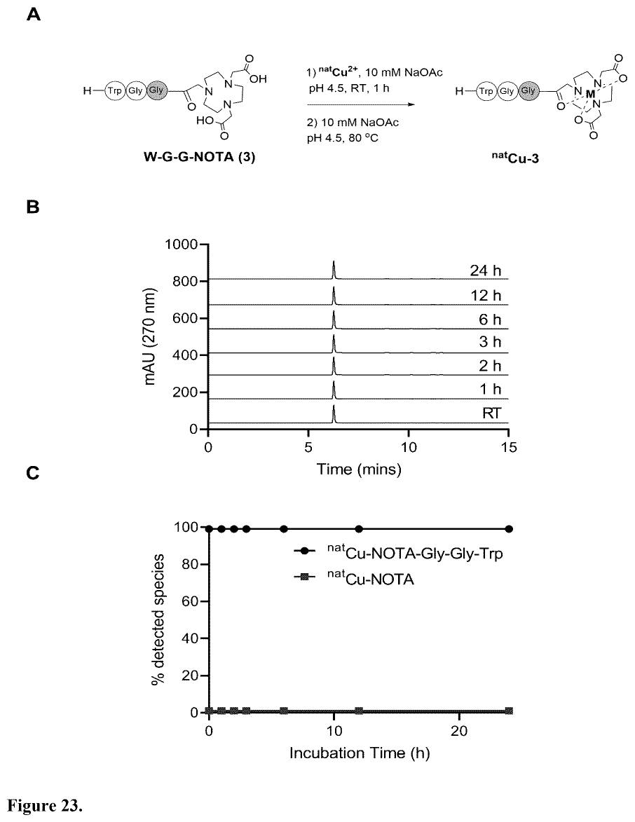

FIG. 24. (A) Schematic description of preparation and direct complexation procedure of ^{nat}Cu-4. (B) Analytical HPLC chromatograms showing no autolytic release of the ^{nat}Cu-NOTA. (C) Quantification of the pH-dependent cleavage at 80° C. and pH 4.5.

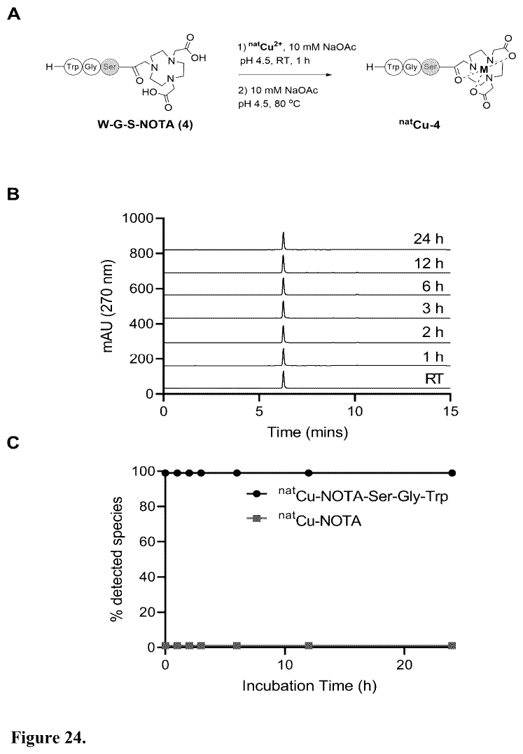

FIG. 25. (A) Schematic description of preparation and direct complexation procedure of ^{nat}Cu-5. (B) Analytical HPLC chromatograms showing no autolytic release of the ^{nat}Cu-DOTA. (C) Quantification of the pH-dependent cleavage at 80° C. and pH 4.5.

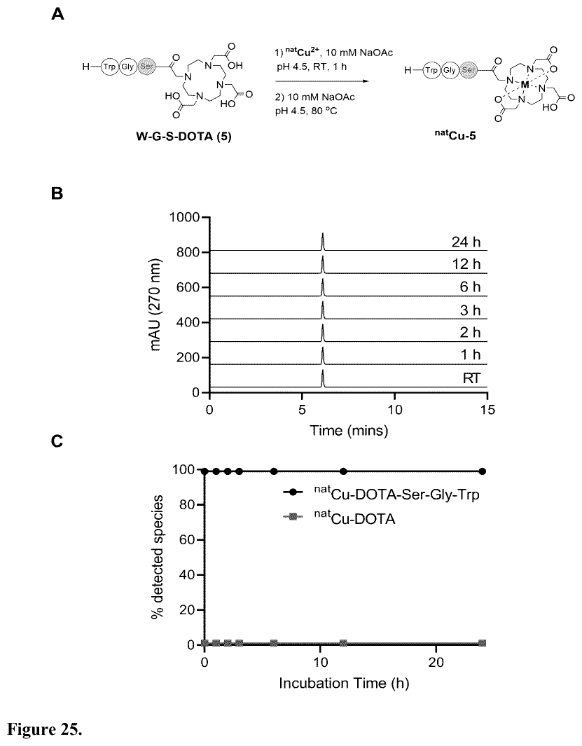

FIG. 26. (A) Schematic description of preparation and direct complexation procedure of ^{nat}Sc-5. (B) Analytical HPLC chromatograms showing no autolytic release of the ^{nat}Sc-DOTA. (C) Quantification of the pH-dependent cleavage at 80° C. and pH 4.5.

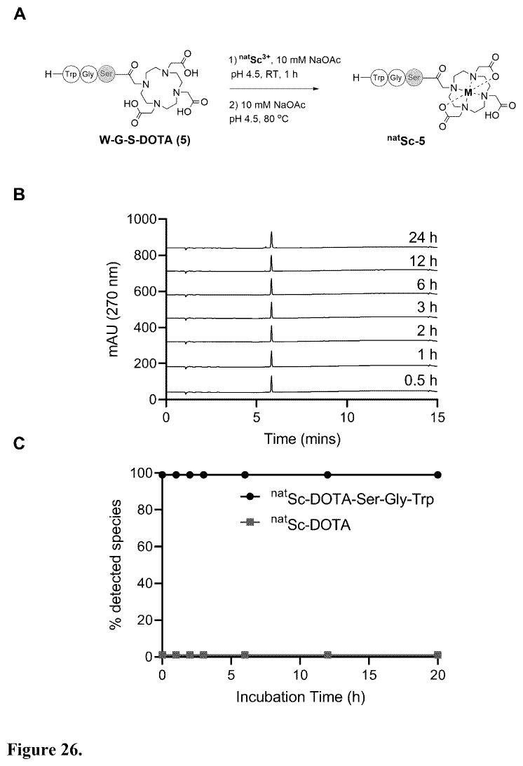

FIG. 27 Reaction scheme and corresponding radio-HPLC trace. Yields reported as an average of n=3 reactions.

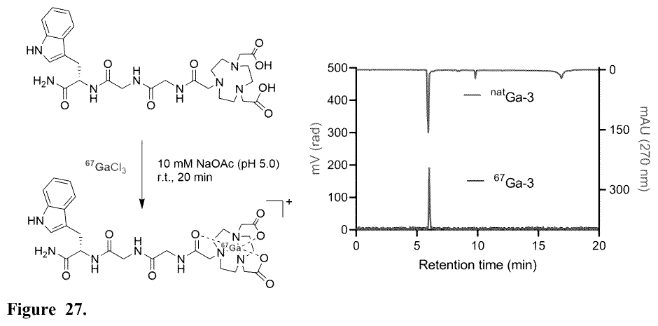

FIG. 28. Reaction scheme and corresponding radio-HPLC trace. Yields reported as an average of n=3 reactions.

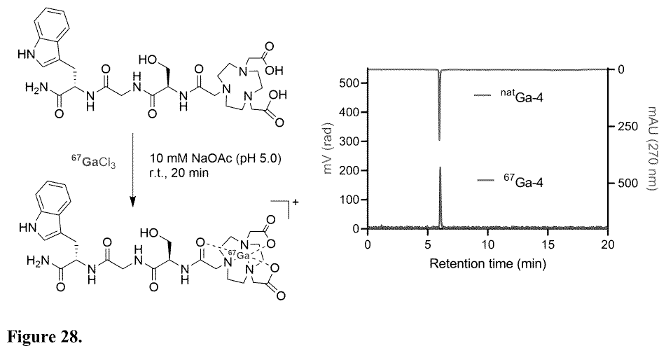

FIG. 29. Reaction scheme and corresponding radio-HPLC trace. Yields reported as an average of n=3 reactions.

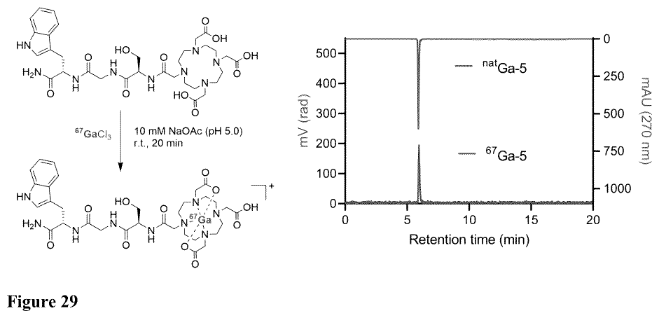

FIG. 30. Reaction scheme and corresponding radio-HPLC trace. Yields reported as an average of n=3 reactions.

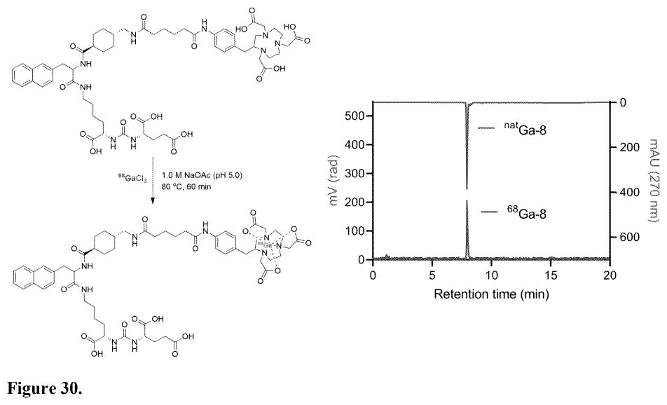

FIG. 31. (A) Schematic description of preparation and direct radiolabeling procedure of Ser-containing radiopharmaceutical. (B) Analytical radioHPLC chromatograms showing autolytic release of the desired product ⁶⁷Ga-NOTA. (C) Quantification of the temperature-dependent release of radiopharmaceutical from ⁶⁷Ga-NOTA-S-G-W at 80° C. RCY: Radiochemical Yield. Yields reported as an average of n=3 reactions.

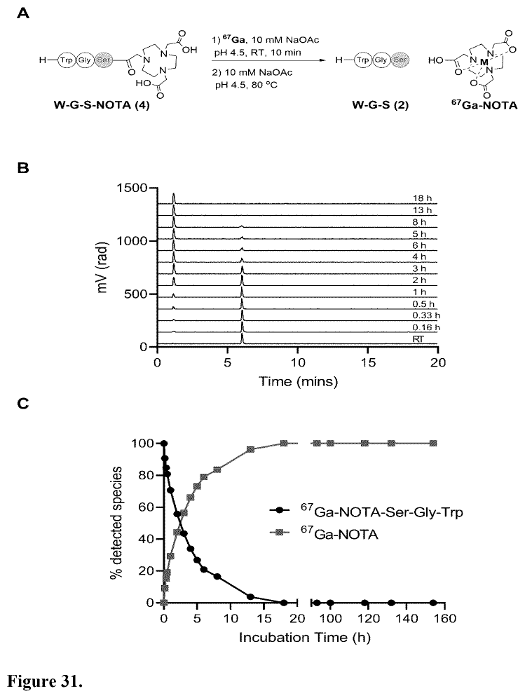

FIG. 32. (A) Schematic description of preparation and direct radiolabeling procedure of Ser-containing radiopharmaceutical. (B) Analytical radioHPLC chromatograms showing autolytic release of the desired product ⁶⁷Ga-NOTA. (C) Quantification of the temperature-dependent release of radiopharmaceutical from ⁶⁷Ga-NOTA-S-G-W at 37° C. RCY: Radiochemical Yield. Yields reported as an average of n=3 reactions.

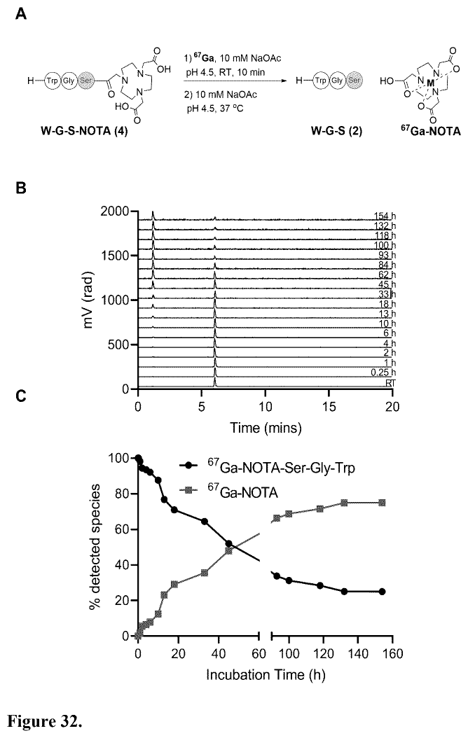

FIG. 33. (A) Schematic description of preparation and direct radiolabeling procedure of Ser-containing radiopharmaceutical. (B) Analytical radioHPLC chromatograms showing autolytic release of the desired product ⁶⁷Ga-NOTA. (C) Quantification of the temperature-dependent release of radiopharmaceutical from ⁶⁷Ga-NOTA-S-G-W at RT. RCY: Radiochemical Yield. Yields reported as an average of n=3 reactions.

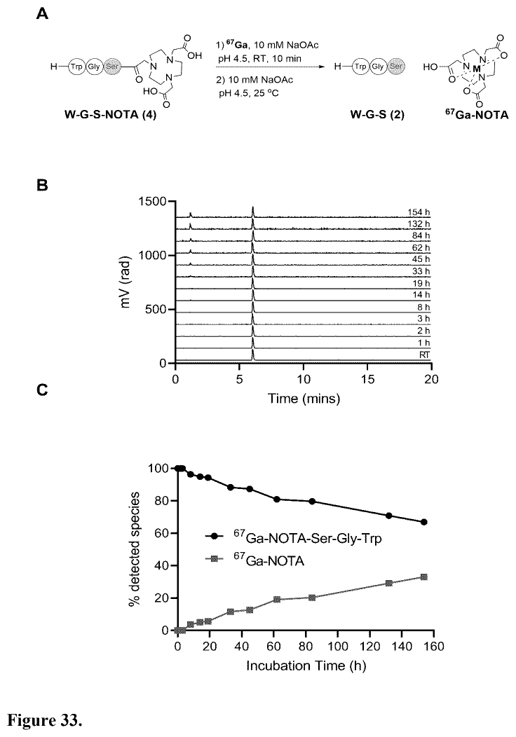

FIG. 34. (A) Schematic description of preparation and direct radiolabeling procedure of Gly-containing radiopharmaceutical. (B) Analytical radioHPLC chromatograms showing autolytic release of the desired product ⁶⁷Ga-NOTA. (C) Quantification of the temperature-dependent release of radiopharmaceutical from ⁶⁷Ga-NOTA-G-G-W at 80° C. RCY: Radiochemical Yield. Yields reported as an average of n=3 reactions.

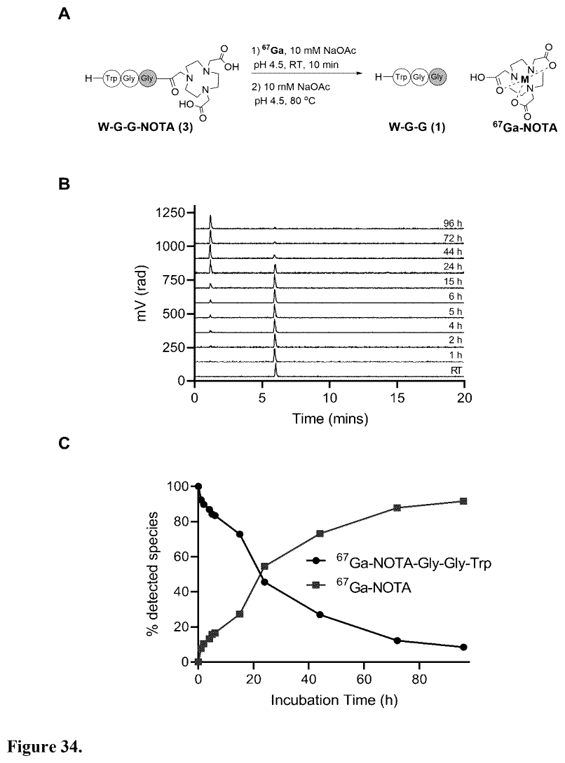

FIG. 35. (A) Schematic description of preparation and direct radiolabeling procedure of Gly-containing radiopharmaceutical. (B) Analytical radioHPLC chromatograms showing lack of fast autolytic release. (C) Quantification of the temperature-dependent release of radiopharmaceutical from ⁶⁷Ga-NOTA-G-G-W at 37° C. RCY: Radiochemical Yield. Yields reported as an average of n=3 reactions.

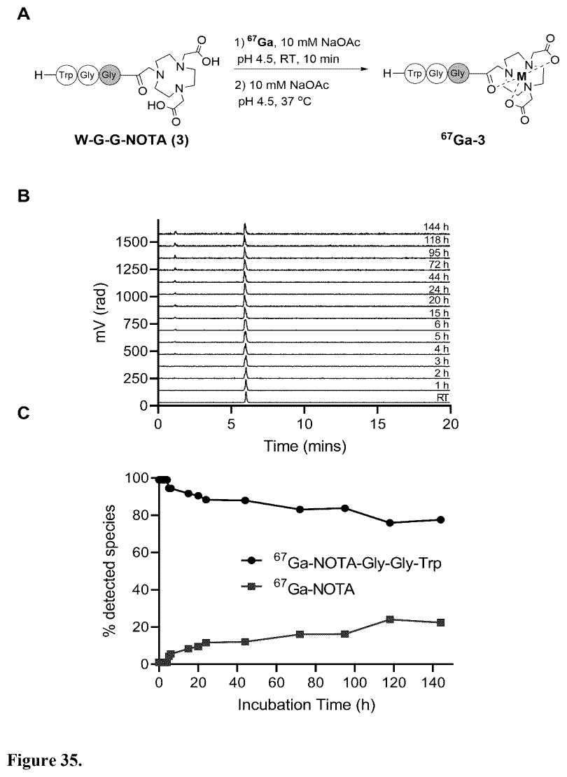

FIG. 36. (A) Schematic description of preparation and direct radiolabeling procedure of Gly-containing radiopharmaceutical. (B) Analytical radioHPLC chromatograms showing no autolytic release. (C) Quantification of the temperature-dependent release of radiopharmaceutical from ⁶⁷Ga-NOTA-G-G-W at 25° C. RCY: Radiochemical Yield. Yields reported as an average of n=3 reactions.

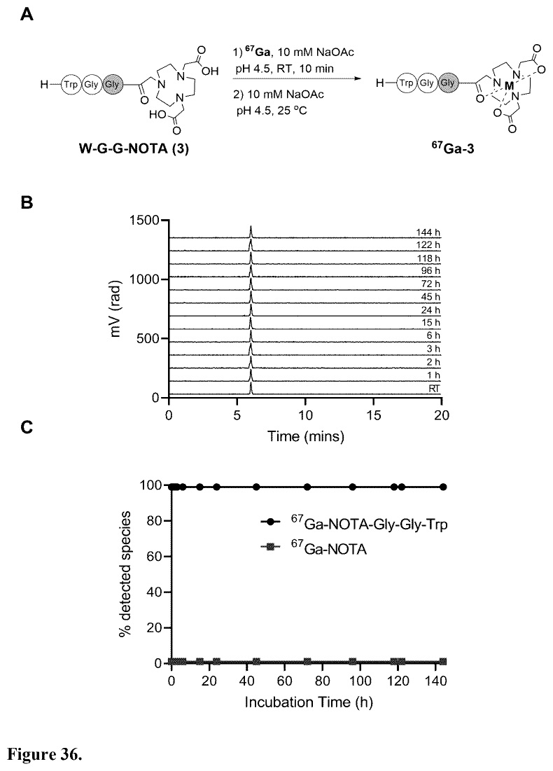

FIG. 37. (A) Schematic description of preparation and direct radiolabeling procedure of Ser-containing radiopharmaceutical. (B) Analytical radioHPLC chromatograms showing no autolytic release. (C) Quantification of the temperature-dependent release of radiopharmaceutical from ⁶⁷Ga-DOTA-S-G-W at 80° C. RCY: Radiochemical Yield. Yields reported as an average of n=3 reactions.

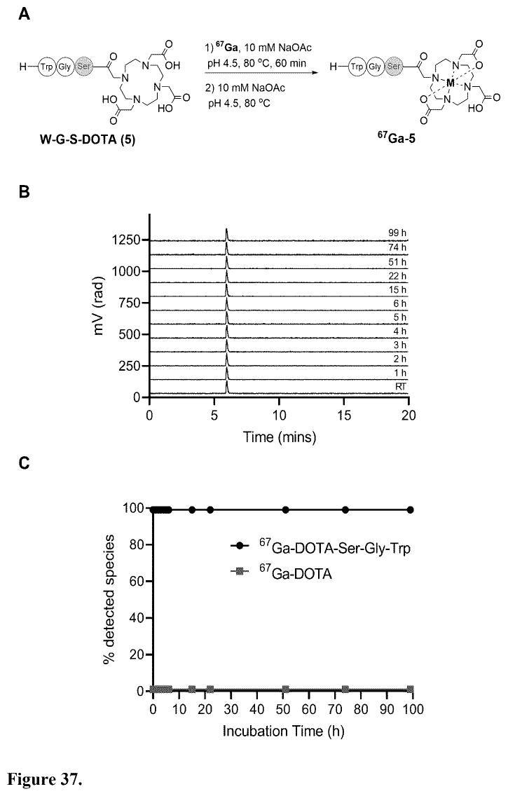

FIG. 38. (A) Schematic description of preparation and direct radiolabeling procedure of Ser-containing radiopharmaceutical. (B) Analytical radioHPLC chromatograms showing no autolytic release. (C) Quantification of the temperature-dependent release of radiopharmaceutical from ⁶⁷Ga-DOTA-S-G-W at 37° C. RCY: Radiochemical Yield. Yields reported as an average of n=3 reactions.

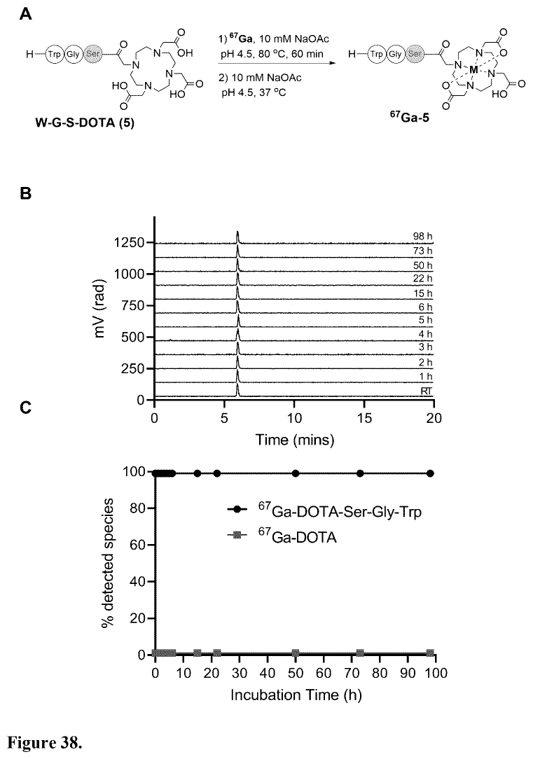

FIG. 39. (A) Schematic description of preparation and direct radiolabeling procedure of Ser-containing radiopharmaceutical. (B) Analytical radioHPLC chromatograms showing no autolytic release. (C) Quantification of the temperature-dependent release of radiopharmaceutical from ⁶⁷Ga-DOTA-S-G-W at 25° C. RCY: Radiochemical Yield. Yields reported as an average of n=3 reactions.

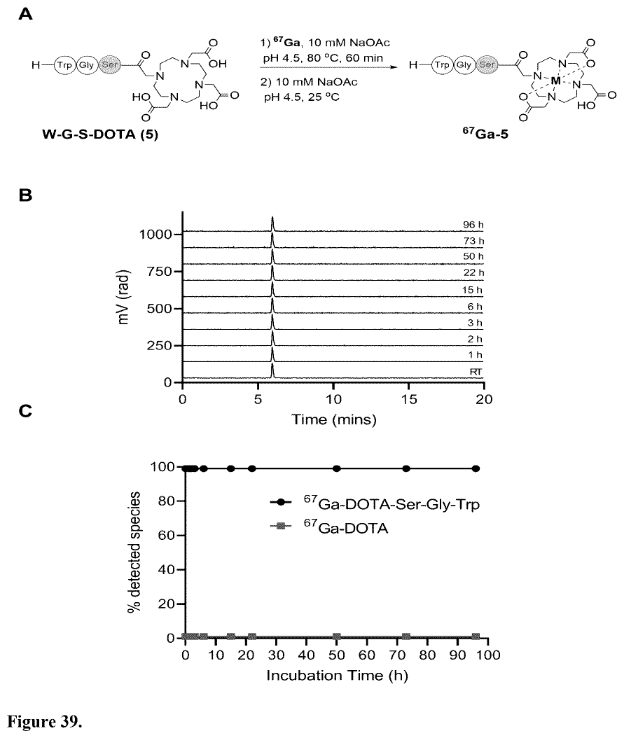

FIG. 40. Variable Temperature mass spectra of ^{a}Ga-4 complex plotted in terms of differences in mass between the unhydrated complex and each hydrated complex.

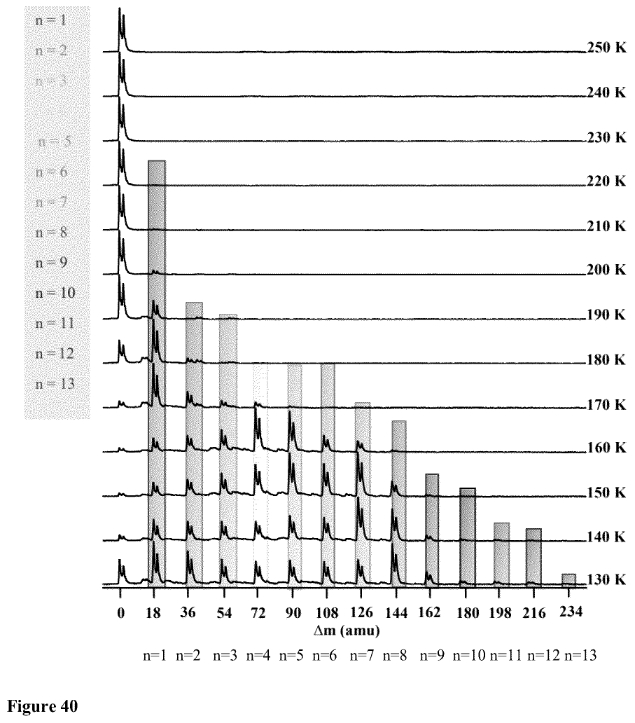

FIG. 41. Derived speciation curves of the ^{nat}Ga-4 complex showing the absence of inner sphere water. Complexes containing the same number of water adducts are depicted by colored boxes that match the speciation plot assignments.

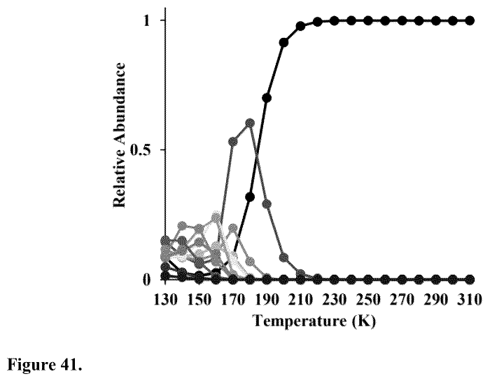

FIG. 42. Variable Temperature mass spectra of Tripeptide+NOTA plotted in terms of differences in mass between the unhydrated complex and each hydrated complex.

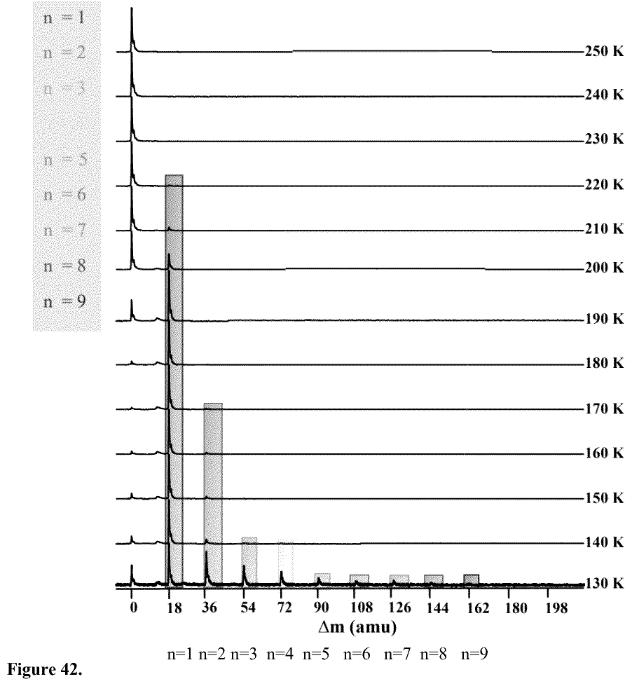

FIG. 43. Derived speciation curves of the tripeptide+NOTA depicting the incorporation of water adducts. Complexes containing the same number of water adducts are depicted by colored boxes that match the speciation plot assignments.

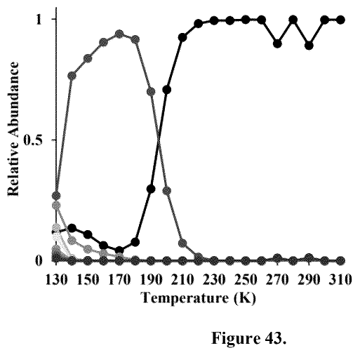

FIG. 44. CIVP spectrum of the N₂-tagged Tripeptide-NOTA (pink) and the N₂-tagged isotopically labeled Tripeptide-NOTA (green).

FIG. 45. CIVP spectrum of the N₂-tagged Ga-NOTA (^{nat}Ga-4) complex (red) and the N₂-tagged Ga-NOTA (^{nat}Ga-4*) complex with isotopically labeled serine (teal).

FIG. 46. (A) Reaction scheme for the proof-of-concept systems based on compound 10 with amide bond cleavage in solution. (B) Quantification of the temperature-dependent release of radiopharmaceutical from ⁶⁷Ga-10 at 37° C. (C) Quantification of the temperature-dependent release of radiopharmaceutical from ⁶⁷Ga-10 at 80° C. Yields reported as an average of n=3 reactions.

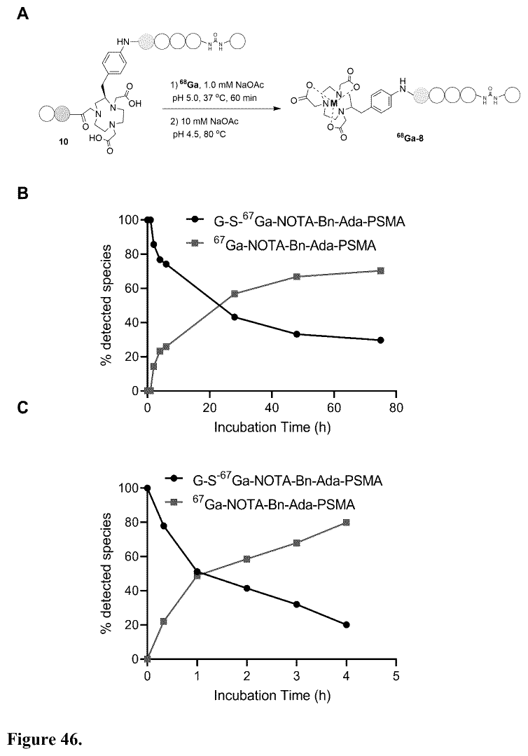

FIG. 47 A. Reagents and conditions for NOTA-ACA-BBN deprotection. FIG. 47 B. Radiolabeling reaction and data.

### DETAILED DESCRIPTION OF THE INVENTION

The present invention provides a process for preparing a compound of formula I or a compound of formula II, or a pharmaceutically acceptable salt of the compound of formula I or the compound of formula II:

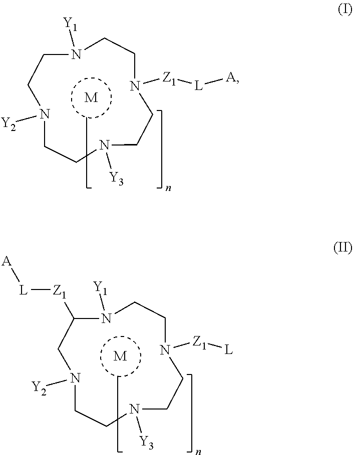

wherein
A is a targeting moiety, or an activating group or absent;
L is a peptide linker;
M is a metal that is either absent or present;
n is 0 or 1;
Y₁, Y₂, Y₃ are each, independently, —H, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H), alkyl-N(alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ and alkylheteroaryl-P(O)(OH)₂, and
wherein each occurrence of R₄ is independently, —H, alkyl, alkenyl, alkynyl, alkyl-aryl, alkyl-heteroaryl, aryl, heteroaryl, alkyl-CF₃ or —Si(alkyl)₃;
Z₁ is

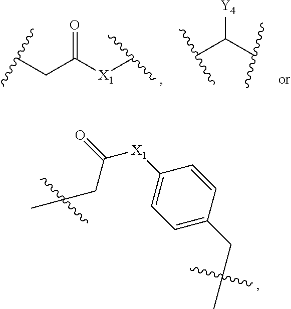

wherein X₁ is NH, O or S, and
Y₄ is —CO₂H, —CO₂R₅ aryl-CO₂H, heteroaryl-CO₂H, aryl-CO₂R₅ or heteroaryl-CO₂R₅, and wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
   wherein if the process is for producing compound of formula I when M is not present, then the process comprises:
(a) preparing a compound of formula I-A,

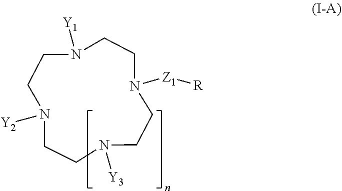

and
(b) cleaving the compound of formula I-A or the compound of II-A off a solid support surface;
   wherein if the process is for producing compound of formula I when M is present, then the process comprises:
(a) preparing a compound of formula I-A,

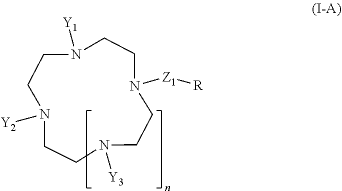

(b) complexing a metal ion with the compound of formula I-A, and
(c) cleaving the compound of formula I-A off a solid support surface;
   wherein if the process is for producing compound of formula II when M is not present, then the process comprises:
(a) preparing a compound of formula II-A,

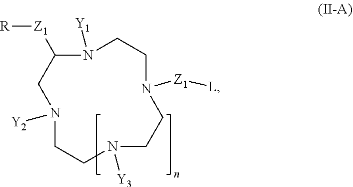

and
(b) cleaving the compound of formula II-A off a solid support surface;
   wherein if the process is for producing compound of formula I when M is present, then the process comprises:
(a) preparing a compound of formula II-A,

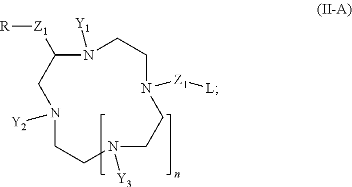

(b) complexing a metal ion with the compound of formula I-A, and
(c) cleaving the compound of formula II-A off a solid support surface;
   wherein R is

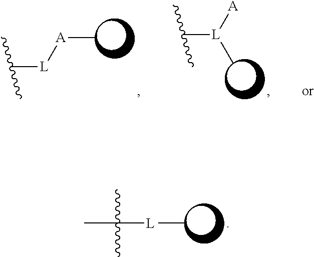

The present invention provides the following steps for producing the compound of formula I-A:
a) coupling a compound containing groups A and L to a solid support surface;
b) coupling the compound in step (a) with a compound of formula (III) using a coupling reagent in presence of base and a solvent:

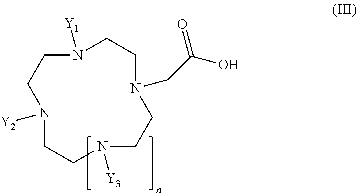

wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃;
c) reacting the compound obtained in step (b) with the following compound using a coupling reagent in presence of base and a solvent:

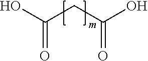

where in m is 1-20; and
d) adding an active ester synthesis reagent.

In some embodiments of preparing compound of formula I-A, the compound in step (a) contains a Fmoc protecting group and a terminal NH₂.

In some embodiments of preparing compound of formula I-A, the base used in steps b) and c) are the same base.

In some embodiments of preparing compound of formula I-A, the base is a non-nucleophilic base.

In some embodiments of preparing compound of formula I-A, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases.

In some embodiments of preparing compound of formula I-A, the base is DIEA.

In some embodiments of preparing compound of formula I-A, the coupling reagent is dicyclohexylcarbodiimide (DCC), ethyl(dimethylaminopropyl) carbodiimide (EDC), benzotriazole-1-yl-oxy-tris-pyrrolidino-phosphonium hexafluorophosphate (PyBOP), benzotriazol-1-yloxytris(dimethylamino)phosphoniumhexafluorophosphate (BOP), or 2-(1H-Benzotriazole-1-yl)-1,1,3,3-tetramethyluronium hexafluorophosphate (HBTU).

In some embodiments of preparing compound of formula I-A, the solvent is dihydrolevoglucosenone, γ-valerolactone, or dimethylformamide (DMF).

In some embodiments of preparing compound of formula I-A, the active ester synthesis reagent is N, N′-disuccinimidyl carbonate or N-Hydroxysuccinimide (NHS).

In some embodiments of preparing compound of formula I-A, the active ester synthesis reagent is NHS.

In some embodiments of preparing compound of formula I-A, m is 1-15 in step (c).

In some embodiments of preparing compound of formula I-A, m is 1-10.

In some embodiments of preparing compound of formula I-A, m is 1-5.

In some embodiments of preparing compound of formula I-A, m is 5.

In some embodiments of preparing compound of formula I-A, the solid support surface is a resin.

In some embodiments of preparing compound of formula I-A, the resin is a rink amide resin, a wang resin, an agarose resin, a tentagel resin or an ion-exchange resin.

In some embodiments of preparing compound of formula I-A, the resin is a tentagel resin.

In some embodiments of preparing compound of formula I-A, steps (a)-(d) are conducted in one pot.

In some embodiments of producing the compound of formula I when M is not present, step (a) and step (b) are conducted in one pot.

In some embodiments of producing the compound of formula I when M is not present, the compound of formula I-A is photo cleaved at a wavelength about 300-400 nm.

In some embodiments of producing the compound of formula I when M is not present, the compound of formula I-A is photo cleaved at a wavelength about 350-380 nm.

In some embodiments of producing the compound of formula I when M is not present, the compound of formula I-A is photo cleaved at a wavelength of 365 nm.

In some embodiments of producing the compound of formula I when M is not present, the compound of formula I-A is chemically cleaved.

In some embodiments of producing the compound of formula I when M is not present, the compound of formula I-A is chemically cleaved by a mixture of trifluoroacetic acid (TFA), triisopropyl silane (TIS) and water.

In some embodiments of producing the compound of formula I when M is present, step (a), step (b) and step (c) are conducted in one pot.

In some embodiments of producing the compound of formula I when M is present, the compound of formula I-A is photo cleaved at a wavelength about 300-400 nm.

In some embodiments of producing the compound of formula I when M is present, the compound of formula I-A is photo cleaved at a wavelength about 350-380 nm.

In some embodiments of producing the compound of formula I when M is present, the compound of formula I-A is photo cleaved at a wavelength of 365 nm.

In some embodiments of producing the compound of formula I when M is present, the compound of formula I-A is chemically cleaved.

In some embodiments of producing the compound of formula I when M is present, the compound of formula I-A is chemically cleaved by a mixture of trifluoroacetic acid (TFA), triisopropyl silane (TIS) and water.

In some embodiments of producing the compound of formula II when M is not present, wherein step (a) and step (b) are conducted in one pot.

In some embodiments of producing the compound of formula II when M is not present, the compound of formula II-A is photo cleaved at a wavelength about 300-400 nm.

In some embodiments of producing the compound of formula II when M is not present, the compound of formula II-A is photo cleaved at a wavelength about 350-380 nm.

In some embodiments of producing the compound of formula II when M is not present, the compound of formula II-A is photo cleaved at a wavelength of 365 nm.

In some embodiments of producing the compound of formula II when M is not present, the compound of formula II-A is chemically cleaved.

In some embodiments of producing the compound of formula II when M is not present, the compound of formula II-A is chemically cleaved by a mixture of trifluoroacetic acid (TFA), triisopropyl silane (TIS) and water; or

In some embodiments of producing the compound of formula II when M is present, step (a), step (b) and step (c) are conducted in one pot.

In some embodiments of producing the compound of formula II when M is present, the compound of formula II-A is photo cleaved at a wavelength about 300-400 nm.

In some embodiments of producing the compound of formula II when M is present, the compound of formula II-A is photo cleaved at a wavelength about 350-380 nm.

In some embodiments of producing the compound of formula II when M is present, the compound of formula II-A is photo cleaved at a wavelength of 365 nm.

In some embodiments of producing the compound of formula II when M is present, the compound of formula I-A is chemically cleaved.

In some embodiments of producing the compound of formula II when M is present, the compound of formula I-A is chemically cleaved by a mixture of trifluoroacetic acid (TFA), triisopropyl silane (TIS) and water.

In some embodiments of preparing compound of formula I-A, the compound in step (a) has the structure:

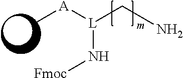

wherein m is 1-15.

In some embodiments, m is 1-10. In some embodiments, m is 1-5. In some embodiments, m is 4.

In some embodiments of preparing compound of formula I-A, the compound in step (b) has the structure:

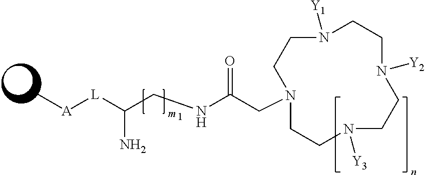

wherein Y₁, Y₂, Y₃ are each independently, alkyl-CO₂R₄ wherein R₄ is H alkyl, alkenyl, alkynyl, alkyl-aryl, alkyl-heteroaryl, aryl, heteroaryl, alkyl-CF₃.

In some embodiments, m₁ is 1-15. In some embodiments, m₁ is 1-10. In some embodiments, m₁ is 1-5. In some embodiments, m₁ is 4.

In some embodiments of preparing compound of formula I-A the compound in step (c) has the structure:

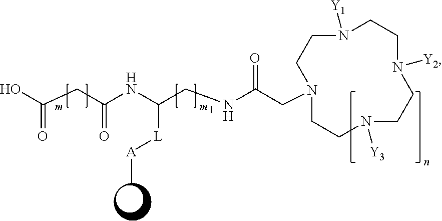

wherein m₁ is 1-15.

In some embodiments, m is 1-10. In some embodiments, m is 1-5. In some embodiments, m is 4.

In some embodiments of preparing compound of formula I-A, the compound in step (d) has the structure

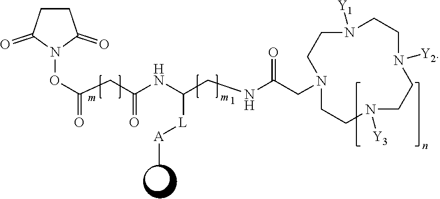

The present invention provides the following steps for producing the compound of formula I-A
a) coupling an acid containing a fluorenylmethoxycarbonyl protecting group (Fmoc) using a coupling reagent in the presence of a base to a solid support surface in a solvent;
b) deprotecting the Fmoc group with a deprotection reagent in the presence of a solvent and coupling groups A and L to the acid using a coupling reagent in the presence of a base;
c) coupling an acid containing a Fmoc protection group using a coupling reagent in the presence of a base and a solvent to the compound obtained in step (b); and
d) deprotecting the Fmoc group with a deprotection reagent and coupling the compound obtained in step (c) with the compound of formula (III) in a solvent:

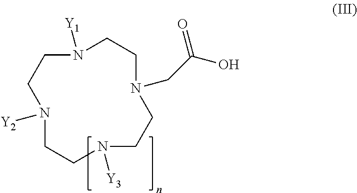

wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃.

In some embodiments of preparing compound of formula I-A, the base used in steps a), b) and c) is the same base.

In some embodiments of preparing compound of formula I-A, the base is a non-nucleophilic base.

In some embodiments of preparing compound of formula I-A, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases.

In some embodiments of preparing compound of formula I-A, the base is DIEA.

In some embodiments of preparing compound of formula I-A, the deprotection reagent is a primary, secondary or tertiary amine.

In some embodiments of preparing compound of formula I-A, the deprotection reagent is a primary or secondary amine.

In some embodiments of preparing compound of formula I-A, the deprotection reagent is a primary amine.

In some embodiments of preparing compound of formula I-A, the acid containing a Fmoc protecting group in step (a) is a carboxylic acid.

In some embodiments, the carboxylic acid has an amine group.

In some embodiments, the carboxylic acid has a Fmoc protecting group protecting the amine group.

In some embodiments of preparing compound of formula I-A, the acid containing a Fmoc protecting group is 3-(Fmoc-amino)-3-(2-nitrophenyl) propionic acid, Fmoc-Asp-OH, Fmoc-Asn-OH, 3-(Fmoc-amino)-3-(2-nitrophenyl) propionic acid, Fmoc-8-Aoc-OH, Fmoc-5-Ava-Oh, Fmoc-p-Ala-OH, Fmoc-GABA-OH, Fmoc-11-Aun-OH, Fmoc-NH-(PEG)-COOH or Fmoc-6-Ahx-OH.

In some embodiments of preparing compound of formula I-A, the acid is 3-(Fmoc-amino)-3-(2-nitrophenyl) propionic acid.

In some embodiments of preparing compound of formula I-A, the coupling reagent is dicyclohexylcarbodiimide (DCC), ethyl(dimethylaminopropyl) carbodiimide (EDC), benzotriazole-1-yl-oxy-tris-pyrrolidino-phosphonium hexafluorophosphate (PyBOP), benzotriazol-1-yloxytris(dimethylamino)phosphoniumhexafluorophosphate (BOP), or 2-(1H-Benzotriazole-1-yl)-1,1,3,3-tetramethyluronium hexafluorophosphate (HBTU).

In some embodiments of preparing compound of formula I-A, R₄ is alkyl, alkenyl, alkynyl, alkyl-CF₃ in step (d).

In some embodiments of preparing compound of formula I-A, R₁ is alkyl, alkenyl, alkynyl.

In some embodiments of preparing compound of formula I-A, R₄ is alkyl, alkenyl.

In some embodiments of preparing compound of formula I-A, R₄ is alkyl.

In some embodiments of preparing compound of formula I-A, the solvent is dihydrolevoglucosenone, γ-valerolactone, or dimethylformamide (DMF).

In some embodiments of preparing compound of formula I-A, the solid support surface is a resin.

In some embodiments of preparing compound of formula I-A, the resin is a rink amide resin, a wang resin, an agarose resin, a tentagel resin or an ion-exchange resin.

In some embodiments of preparing compound of formula I-A, the resin is a tentagel resin.

In some embodiments of preparing compound of formula I-A, in step (b), A and L are coupled sequentially.

In some embodiments of preparing compound of formula I-A, in step (b), A and L are coupled together.

In some embodiments of preparing compound of formula I-A, steps (a)-(d) are conducted in one pot.

In some embodiments of preparing compound of formula I-A, the deprotection reagent is cyclohexylamine, ethanolamine, piperidine, piperazine.

In some embodiments of preparing compound of formula I-A, the deprotection reagent is piperidine.

In some embodiments of preparing compound of formula I-A, the concentration of piperidine is about 10-50%.

In some embodiments of preparing compound of formula I-A, the concentration of piperidine is about 10-40%.

In some embodiments of preparing compound of formula I-A, the concentration of piperidine is about 10-30%.

In some embodiments of preparing compound of formula I-A, the concentration of piperidine is about 20%.

In some embodiments of preparing compound of formula I-A, R₄ is C₁_₈ alkyl.

In some embodiments of preparing compound of formula I-A, R₄ is C₁₋₆ alkyl.

In some embodiments of preparing compound of formula I-A, R₄ is C₂₋₅ alkyl.

In some embodiments of preparing compound of formula I-A, R₄ is butyl.

In some embodiments of preparing compound of formula I-A, R₄ is tert-butyl.

In some embodiments of preparing compound of formula I-A, the compound produced in step (b) has the following structure:

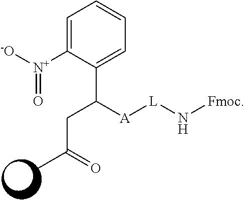

In some embodiments of preparing compound of formula I-A, the acid containing a Fmoc in step (c) has the following structure:

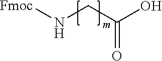

where in m is 1-20.

In some embodiments m is 1-10. In some embodiments m is 1-5. In some embodiments m is 5.

In some embodiments of preparing compound of formula I-A, the compound produced in step (c) has the following structure:

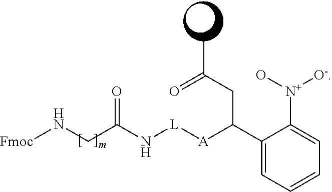

In some embodiments of preparing compound of formula I-A, the acid containing a Fmoc protecting group in step (a) is Fmoc-Trp(Boc)-OH, Fmoc-Asp-OH, Fmoc-Asn-OH, 3-(Fmoc-amino)-3-(2-nitrophenyl) propionic acid, Fmoc-8-Aoc-OH, Fmoc-5-Ava-Oh, Fmoc-p-Ala-OH, Fmoc-GABA-OH, Fmoc-11-Aun-OH, Fmoc-NH-(PEG)-COOH or Fmoc-6-Ahx-OH.

In some embodiments of preparing compound of formula I-A, the acid is Fmoc-Trp(Boc)-OH.

The present invention provides the following steps for producing the compound of formula II-A:
a) coupling an active ester synthesis reagent to the following compound in the presence of a coupling reagent and a base

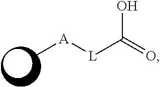

and
b) coupling the compound obtained in step (a) with the following compound in a base and a solvent

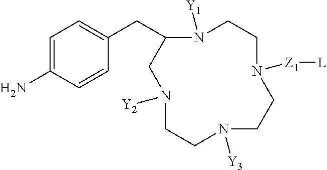

wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃.

In some embodiments of preparing compound of formula II-A, the base used in steps a) and b) is the same base.

In some embodiments of preparing compound of formula II-A, the base is a non-nucleophilic base.

In some embodiments of preparing compound of formula II-A, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases.

In some embodiments of preparing compound of formula II-A, the base is DIEA.

In some embodiments of preparing compound of formula II-A, the active ester synthesis reagent is N, N′-disuccinimidyl carbonate or N-Hydroxysuccinimide (NHS).

In some embodiments of preparing compound of formula II-A, the active ester synthesis reagent NHS.

In some embodiments of preparing compound of formula II-A, the coupling reagent is dicyclohexylcarbodiimide (DCC), ethyl(dimethylaminopropyl) carbodiimide (EDC), benzotriazole-1-yl-oxy-tris-pyrrolidino-phosphonium hexafluorophosphate (PyBOP), benzotriazol-1-yloxytris(dimethylamino)phosphoniumhexafluorophosphate (BOP), or 2-(1H-Benzotriazole-1-yl)-1,1,3,3-tetramethyluronium hexafluorophosphate (HBTU); preferably EDC.

In some embodiments of preparing compound of formula II-A, the solid support surface is a resin.

In some embodiments of preparing compound of formula II-A, the resin is a rink amide resin, a wang resin, an agarose resin, a tentagel resin or an ion-exchange resin.

In some embodiments of preparing compound of formula II-A, the resin is a wang resin.

In some embodiments of preparing compound of formula II-A, the solvent in step (b) is dihydrolevoglucosenone, γ-valerolactone or dimethylformamide (DMF).

In some embodiments of preparing compound of formula II-A, step (b) is performed at a temperate of 30-80° C.

In some embodiments of preparing compound of formula II-A, step (b) is performed at a temperate of 40-60° C.

In some embodiments of preparing compound of formula II-A, step (b) is performed at a temperate of 50° C.

In some embodiments of preparing compound of formula II-A, steps (a)-(b) are conducted in one pot.

In some embodiments of preparing a compound of formula I or a compound of formula II, R₄ is C₁-C₈ alkyl.

In some embodiments of preparing a compound of formula I or a compound of formula II, R₄ is C₁-C₆ alkyl.

In some embodiments of preparing a compound of formula I or a compound of formula II, R₄ is C₃-C₆ alkyl.

In some embodiments of preparing a compound of formula I or a compound of formula II, R₄ is butyl.

In some embodiments of preparing a compound of formula I or a compound of formula II, R₄ is tert-butyl.

In some embodiments of preparing a compound of formula I or a compound of formula II, L contains at least two, three, four, five or six amino acids.

In some embodiments of preparing a compound of formula I or a compound of formula II, the peptide linker contains at least three, four, five amino acids.

In some embodiments of preparing a compound of formula I or a compound of formula II, the peptide linker contains at least three or four amino acids.

In some embodiments of preparing a compound of formula I or a compound of formula II, the peptide linker contains three amino acids.

In some embodiments of preparing a compound of formula I or a compound of formula II, L contains 6-aminohexanoic acid (Aca), 3-amino-3-(2-nitrophenyl)propionic acid (Anp) and pyroglutamic acid.

In some embodiments of preparing a compound of formula I or a compound of formula II, A is a targeting moiety.

In some embodiments of preparing a compound of formula I or a compound of formula II, the targeting moiety is trastuzumab, bombesin, somatostatin or 2-[3-(1,3-dicarboxypropyl)ureido]pentanedioic acid (DUPA) or a derivative or fragment thereof.

In some embodiments of preparing a compound of formula I or a compound of formula II, A is activating group.

In some embodiments of preparing a compound of formula I or a compound of formula II, the activating group is to activate carboxylic acids.

In some embodiments of preparing a compound of formula I or a compound of formula II, the activating group is Bis(pentafluorophenyl) carbonate or N-Hydroxysuccinimide.

In some embodiments of preparing a compound of formula I or a compound of formula II, R₁, R₂, R₃ are independently OH or t-butyl.

In some embodiments of preparing a compound of formula I or a compound of formula II, R₁, R₂, R₃ are OH.

In some embodiments of preparing a compound of formula I or a compound of formula II, R₁, R₂, R₃ are t-butyl.

In some embodiments of preparing a compound of formula I or a compound of formula II, L contains amino acids selected from serine (ser/S), glycine (gly/G), tryptophan (trp/W), glutamine (gln/Q), arginine (arg/R), leucine (leu/L), asparagine (asn/N), alanine (ala/A), valine (val/V), histidine (his/H), and methionine (met/M).

In some embodiments of preparing a compound of formula I or a compound of formula II, L contains amino acids selected from Q, W and G.

In some embodiments of preparing a compound of formula I or a compound of formula II, L contains amino acids selected from Q and G.

In some embodiments of preparing a compound of formula I or a compound of formula II, L contains Aca-gln-trp.

In some embodiments of preparing a compound of formula I or a compound of formula II, L contains gln-trp-ala-val-gly-his-leu-met.

In some embodiments of preparing a compound of formula I or a compound of formula II, L contains gly-gly-trp.

In some embodiments of preparing a compound of formula I or a compound of formula II, L contains Aca-bombesin (BBN).

In some embodiments of preparing a compound of formula I or a compound of formula II, L contains ser-gly-trp. In some embodiments of preparing a compound of formula I or a compound of formula II, L contains Aca-gln-trp-anp.

The present invention provides the following steps for producing the compound of formula II-A:
a) reacting a compound of formula IV

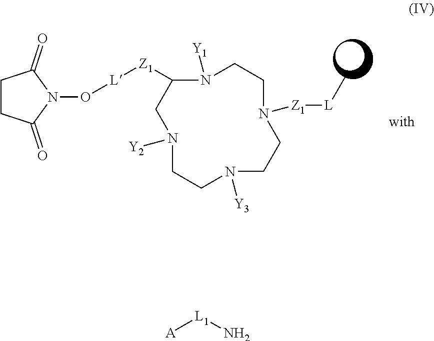

wherein L′ and L₁ are peptide linkers.

In some embodiments of preparing a compound of formula II-A, the reaction is conducted in base.

In some embodiments of preparing a compound of formula II-A, the base is a non-nucleophilic base.

In some embodiments of preparing a compound of formula II-A, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases.

In some embodiments of preparing a compound of formula II-A, the base is DIEA.

In some embodiments of preparing a compound of formula II-A, the reaction is conducted at a temperate of 30-80° C.

In some embodiments of preparing a compound of formula II-A, the reaction is conducted at a temperate of 40-60° C.

In some embodiments of preparing a compound of formula II-A, the reaction is conducted at a temperate of 50° C.

The present invention provides the following steps for producing the compound of formula IV:
a) coupling an acid containing a fluorenylmethoxycarbonyl protecting group (Fmoc) using a coupling reagent in the presence of a base to a solid support surface in a solvent;
b) deprotecting the Fmoc group with a deprotection reagent in the presence of a solvent and coupling groups L to the acid using a coupling reagent in the presence of a base;
c) coupling an acid containing a Fmoc protection group using a coupling reagent in the presence of a base and a solvent to the compound obtained in step (b); and
d) deprotecting the Fmoc group with a deprotection reagent and coupling the compound obtained in step (c) with the compound of formula IV-A in a solvent:

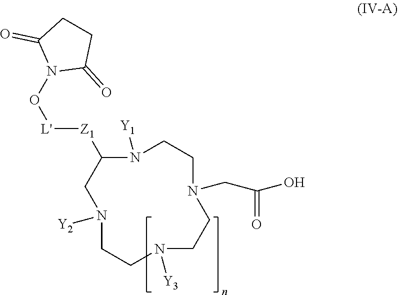

wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃.

In some embodiments, the compound of formula I has the following structure:

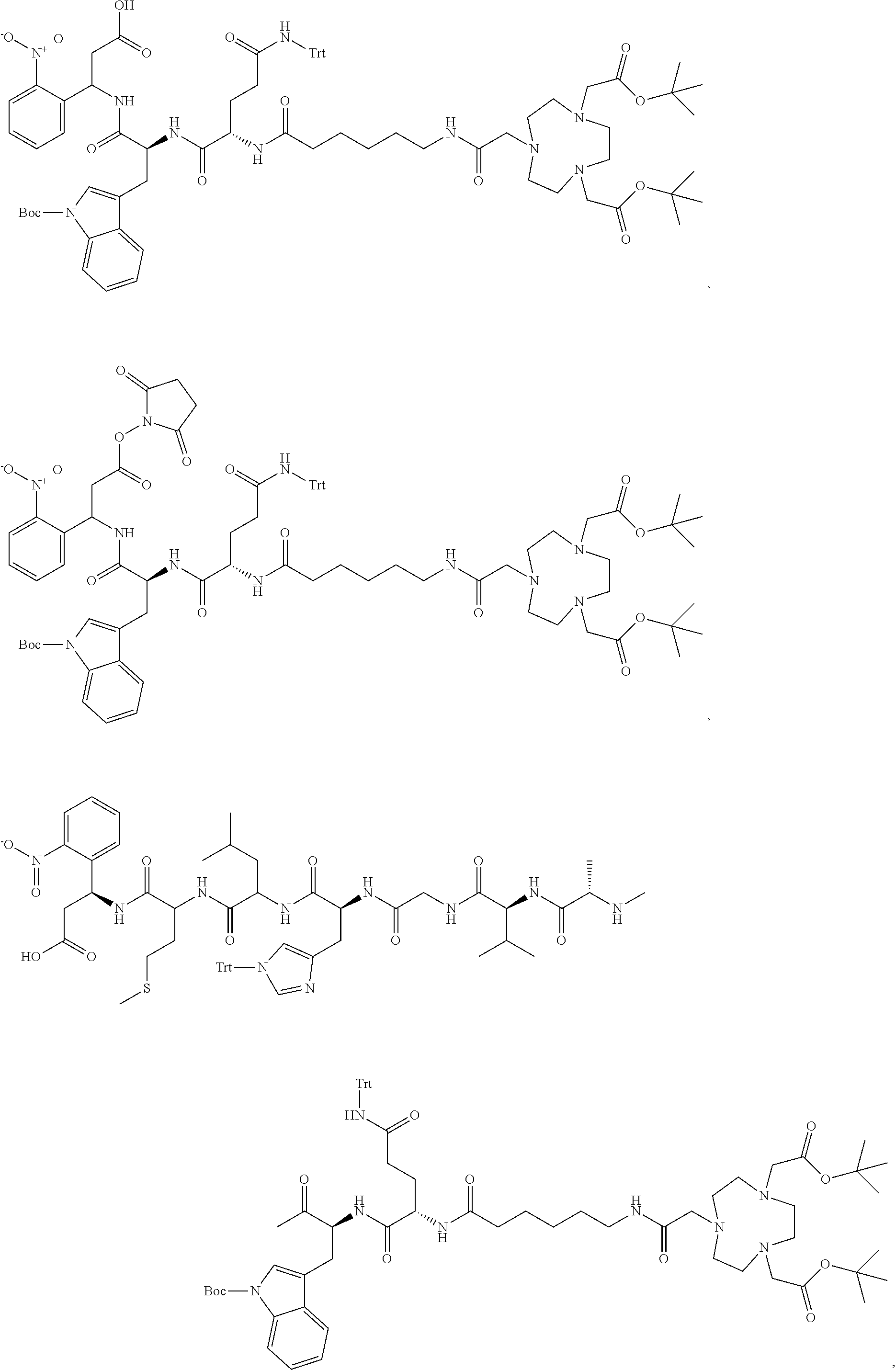

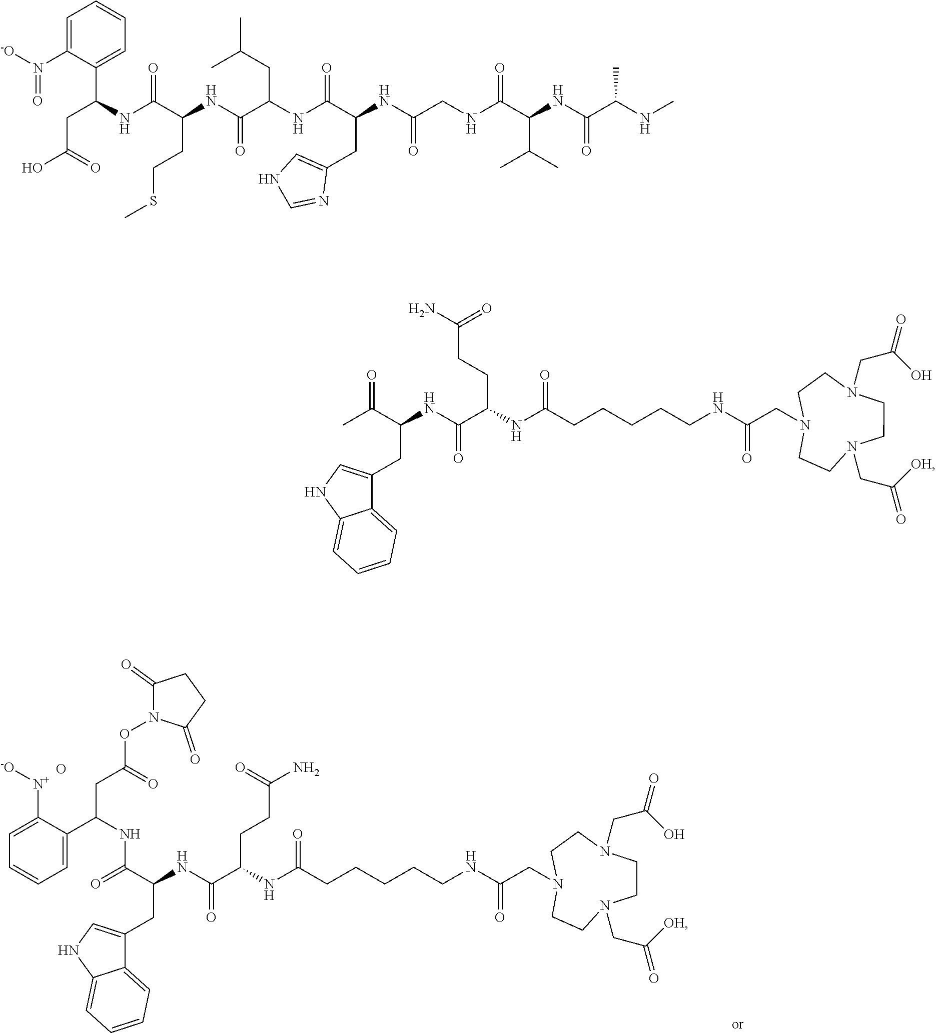

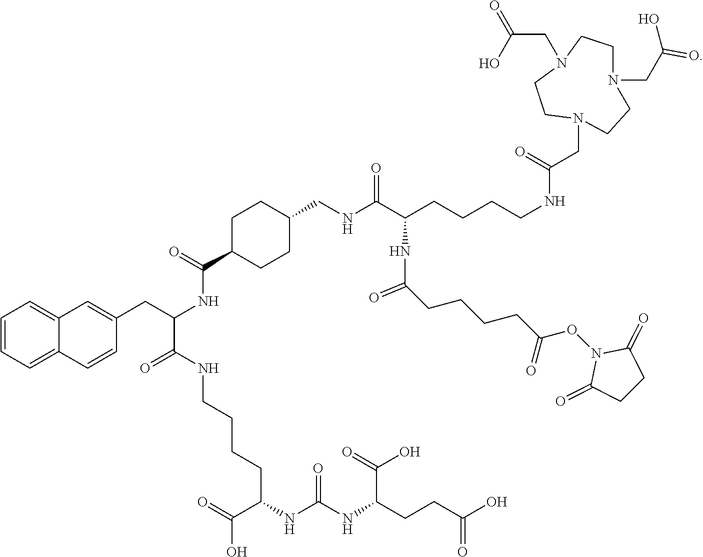 [SMILES: O=C(O)CC[C@H](NC(=O)N[C@@H](CCCCNC(=O)C(Cc1ccc2ccccc2c1)NC(=O)[C@H]1CC[C@H](CNC(=O)[C@H](CCCCNC(=O)CN2CCN(CC(=O)O)CCN(CC(=O)O)CC2)NC(=O)CCCCC(=O)ON2C(=O)CCC2=O)CC1)C(=O)O)C(=O)O]

In some embodiments, the compound of formula I has the following structure:

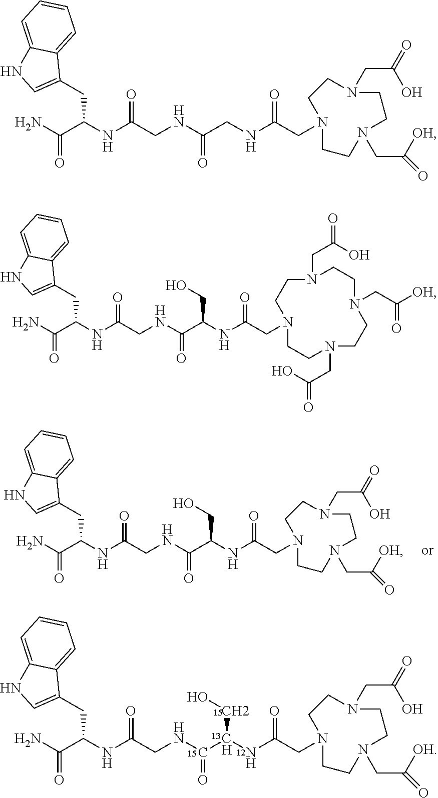 [SMILES: NC(=O)[C@H](Cc1c[nH]c2ccccc12)NC(=O)CNC(=O)CNC(=O)CN1CCN(CC(=O)O)CCN(CC(=O)O)CC1 ; NC(=O)[C@H](Cc1c[nH]c2ccccc12)NC(=O)CNC(=O)[C@@H](CO)NC(=O)CN1CCN(CC(=O)O)CCN(CC(=O)O)CCN(CC(=O)O)CC1 ; NC(=O)[C@H](Cc1c[nH]c2ccccc12)NC(=O)CNC(=O)[C@@H](CO)NC(=O)CN1CCN(CC(=O)O)CCN(CC(=O)O)CC1 ; NC(=O)[C@H](Cc1c[nH]c2ccccc12)NC(=O)CNC(=O)[C@@H](CO)NC(=O)CN1CCN(CC(=O)O)CCN(CC(=O)O)CC1]

In some embodiments, the compound of formula II has the following structure:

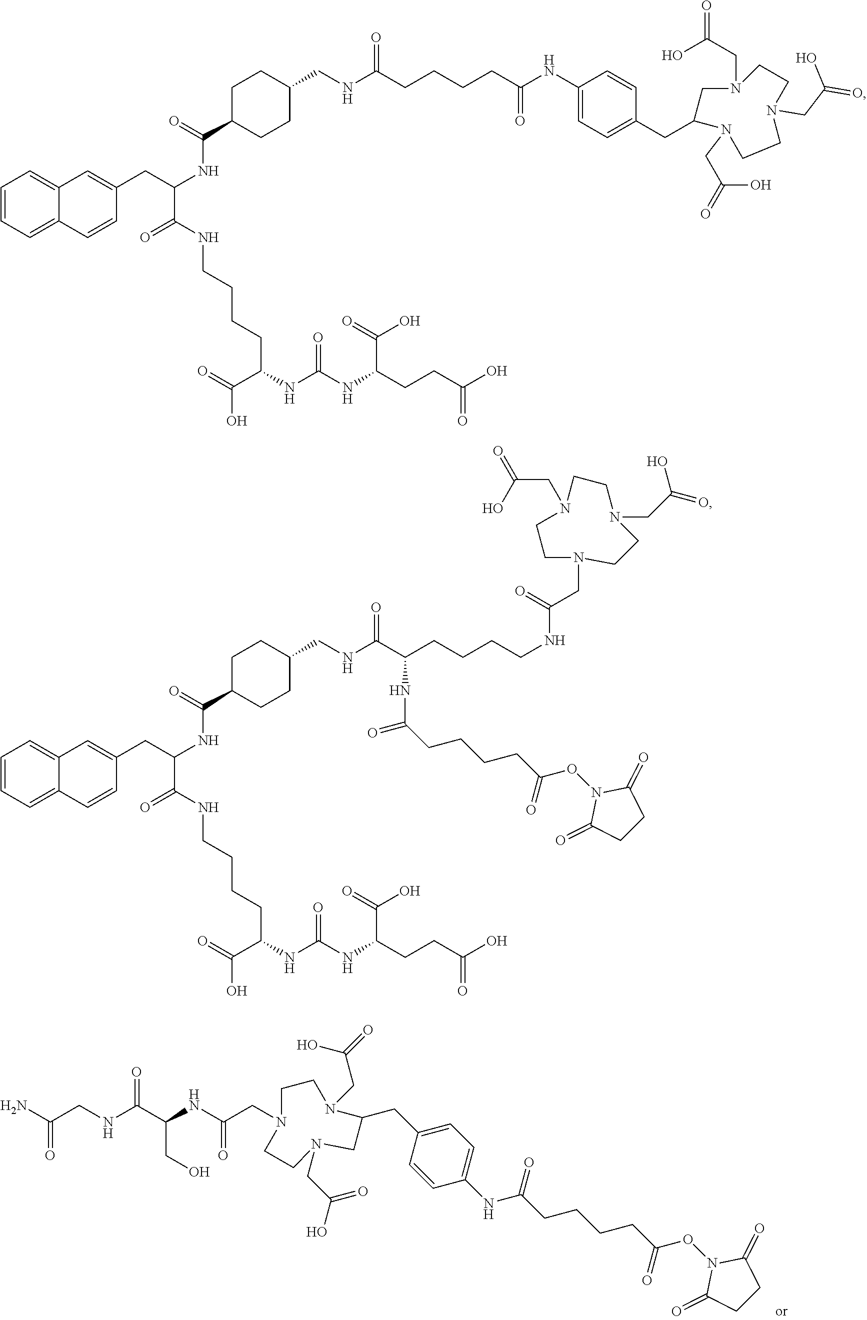

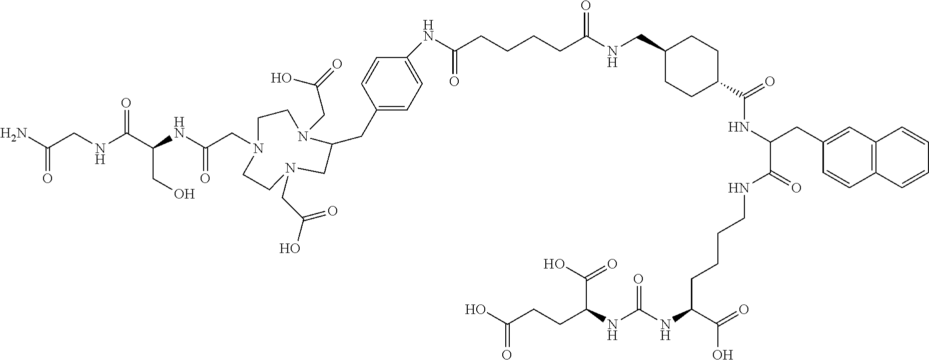 [SMILES: NC(=O)CNC(=O)[C@H](CO)NC(=O)CN1CCN(CC(=O)O)CC(Cc2ccc(NC(=O)CCCCC(=O)NC[C@H]3CC[C@H](C(=O)NC(Cc4ccc5ccccc5c4)C(=O)NCCCC[C@H](NC(=O)N[C@@H](CCC(=O)O)C(=O)O)C(=O)O)CC3)cc2)N(CC(=O)O)CC1]

In some embodiments for producing the compound of formula I when M is present, the metal ion is added in the presence of a buffer solution.

In some embodiments for producing the compound of formula I when M is present, the metal ion is a radioactive metal ion.

In some embodiments for producing the compound of formula I when M is present, the temperature of step (c) is at room temperature.

In some embodiments for producing the compound of formula I when M is present, the temperature of step (c) is around 40-90° C.

In some embodiments for producing the compound of formula I when M is present, the temperature of step (c) is around 60-90° C.

In some embodiments for producing the compound of formula I when M is present, the temperature of step (c) is around 70-90° C.

In some embodiments for producing the compound of formula I when M is present, the temperature of step (c) is 80° C.

In some embodiments for producing the compound of formula I when M is present, the pH of step (c) is 3-8.

In some embodiments for producing the compound of formula I when M is present, the pH of step (c) is 4-7.

In some embodiments for producing the compound of formula I when M is present, the pH of step (c) is 4.5-6.5.

In some embodiments for producing the compound of formula I when M is present, the buffer solution is sodium acetate (NaOAc) or N-2-hydroxyethylpiperazine-N′-2-ethanesulfonic acid (HEPES).

In some embodiments for producing the compound of formula II when M is present, the metal ion is added in the presence of a buffer solution.

In some embodiments for producing the compound of formula II when M is present, the metal ion is a radioactive metal ion.

In some embodiments for producing the compound of formula II when M is present, the temperature of step (c) is at room temperature.

In some embodiments for producing the compound of formula II when M is present, the temperature of step (c) is around 40-90° C.

In some embodiments for producing the compound of formula II when M is present, the temperature of step (c) is around 60-90° C.

In some embodiments for producing the compound of formula II when M is present, the temperature of step (c) is around 70-90° C.

In some embodiments for producing the compound of formula II when M is present, the temperature of step (c) is 80° C.

In some embodiments for producing the compound of formula II when M is present, the pH of step (c) is 3-8.

In some embodiments for producing the compound of formula II when M is present, the pH of step (c) is 4-7.

In some embodiments for producing the compound of formula II when M is present, the pH of step (c) is 4.5-6.5.

In some embodiments for producing the compound of formula II when M is present, the buffer solution is sodium acetate (NaOAc) or N-2-hydroxyethylpiperazine-N′-2-ethanesulfonic acid (HEPES).

In some embodiments for producing the compound of formula I when M is present, the buffer solution is sodium acetate (NaOAc) or N-2-hydroxyethylpiperazine-N′-2-ethanesulfonic acid (HEPES).

In some embodiments for producing the compound of formula I when M is present, the pH of the metal ion solution is around 4-6.

In some embodiments for producing the compound of formula I when M is present, the pH of the metal ion solution is 5.

In some embodiments for producing the compound of formula II when M is present, the buffer solution is sodium acetate (NaOAc) or N-2-hydroxyethylpiperazine-N′-2-ethanesulfonic acid (HEPES).

In some embodiments for producing the compound of formula II when M is present, the pH of the metal ion solution is around 4-6.

In some embodiments for producing the compound of formula II when M is present, the pH of the metal ion solution is 5.

In some embodiments for producing the compound of formula I and II, wherein the metal ion is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb); preferably, the metal is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (44Sc), Scandium-47 (⁴⁷Sc), or Scandium-43 (⁴³Sc); more preferably, the metal is Gallium-67 (Ga⁶⁷), Gallium-68 (Ga⁶⁸), Copper-62 (⁶²Cu), Copper-64 (⁶⁴Cu), or Copper-67 (⁶⁷Cu).

In some embodiments, the radiochemical yield of preparing the metal complex is at least 90%.

In some embodiments, the radiochemical yield of preparing the metal complex is at least 92%.

In some embodiments, the radiochemical yield of preparing the metal complex is at least 94%.

In some embodiments, the radiochemical yield of preparing the metal complex is at least 96%.

In some embodiments, the radiochemical yield of preparing the metal complex is at least 98%.

In some embodiments, the radiochemical yield of preparing the metal complex is at least 99%.

In some embodiments, the photocleave yield of cleaving the compound of formula I-A and formula II-A is at least 40%.

In some embodiments, the photocleave yield of cleaving the compound of formula I-A and formula II-A is at least 50%.

In some embodiments, the photocleave yield of cleaving the compound of formula I-A and formula II-A is at least 60%.

In some embodiments, the photocleave yield of cleaving the compound of formula I-A and formula II-A is at least 70%.

In some embodiments, the photocleave yield of cleaving the compound of formula I-A and formula II-A is at least 80%.

In some embodiments, the photocleave yield of cleaving the compound of formula I-A and formula II-A is at least 90%.

In some embodiments, the photocleave yield of cleaving the compound of formula I-A and formula II-A is at least 92%.

In some embodiments, the photocleave yield of cleaving the compound of formula I-A and formula II-A is at least 94%.

In some embodiments, the photocleave yield of cleaving the compound of formula I-A and formula II-A is at least 96%.

In some embodiments, the photocleave yield of cleaving the compound of formula I-A and formula II-A is at least 98%.

In some embodiments, the photocleave yield of cleaving the compound of formula I-A and formula II-A is at least 99%.

In some embodiments the compound of formula I has the following structure:

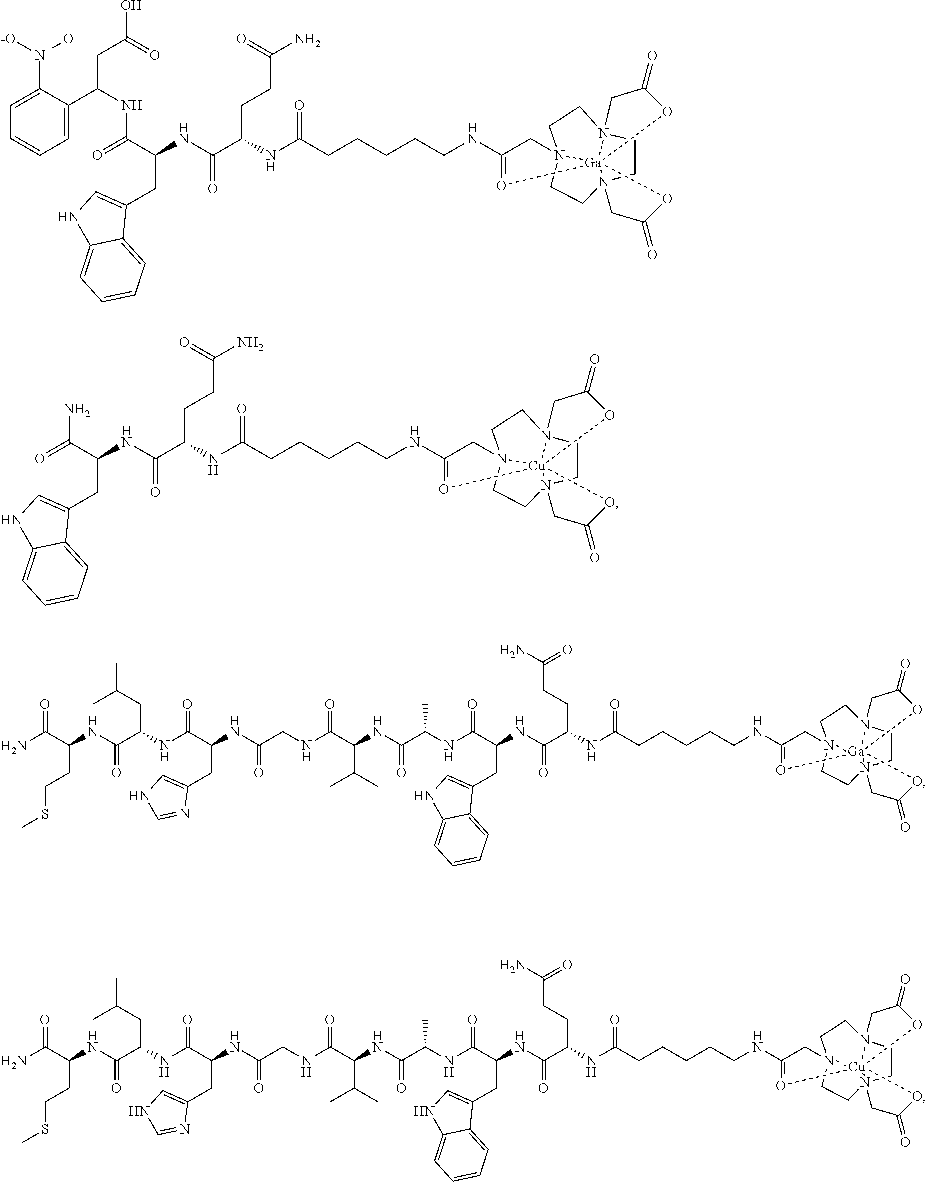

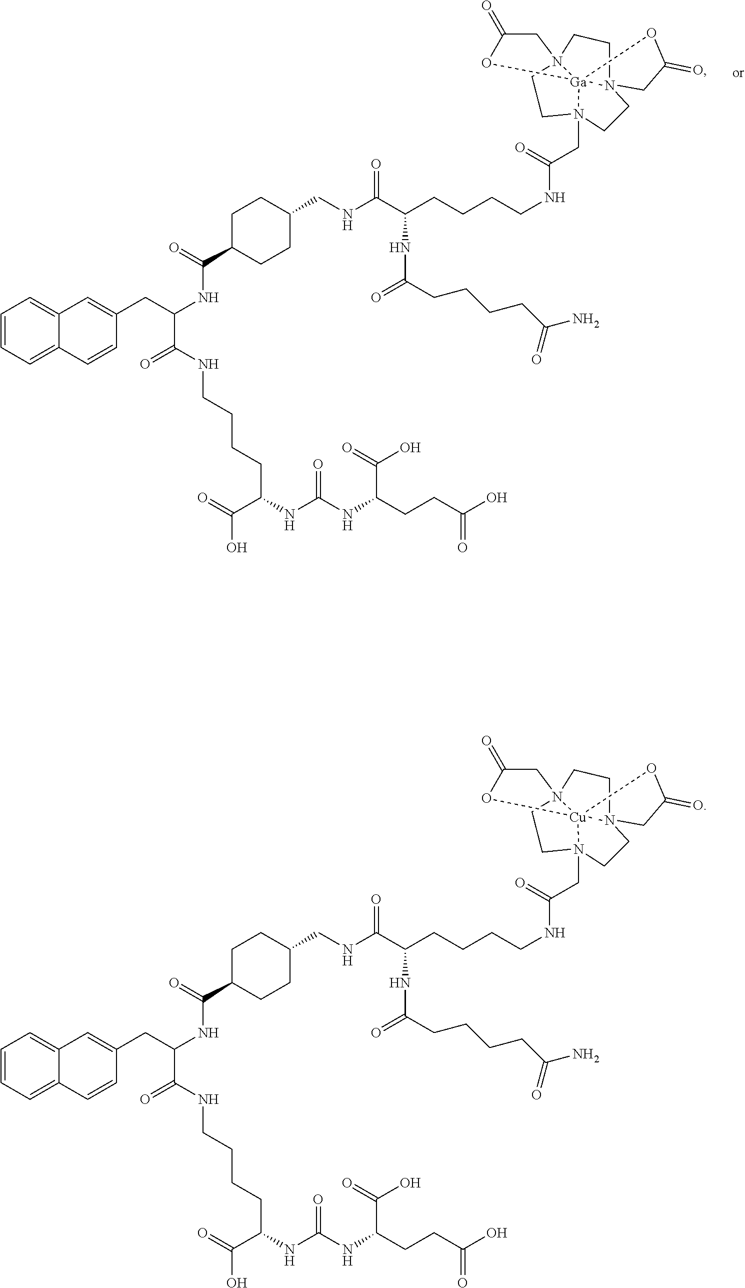

In some embodiments, the compound of formula I has the following structure:

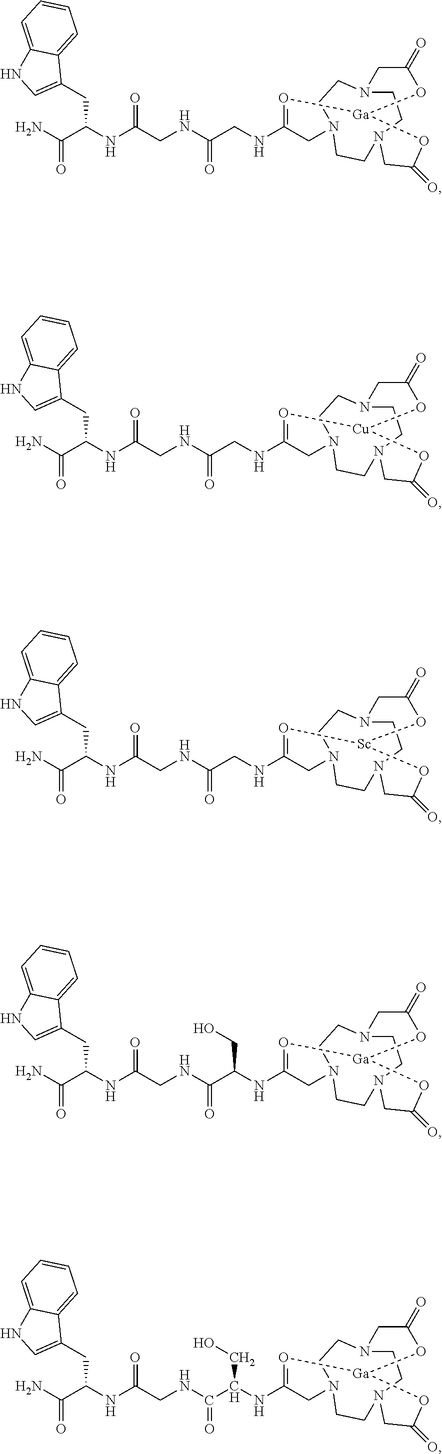

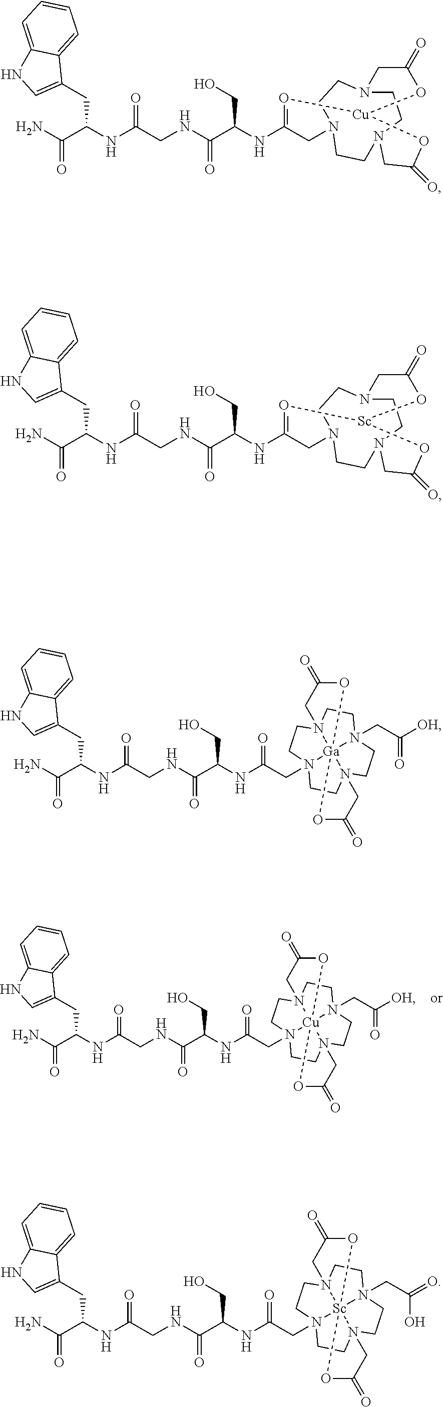

In some embodiments, the compound of formula II has the following structure:

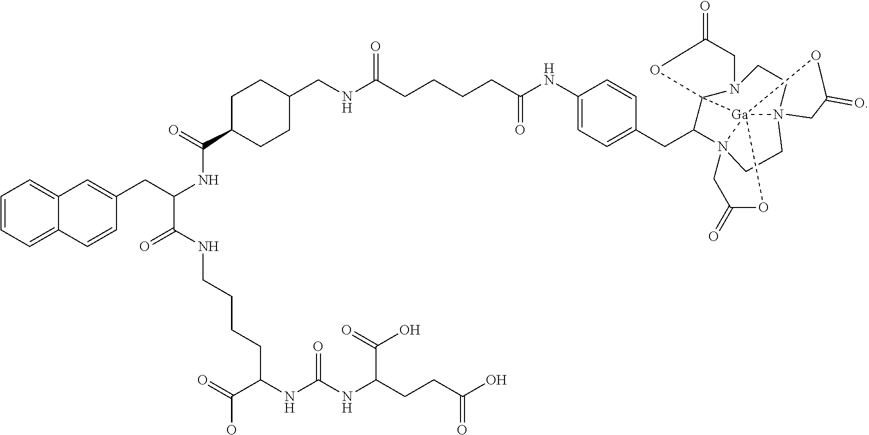

This invention provides a composition comprising a compound attached to a solid surface having the structure:

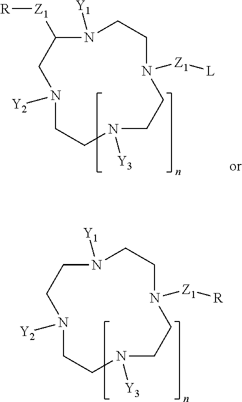

wherein
wherein R is

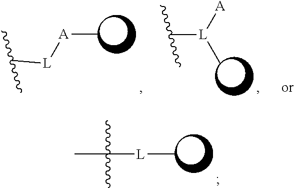

n is 0 or 1;
Y₁, Y₂, Y₃ are each, independently, —H, alkylheteroaryl, alkyl-CO₂H, alkylaryl-CO₂H, alkylheteroaryl-CO₂H, alkyl-CO₂R₄, alkylaryl-NH—CO₂P₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N(alkylaryl-CO₂P₄)₂, alkyl-N(alkylheteroaryl-CO₂P₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ and alkylheteroaryl-P(O)(OH)₂;
wherein each occurrence of R₄ is independently, —H, alkyl, alkenyl, alkynyl, alkyl-aryl, alkyl-heteroaryl, aryl, heteroaryl, alkyl-CF₃ or —Si(alkyl)₃;
Z₁ is

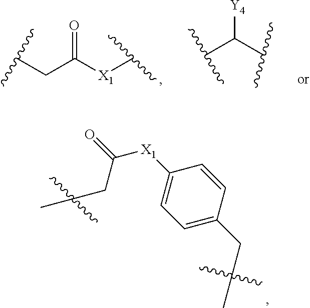

wherein X₁ is NH, O or S, and
Y₄ is —CO₂H, —CO₂R₅, aryl-CO₂H, heteroaryl-CO₂H, aryl-CO₂R₅ or heteroaryl-CO₂R₅, and wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
   L is a peptide linker;
   A is a targeting moiety or an activating group;
or a pharmaceutically acceptable salt of the compound.

In some embodiments of the composition, one of Y₁ or Y₂ is —H, or each of Y₁ and Y₂ is —H.

In some embodiments of the composition, one of Y₁ or Y₂ is alkyl-CO₂H, or each of Y₁ and Y₂ is alkyl-CO₂H.

In some embodiments of the composition, one of Y₁ or Y₂ is alkylaryl-CO₂H or alkyl-CO₂R₄, or each of Y₁ and Y₂ are alkylaryl-CO₂H or alkyl-CO₂R₄.

In some embodiments of the composition, R₄ is alkyl, alkenyl, alkynyl.

In some embodiments of the composition R₄ is alkyl, alkenyl.

In some embodiments of the composition R₄ is alkyl.

In some embodiments of the composition, R₄ is C₁-C₈ alkyl.

In some embodiments of the composition, R₄ is C₁-C₆ alkyl.

In some embodiments of the composition, R₄ is C₃-C₆ alkyl.

In some embodiments of the composition, R₄ is butyl.

In some embodiments of the composition, R₄ is tert-butyl.

In some embodiments of the composition, the peptide linker contains at least two, three, four, five or six amino acids.

In some embodiments of the composition, the peptide linker contains at least three, four, five amino acids.

In some embodiments of the composition, the peptide linker contains at least three or four amino acids.

In some embodiments of the composition, the peptide linker contains three amino acids.

In some embodiments of the composition, the peptide linker contains 6-aminohexanoic acid (Aca), 3-amino-3-(2-nitrophenyl)propionic acid (Anp) and pyroglutamic acid.

In some embodiments of the composition, the peptide linker contains amino acids selected from serine (ser/S), glycine (gly/G), tryptophan (trp/W), glutamine (gln/Q), arginine (arg/R), leucine (leu/L), asparagine (asn/N), alanine (ala/A), valine (val/V), histidine (his/H), and methionine (met/M).

In some embodiments of the composition, the peptide sequence contains amino acids selected from Q, W and G.

In some embodiments of the composition, the peptide sequence contains amino acids selected from Q and G.

In some embodiments of the composition, the peptide linker is Aca-gln-trp.

In some embodiments of the composition, the peptide linker is gln-trp-ala-val-gly-his-leu-met.

In some embodiments of the composition, the peptide linker is gly-gly-trp.

In some embodiments of the composition, the peptide linker is Aca-bombesin (BBN).

In some embodiments of the composition, the peptide linker is ser-gly-trp.

In some embodiments of the composition, the peptide linker is Aca-gln-trp-anp.

In some embodiments of the composition, A is a targeting moiety.

In some embodiments of the composition, the targeting moiety is trastuzumab, bombesin, somatostatin or 2-[3-(1,3-dicarboxypropyl)ureido]pentanedioic acid (DUPA) or a derivative or fragment thereof.

In some embodiments of the composition, A is activating group.

In some embodiments of the composition, the activating group is to activate carboxylic acids.

In some embodiments of the composition, the activating group is Bis(pentafluorophenyl) carbonate or N-Hydroxysuccinimide.

In some embodiments of the composition, R₁, R₂, R₃ are independently OH or t-butyl.

In some embodiments of the composition, R₁, R₂, R₃ are OH; or wherein R₁, R₂, R₃ are t-butyl.

In some embodiments, the composition having the structure:

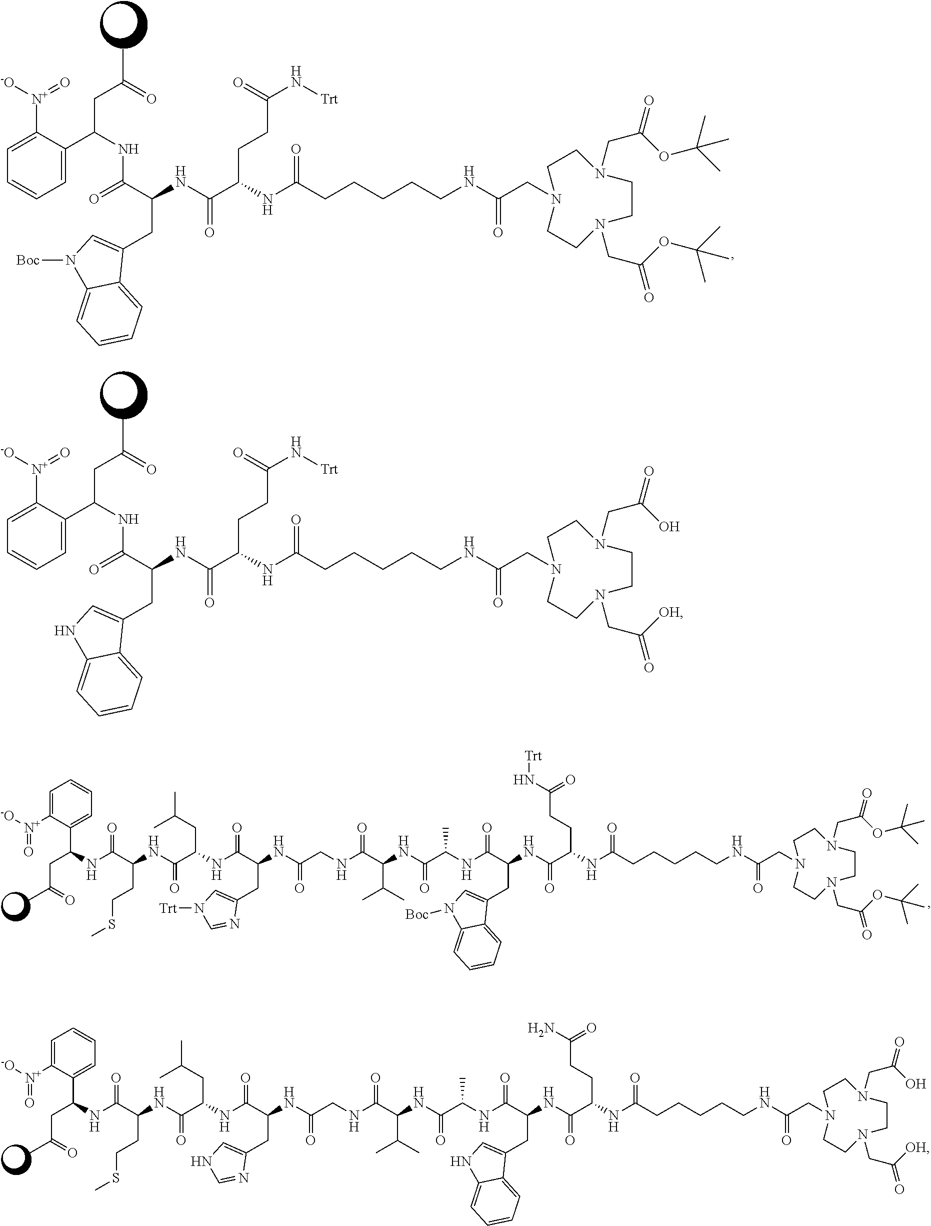

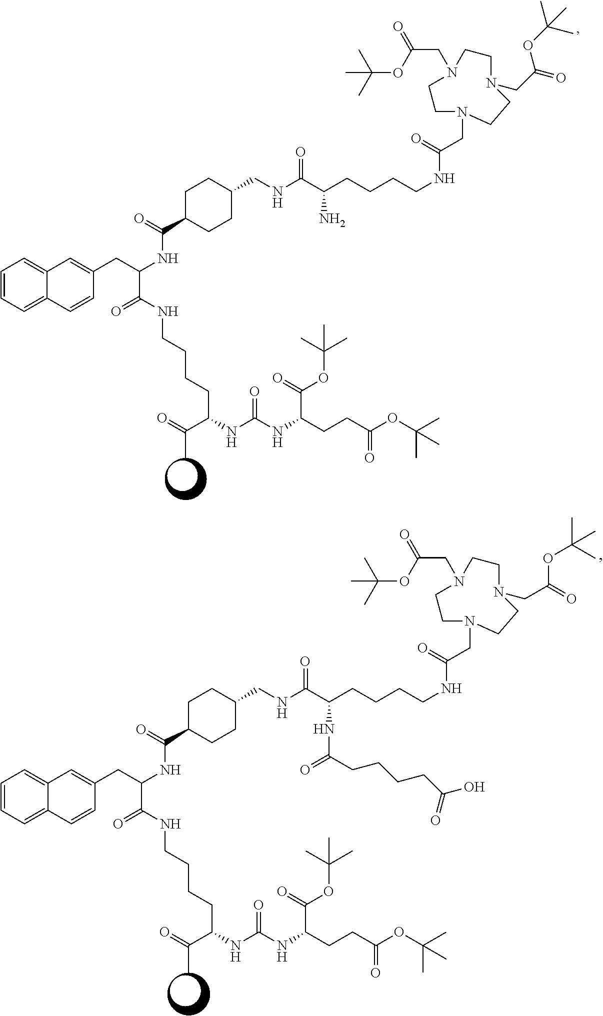 [SMILES: CC(=O)[C@H](CCCCNC(=O)C(Cc1ccc2ccccc2c1)NC(=O)[C@H]1CC[C@H](CNC(=O)[C@@H](N)CCCCNC(=O)CN2CCN(CC(=O)OC(C)(C)C)CCN(CC(=O)OC(C)(C)C)CC2)CC1)NC(=O)N[C@@H](CCC(=O)OC(C)(C)C)C(=O)OC(C)(C)C ; CC(=O)[C@H](CCCCNC(=O)C(Cc1ccc2ccccc2c1)NC(=O)[C@H]1CC[C@H](CNC(=O)[C@H](CCCCNC(=O)CN2CCN(CC(=O)OC(C)(C)C)CCN(CC(=O)OC(C)(C)C)CC2)NC(=O)CCCCC(=O)O)CC1)NC(=O)N[C@@H](CCC(=O)OC(C)(C)C)C(=O)OC(C)(C)C]

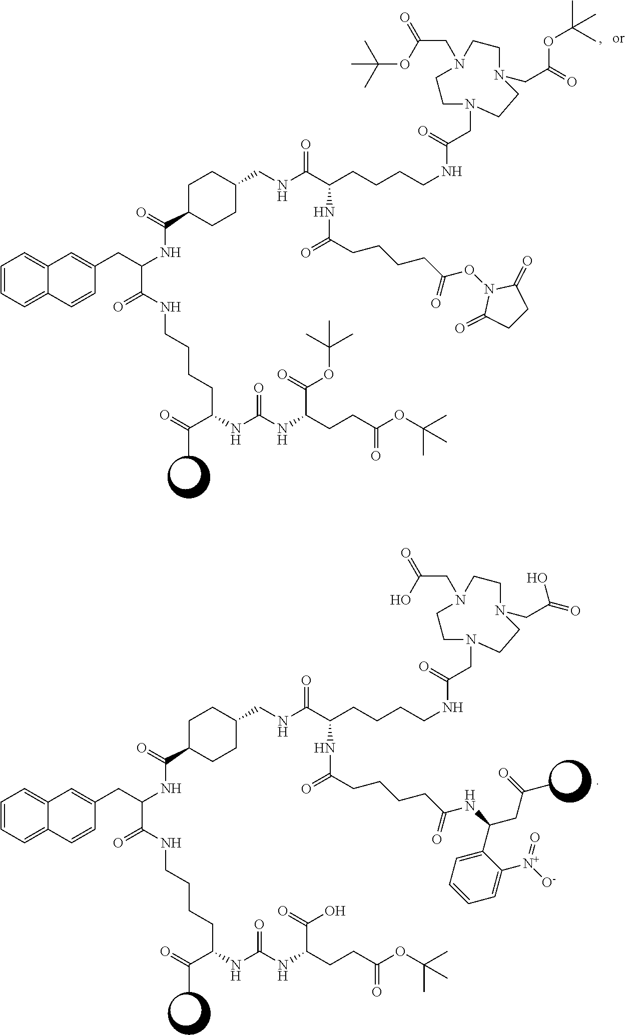 [SMILES: CC(=O)[C@H](CCCCNC(=O)C(Cc1ccc2ccccc2c1)NC(=O)[C@H]1CC[C@H](CNC(=O)[C@H](CCCCNC(=O)CN2CCN(CC(=O)OC(C)(C)C)CCN(CC(=O)OC(C)(C)C)CC2)NC(=O)CCCCC(=O)ON2C(=O)CCC2=O)CC1)NC(=O)N[C@@H](CCC(=O)OC(C)(C)C)C(=O)OC(C)(C)C ; CC(=O)C[C@H](NC(=O)CCCCC(=O)N[C@@H](CCCCNC(=O)CN1CCN(CC(=O)O)CCN(CC(=O)O)CC1)C(=O)NC[C@H]1CC[C@H](C(=O)NC(Cc2ccc3ccccc3c2)C(=O)NCCCC[C@H](NC(=O)N[C@@H](CCC(=O)OC(C)(C)C)C(=O)O)C(C)=O)CC1)c1ccccc1[N+](=O)[O-]]

In some embodiments, the composition having the structure:

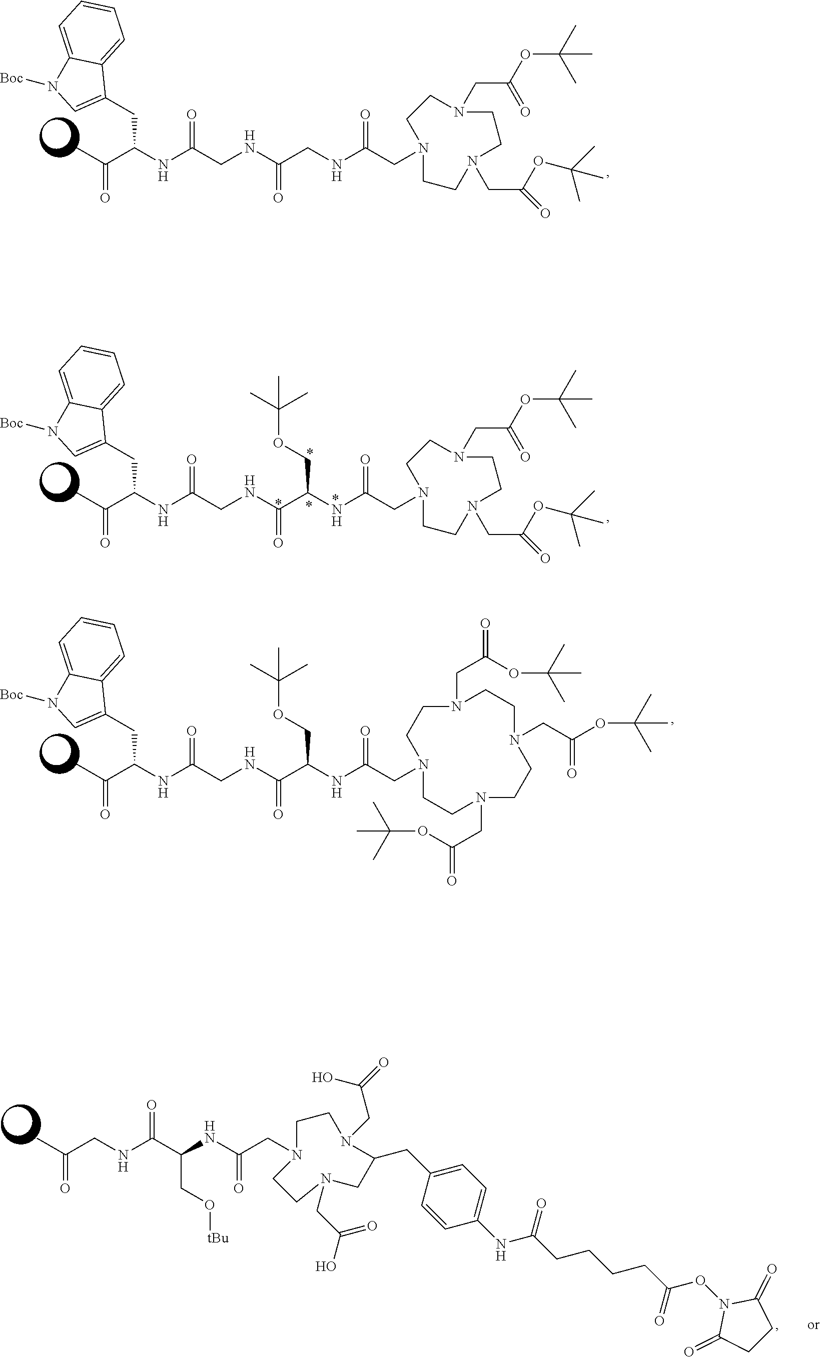

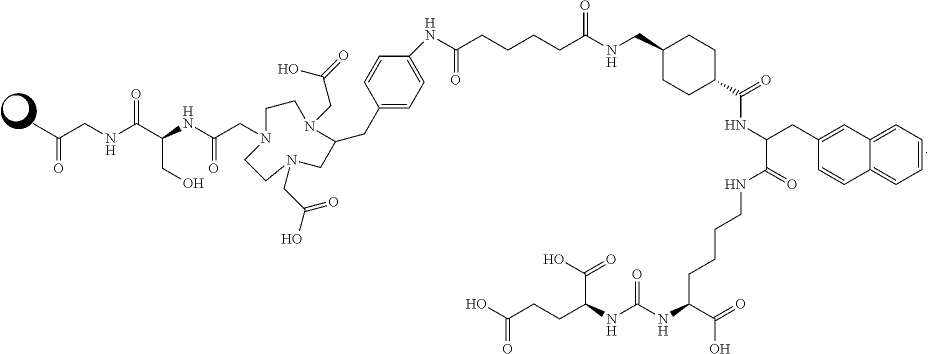 [SMILES: CC(=O)CNC(=O)[C@H](CO)NC(=O)CN1CCN(CC(=O)O)CC(Cc2ccc(NC(=O)CCCCC(=O)NC[C@H]3CC[C@H](C(=O)NC(Cc4ccc5ccccc5c4)C(=O)NCCCC[C@H](NC(=O)N[C@@H](CCC(=O)O)C(=O)O)C(=O)O)CC3)cc2)N(CC(=O)O)CC1]

In some embodiments, the composition further coordinates with a metal ion to form a metal complex.

In some embodiments, the metal ion is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb); preferably, the metal is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (44Sc), Scandium-47 (⁴⁷Sc), or Scandium-43 (⁴³Sc); more preferably, the metal is Gallium-67 (Ga⁶⁷), Gallium-68 (Ga⁶⁸), Copper-62 (⁶²Cu), Copper-64 (⁶⁴Cu), or Copper-67 (⁶⁷Cu).

The present invention provides a method of detecting target cells in a subject comprising administering an effective amount of the metal complex disclosed in the current invention to the subject, and imaging the subject with a molecular imaging device to detect the metal complex or composition in the subject.

In some embodiments of the method, the target cells are cancer cells.

The present invention provides a method of imaging target cells in a subject comprising:
a) administering to the subject an effective amount of the metal complex disclosed in the current invention or a pharmaceutically acceptable salt thereof, wherein the metal complex or the composition specifically accumulates at the target cells in the subject;
b) detecting in the subject the location of the metal complex or the composition; and
c) obtaining an image of the target cells in the subject based on the location of the metal complex or the composition in the subject.

The present invention provides a method of detecting the presence of target cells in a subject which comprises determining if an amount of the metal complex disclosed in the current invention or a pharmaceutically acceptable salt thereof, is present in the subject at a period of time after administration of the metal complex or composition to the subject, thereby detecting the presence of the target cells based on the amount of the metal complex or composition determined to be present in the subject.

In some embodiments of the method, the detection is performed by a Positron Emission Tomography (PET) device.

The present invention provides a method of detecting cancer cells in a subject comprising administering an effective amount of the metal complex of disclosed in the current invention, and imaging the subject with a molecular imaging device to detect the metal complex or composition in the subject, wherein the cancer cells are prostate cancer cells, wherein the cancer cells have elevated levels of prostate-specific membrane antigen (PSMA).

The present invention provides a method of imaging prostate cancer cells in a subject comprising:
a) administering to the subject an effective amount of the metal complex disclosed in the current invention or a pharmaceutically acceptable salt thereof, wherein the metal complex or the composition specifically accumulates at prostate cancer cells in the subject;
b) detecting in the subject the location of the metal complex or the composition; and
c) obtaining an image of the cancer cells in the subject based on the location of the metal complex or the composition in the subject.

The present invention provides a method of detecting the presence of prostate cancer cells in a subject which comprises determining if an amount of the metal complex disclosed in the current invention or a pharmaceutically acceptable salt thereof, is present in the subject at a period of time after administration of the metal complex or composition to the subject, thereby detecting the presence of the prostate cancer cells based on the amount of the metal complex or composition determined to be present in the subject.

In some embodiments of the method, the metal complex is further prepared by the following steps:
a) conjugate the metal complex to a solid support surface;
b) transport the metal complex and the solid support surface to an imaging device; and
c) photo cleave the metal complex.

In some embodiments of the method, the metal complex is photo cleaved at a wavelength about 300-400 nm.

In some embodiments of the method, the metal complex is photo cleaved at a wavelength about 320-370 nm.

In some embodiments of the method, the metal complex is photo cleaved at a wavelength at 365 nm.

The present invention provides a method of reducing the size of a prostate tumor or of inhibiting proliferation of prostate cancer cells comprising contacting the tumor or cancer cells with the metal complex disclosed in the current invention or a pharmaceutically acceptable salt thereof, so as to thereby reducing the size of the tumor or inhibit proliferation of the cancer cells.

In some embodiments, Y₁ and Y₂ are each independently, —H, alkyl-CO₂H, alkylaryl-CO₂H, alkylheteroaryl-CO₂H, alkyl-CO₂R₄, alkylaryl-CO₂R₄, alkylheteroaryl-CO₂R₄, preferably, Y₁ and Y₂ are each independently-H, alkyl-CO₂H, alkylaryl-CO₂H, alkylheteroaryl-CO₂H; more preferably, Y₁ and Y₂ are each independently —H and alkyl-CO₂H.

In some embodiments, the peptide sequence contains three amino acids.

In some embodiments, the peptide sequence contains four amino acids.

In some embodiments, the peptide sequence contains five amino acids.

In some embodiments, the peptide sequence contains at least one peptide linker group.

In some embodiments, the peptide sequence contains two peptide linker groups.

In some embodiments, the peptide sequence contains does not contain a peptide linker group.

In some embodiments, the peptide linker group is selected from 6-aminohexanoic acid (Aca), 3-amino-3-(2-nitrophenyl)propionic acid (Anp) and pyroglutamic acid.

In some embodiments, the peptide sequence contains amino acids selected from serine (ser/S), glycine (gly/G), tryptophan (trp/W), glutamine (gln/Q), arginine (arg/R), leucine (leu/L), asparagine (asn/N), alanine (ala/A), valine (val/V), histidine (his/H), and methionine (met/M).

In some embodiments, the peptide sequence contains amino acids selected from Q, W and G.

In some embodiments, more preferably, peptide sequence contains amino acids selected from Q and G.

In some embodiments, the peptide sequence is Aca-gln-trp, gln-trp-ala-val-gly-his-leu-met, gly-gly-trp, ser-gly-trp or Aca-gln-trp-anp.

In some embodiments, the metal is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵Tb) or Terbium-161 (¹⁶¹Th) In some embodiments, the metal is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), or Scandium-43 (⁴³Sc).

In some embodiments, the metal is Gallium-67 (Ga⁶⁷), Gallium-68 (Ga⁶⁸), Copper-62 (⁶²Cu), Copper-64 (⁶⁴Cu), or Copper-67 (⁶⁷Cu).

In some embodiments, the resin is a rink amide resin, a wang resin, an agarose resin, a tentagel resin or an ion-exchange resin.

In some embodiments, the resin is rink amide resin.

In some embodiments, the resin was swollen for 3 times at an interval of 3 minutes.

In some embodiments, the resin was swollen for 3 times at an interval of 1 minute.

In some embodiments, 100-200 mg of Fmoc-Trp(Boc)-OH at a concentration of 0.29 mmol was used.

In some embodiments, 153 mg of Fmoc-Trp(Boc)-OH was used.

In some embodiments, 50-150 mg PyBOP at a concentration of 0.19 mmol was used.

In some embodiments, 100 mg PyBOP was used.

In some embodiments, 1-100 μL DIEA at a concentration of 0.38 mmol was used.

In some embodiments, 65 μL DIEA was used.

In some embodiments, the NOTA derivative is NOTA-bis(t-Bu ester).

In some embodiments, the DOTA derivative is DOTA-tris(t-Bu ester).

Each embodiment disclosed herein is contemplated as being applicable to each of the other disclosed embodiments. Thus, all combinations of the various elements described herein are within the scope of the invention. In addition, the elements recited in the composition embodiments can be used in the method and use embodiments described herein and vice versa.

The present invention is illustrated and further described in more detail with reference to the following non-limiting examples. The following examples illustrate the practice of the present subject matter in some of its embodiments but should not be construed as limiting the scope of the present subject matter. Other embodiments will be apparent to one skilled in the art from consideration of the specification and examples. It is intended that the specification, including the examples, is considered exemplary only without limiting the scope and spirit of the present subject matter.

All combinations of the various elements disclosed herein are within the scope of the invention.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

This invention will be better understood by reference to the Examples which follow, but those skilled in the art will readily appreciate that the specific experiments detailed are only illustrative of the invention as described more fully in the claims which follow thereafter

Other methods of producing the compound of Formula I and the compound of Formula II are also described in U.S. application Ser. No. 17/253,307, and PCT Application No. PCT/US 22/78389, filed Oct. 19, 2022, the contents of which are hereby incorporated by reference.

U.S. application Ser. No. 17/253,307 and PCT Application No. PCT/US 22/78389 described alternative methods for preparing the compound of Formula I and the compound of Formula II.

### Definitions

Unless defined otherwise, all technical and scientific terms used herein have the meaning commonly understood by persons of ordinary skill in the art to which this subject matter pertains.

The term “a” or “an”, as used herein, includes the singular and the plural, unless specifically stated otherwise. Therefore, the terms “a,” “an,” or “at least one” can be used interchangeably in this application.

As used herein, the term “about” when used in connection with a numerical value includes +10% from the indicated value. In addition, all ranges directed to the same component or property herein are inclusive of the endpoints, are independently combinable, and include all intermediate points and ranges. It is understood that where a parameter range is provided, all integers within that range, and tenths thereof, are also provided by the invention. For example, “30-45%” includes 30%, 30.1%, 30.2%, etc. up to 45%.

As used herein, the term “amino acid” refers to any natural or unnatural amino acid including its salt form, ester derivative, protected amine derivative and/or its isomeric forms. Amino Acids comprise, by way of non-limiting example: Agmatine, Alanine Beta-Alanine, Arginine, Asparagine, Aspartic Acid, Cysteine, Glutamine, Glutamic Acid, Glycine, Histidine, Isoleucine, Leucine, Lysine, Methionine, Phenylalanine, Phenyl Beta-Alanine, Proline, Serine, Threonine, Tryptophan, Tyrosine, and Valine. The amino acids may be L or D amino acids.

The terms “peptide”, “polypeptide”, peptidomimetic and “protein” are used to refer to a polymer of amino acid residues. The terms apply to amino acid polymers in which one or more amino acid residues is an artificial chemical mimetic of a corresponding naturally occurring amino acid, as well as to naturally occurring amino acid polymers. These terms also encompass the term “antibody”. “Peptide” is often used to refer to polymers of fewer amino acid residues than “polypeptides” or “proteins”. A protein can contain two or more polypeptides, which may be the same or different from one another.

As used herein, the term “oligopeptide” refers to a peptide comprising of between 2 and 20 amino acids and includes dipeptides, tripeptides, tetrapeptides, pentapeptides, etc.

An amino acid or oligopeptide may be covalently bonded to an amine of another molecule through an amide linkage, resulting in the loss of an “OH” from the amino acid or oligopeptide.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As sued herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons and five hydrogens.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric coated to prevent release of the active ingredient compounds before they reach the small intestine. Materials that may be used as enteric coatings include, but are not limited to, sugars, fatty acids, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

The compounds of the present invention can be synthesized according to general Schemes. Variations on the following general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

While the invention has been shown and described with reference to certain embodiments of the present invention thereof, it will be understood by those skilled in the art that various changes in from and details may be made therein without departing from the spirit and scope of the present invention and equivalents thereof.

### EXPERIMENTATION

### Example 1

Materials and Methods. All starting materials were purchased from Acros Organics, Alfa Aesar, Millipore Sigma, TCI America and used without further purification. Fmoc-protected amino acids were purchased from Bachem. NOTA-bis(t-Bu ester) compound was obtained from Macrocyclics. Agarose beads, amine functional, were purchased from NANOCS company. AmberLite® FPA66 Anion Exchange Resin free base and TentaGel™ S—NH2 resins were purchased from Millipore Sigma. ⁶⁷Ga-citrate was received from Jubilant Radiopharma company as ⁶⁷Ga(citrate). ⁶⁴CuCl₂ isotope was produced and received from the University of Wisconsin, Medical Physics Dept., Madison, WI. Mass spectrometry: High-resolution ESI mass spectrometry was carried out at the Stony Brook University Center for Advanced Study of Drug Action (CASDA) mass spectrometry facility with an Agilent LC-UV-TOF spectrometer. MALDI-TOF MS and high-resolution (ESI) mass spectrometry was carried out at the Stony Brook University Institute for Chemical Biology and Drug Discovery (ICB&DD) Mass Spectrometry Facility with an Agilent LC/MSD and Agilent LC-UV-TOF spectrometers, respectively. Inductively coupled plasma spectroscopy (ICP) was performed on an Agilent Technologies ICP-OES (Model 5110). A 10-point standard with respect to gallium or copper was used and lines of best fit were found with R₂ of 0.999. UV-vis spectra were collected with a NanoDrop 1 C instrument (AZY1706045).

High-Performance Liquid Chromatography (HPLC): Semi-Preparative HPLC was carried out using a Shimadzu HPLC-20AR equipped with a binary gradient, pump, UV-vis detector, and manual injector on a Phenomenex Luna C18 column (250 mm×21.2 mm, 100 Å, AXIA packed). Method A (Preparative Purification Method). A=0.1% TFA in water, B=0.1% TFA in MeCN. Gradient: 0-5 min: 95% A; 5-24 min: 5-95% B gradient. Analytical HPLC analysis was carried out using a Shimadzu HPLC-20AR equipped with a binary gradient, pump, UV-vis detector, autoinjector, and Laura radio-detector on a Phenomenex Luna C18 column (150 mm×3 mm, 100 Å). Method B (Analytical HPLC analysis). A=0.1% TFA in water, B=0.10% TFA in MeCN with a flow rate of 0.8 mL/min, UV detection at 220 and 270 nm. Gradient 0-2 min: 5% B; 2-14 min 5-95% B; 14-16 min 95% B; 16-16.5 min 95-5% B; 16.5-20 min 5% B. Radio-HPLC analysis was carried out using a Shimadzu HPLC-20AR equipped with a binary gradient, pump, UV-vis detector, autoinjector, and Laura radio-detector on a Phenomenex Luna C18 column (150 mm×3 mm, 100 Å). Method C (Radioanalysis). A=0.1% TFA in water, B=0.1% TFA in MeCN with a flow rate of 0.8 mL/min. Gradient 0-2 min: 5% B; 2-14 min 5-95% B; 14-16 min 95% B; 16-16.5 min 95-5% B; 16.5-20 min 5% B.

### I. Synthesis and Characterization of Peptides

### Synthesis of BBN

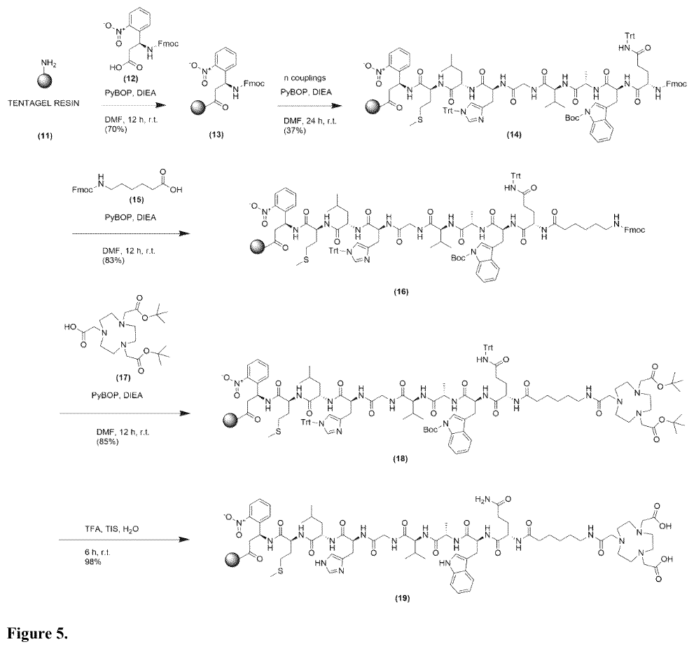

The synthesis of bombesin (BBN) peptide (14) was carried out on a 0.05 mmol scale using a TENTAGEL resin (200 mg, 0.26 mmol/g). (Abd-Elgaliel, 2007). The resin (11) was swollen in DCM (3 mL) and DMF (3 mL) for 1 min three times each. (S)-3-(Fmoc-amino)-3-(2-nitrophenyl)propionic acid (2) (68 mg, 0.15 mmol) was coupled to N terminus of the resin using Benzotriazole-1-yl-oxy-tris-pyrrolidino-phosphonium hexafluorophosphate (PyBOP) (54 mg, 0.11 mmol) as coupling reagent in the presence of N,N Diisopropylethylamine (DIEA) (36 μL, 0.21 mmol) within 12 h. The Fmoc group was subsequently removed by treatment of the resin with 20% piperidine in DMF (3 mL) for 20 min. Subsequent amino acids were coupled in the same manner until the full sequence (GNQWAVGHLM) was assembled (see FIG. 5). Subsequently, the Fmoc group on N-terminus was deprotected and Fmoc-6-Ahx-OH (74 mg, 0.21 mmol) was coupled using PyBOP (54 mg, 0.11 mmol) as coupling reagent in the presence of DIEA (36 μL, 0.21 mmol) within 12 h. Finally, the Fmoc group on N-terminus was deprotected and NOTA-bis(t-Bu ester) (65 mg, 0.15 mmol) was coupled using PyBOP (166 mg, 0.32 mmol) as coupling reagent in the presence of DIEA (115 μL, 0.64 mmol) within 6 h. Eventually, the resin was washed, dried and stored until use.

### Synthesis of PMSA

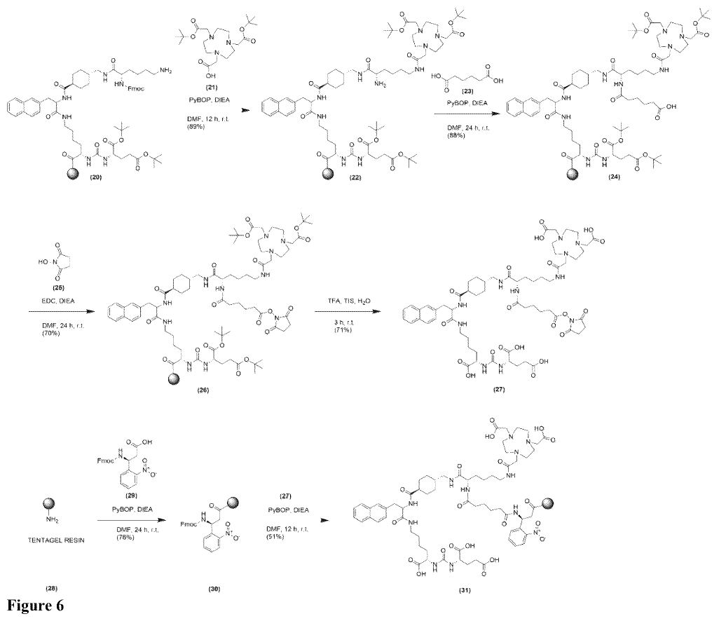

The synthesis of the PSMA-targeting peptide (31, Glu-urea-Lys-nap-trans-Lys-NOTAADPA-NH2) was carried out on a 0.16 mmol scale using a Wang resin 200 mg, 0.8 mmol/g) (see FIG. 6). The starting material, Glu-urea-Lys-Nap-trans-Lys, was synthesized according previously established protocol. (Vaughn, 2021). The Lys(Mtt)-trans-nap-Lys-urea-Glu-Wang resin (200 mg, 0.16 mmol) was treated with a 1% TFA in DCM to selectively remove the Mtt protecting group. Next, NOTA-bis(t-Bu ester) (199 mg, 0.48 mmol) was coupled to the free amine of the Lys side-chain using PyBOP (166 mg, 0.32 mmol) as coupling reagent in the presence of DIEA (115 μL, 0.64 mmol) within 6 h. The Fmoc group from N-terminus was removed by treatment the resin with solution of 20% piperidine in DMF for 20 min. The peptide was elongated by coupling the adipic acid using PyBOP (166 mg, 0.32 mmol) as the coupling reagent in the presence of DIEA (94 mg, 115 μL, 0.64 mmol) within 12 h.

Subsequently, the NHS active ester of PSMA-targeting peptide (compound 7) was synthesized by addition of excess of NHS (1104 mg, 8.0 mmol) was used to obtain the active ester of resin-bound peptide by using EDC (1.522 g, 8.0 mmol) as the coupling reagent in the presence of DIEA (1.39 mL, 8.00 mmol) within 16 h. After coupling, all resin was washed, dried and treated with a mixture of TFA/TIS/H2O (95%/2.5%/2.5%) to cleave compound 7 from the resin. Compound 7 was purified by reverse-phase semi-preparative HPLC and subsequently re-appended to H-Anp-TENTAGEL resin. To this end, TENTAGEL resin (200 mg, 0.26 mmol/g) was swollen in DCM and DMF. Next, Fmoc-Anp-OH (67 mg, 0.16 mmol) was coupled to resin using PyBOP (54 mg, 0.10 mmol) as coupling reagent in the presence of DIEA (36 L, 0.21 mmol) within 12 h. The Fmoc group was removed by treatment the resin with solution of 20% piperidine in DMF for 25 min. Subsequently, compound 7 (26 mg, 0.02 mmol) was coupled to the preloaded H-Anp-TENTAGEL resin in the presence of DIEA (18 μL, 0.1 mmol) within 6 h.

### BBN Deprotection

More commonly used deprotection mixtures such as TFA/TIS/H₂O causes methionine oxidation to sulfoxide in the side chain of the BBN sequence; accordingly, formation of 15% of BBN peptide with a C-terminal methionine oxide side-product was observed under these conditions. (Hackenberger, 2006) The addition of 1,2-ethanethiol (EDT) to the cleavage cocktail (reagent K), reduced the presence of methionine-oxidized analogue to 5% (see Table 1). To prevent the formation of oxidized methionine residues in peptide sequence, the cleavage cocktail containing an NH4I-Me2S (ammonium iodide and dimethylsulfide) was used for acidic deprotection of side chains and terminal cleavage from the resin support. Using the NH4I-Me2S containing cleavage cocktail H for the acidic deprotection, resulted in only 1% of oxidized side product. To fully eliminate the presence of methionine sulfoxide on the resin support, the amount of NH4I:Me2S reductant pair was increased to 5:10%. Under these conditions, following HPLC analysis, no presence of methionine sulfoxide-peptide was detected.

### Quantitation of Loading Efficiency, Deprotection and Terminal Cleavage

### Approximation of Resin Loading.

To determine the loading of the resin with respect to peptide conjugation, a 5.0 mg of appropriate dry resin was weighed, transferred to a plastic syringe equipped with a filter membrane, and pre-swollen in DCM and DMF. Fmoc-deprotection was carried out using standard procedure. All solution was collected through filtration and diluted in ratio 1:25. The UV absorbance was measured at 301 nm in a quartz cuvette with a 1 cm path length to determine the resin loading using the standard formula as the following:

$$Resin loading = \frac{A ( 301 ) \times V \times d}{ε \times W \times m}$$

where A(301)=UV absorbance at 301 nm, V=cleavage volume (2 mL), d=dilution factor (25), c=molar extinction coefficient of Fmoc group (7800 mL mmol-1 cm-1), W=cuvette width (1 cm), and m=resin mass in g. (Lee, 2018) Fmoc deprotection.

Fmoc deprotection was performed twice by treating the Fmoc protected peptide with 2 mL of 20% piperidine in DMF (each time 15 min). For the synthesis of all compounds, the N-terminal Fmoc group was deprotected, and after each step the resin was washed with DMF (5×2 mL). TFA cleavage. The resin was suspended in a mixture of TFA/TIS/H₂O (2 mL; 95:2.5:2.5, v/v/v) at room temperature for 3 h. Then the resin was removed and the residual TFA solution transferred to a 15 mL falcon tube and an ice-cold mixture of Et₂O/hexane (10 mL; 1:1 v/v) was added to precipitate the peptide. The supernatant was removed after centrifugation, and the peptide pellets were washed twice with Et₂O/hexane. The crude peptide precipitate was re-dissolved in MeCN/H₂O (1:1, v/v) and lyophilized, followed by purification by semi-preparative HPLC. Pure fractions were collected and lyophilized characterized by analytical HPLC and MALDI-TOF MS.

### II. Synthesis and Characterization of Coordination Complexes

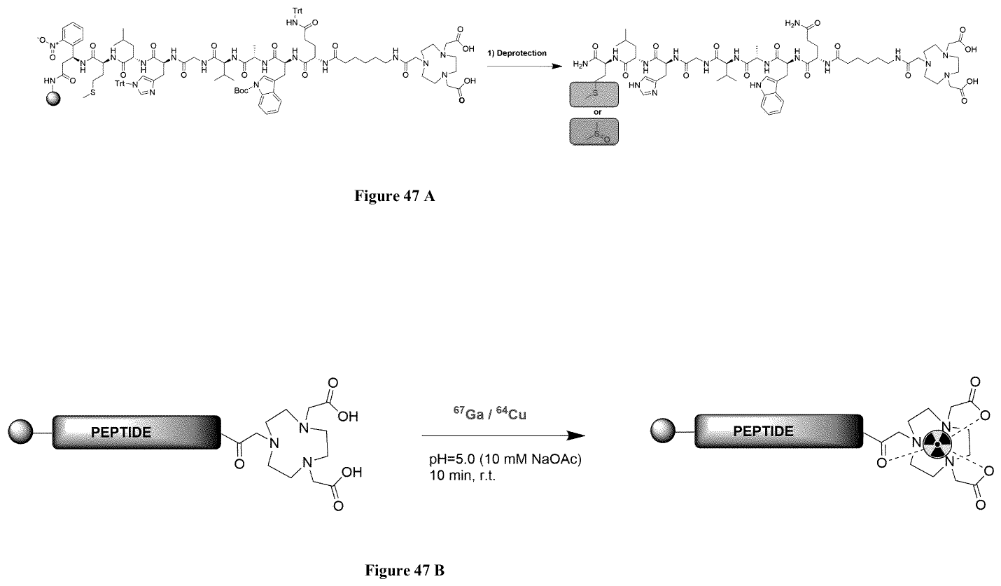

TABLE 1
Reagents and conditions for NOTA-ACA-BBN deprotection. (See FIG. 47 A)

| | | Time | Side- | Nota-Aca- |
|---|---|---|---|---|
| Entry | Deprotection mixture | [h] | product | BBN % |
| 1 | TFA/TIS/H2O | 1 | 15 | 85 |
| | (95%/2.5%/2.5%) | | | |
| 2 | TFA/TIS/H2O | 3 | 15 | 85 |
| | (95%/2.5%/2.5%) | | | |
| 3 | TFA/TIS/EDT/H2O | 1 | 15 | 85 |
| | (90%/2.5%/5.0%/2.5%) | | | |
| 4 | TFA/TIS/EDT/H2O | 3 | 15 | 85 |
| | (90%/2.5%/5.0%/2.5%) | | | |
| 5 | TFA/TIS/TA/(CH3)2S/NH4I/H2O | 1 | 5 | 95 |
| | (80%/5.0%/5.0%/5.0%/2.5%/2.5%) | | | |
| 6 | TFA/TIS/TA/(CH3)2S/NH4I/H2O | 3 | 5 | 95 |
| | (80%/5.0%/5.0%/5.0%/2.5%/2.5%) | | | |
| 7 | TFA/Phenol/TA/EDT/(CH3)2S/NH4I/H2O | 1 | 1 | 99 |
| | (80%/2.5%/5.0%/2.5%/5.0%/2.5%/2.5%) | | | |
| 8 | TFA/Phenol/TA/EDT/(CH3)2S/NH4I/H2O | 3 | 1 | 99 |
| | (80%/2.5%/5.0%/2.5%/5.0%/2.5%/2.5%) | | | |
| 9 | TFA/Phenol/TA/EDT/(CH3)2S/NH4I/H2O | 1 | — | 100 |
| | (70%/5.0%/5.0%/2.5%/10%/5.0%/2.5%) | | | |
| 10 | TFA/Phenol/TA/EDT/(CH3)2S/NH4I/H2O | 3 | — | 100 |
| | (70%/5.0%/5.0%/2.5%/10%/5.0%/2.5%) | | | |

### Photocleavage

The appropriate Anp-linked resin was suspended in 10% Ethanol in H₂O in a filter frit endowed plastic syringe. The syringe was subsequently placed inside the photoreactor and reacted under gentle agitation and cooling with the built-in fan and photoirradiation with the 360 nm LED light source. After photocleavage, the product was filtered and characterized by HPLC (or radio-HPLC) HR-ESI-MS or MALDI-TOF MS.

### III. Synthesis and Characterization of Coordination Complexes

The resin-appended peptide, NOTA-ACA-BBN (1.0 mg, 100 nmol) or NOTA-PSMA (4.0 mg, 100 nmol), was washed with 3 mL of DCM, DMF, H2O and 10 mM NaOAc buffer (pH=5.0) three times each for 1 min. Next, the aqueous stock solutions of Ga³⁺ or Cu²⁺ were prepared and an aliquot of the resin corresponding to one equivalent was added and reacted with the peptide appended resin for at room temperature for 12 h in pH 5 NaOAc buffer to achieve full complexation. The metal-peptide conjugates were subsequently photocleaved and the formation of complexed molecule was affirmed with analytical HPLC and mass spectrometry (ESI-MS and MALDI-TOF). The complex conjugates were then purified via semi-preparative HPLC.

TABLE 2
Radiolabeling reaction and data. (See FIG. 47 B)

| Peptide | Radiometal | SA [μCi/nmol] | RCY [%] | RCP [%] |
|---|---|---|---|---|
| BBN (19) | ⁶⁷Ga | 5.0 | 99.9 | 99.9 |
| PSMA (31) | ⁶⁷Ga | 5.0 | 99.9 | 99.9 |
| BBN (19) | ⁶⁴Cu | 5.1 | 99.8 | 99.8 |
| PSMA (31) | ⁶⁴Cu | 5.1 | 99.8 | 99.8 |

### IV. General Radiolabeling Protocol

### Resin Preparation

The resin-bound peptide, NOTA-ACA-BBN (0.1 mg, 10 nmol) or NOTA-PSMA (0.4 mg, 10 nmol), was washed with 3 mL of DCM, DMF, H2O and 10 mM NaOAc buffer (pH=5.0) three times each for 1 min. Eventually, the resin was stored in 10 mM of sodium acetate (NaOAc) buffer until use.

Radiolabeling with ⁶⁷Ga.

⁶⁷Ga-citrate was purchased from Jubilant Radiopharma at an average specific activity of 185.0 MBq/mL. The ⁶⁷Ga-citrate solution was first converted to ⁶⁷GaCl₃ using an established solid-phase extraction protocol (Pandey, 2021). The resulting average specific activity of the obtained 67Ga-chloride solution used for solid-phase radio-labelling was 40.7 MBq/mL For radio-labelling of NOTA-Aca-BBN or NOTA-PSMA, a 10 μL aliquot containing 2.1 MBq of ⁶⁷GaCl₃ was added to the resin-bound conjugate (10 nmol, 300 L) in 10 mM of sodium acetate (NaOAc) buffer. The pH of the solution was adjusted to 5.0. Radiolabeling was complete after 20 minutes at room temperature. The radiolabeled conjugates were photocleaved for 10 min and characterized with radio-HPLC. ⁶⁷Ga-NOTA-Aca-BBN: Rₜ=7.13 min (purity/peak area: 99%). ⁶⁷Ga-NOTAPSMA: Rₜ=7.43 min (purity/peak area: 99%).

Radiolabeling with ⁶⁴Cu.

The ⁶⁴Cu isotope stock solution (copper-64 chloride, pH=2) was produced at the University of Wisconsin-Madison, Medical Physics Dept., Madison, WI. The ⁶⁴Cu chloride was obtained at an average specific activity of 8.5 MBq/μL. For radiolabeling of NOTA-Aca-BBN or NOTA-PSMA, a 15 μL aliquot containing 1.85 MBq of ⁶⁴CuCl₂ was added to the resin-bound ligand (10 nmol, 300 L) in 10 mM of sodium acetate (NaOAc) buffer. The pH of the solution was adjusted to 5.0. Radiolabeling was complete within 20 minutes at room temperature. The radiolabeled conjugates were photocleaved for 10 min and characterized with radio-HPLC. ⁶⁴Cu-NOTA-Aca-BBN: Rt=7.42 min (purity/peak area: 99%).

Synthesis of ⁶⁷Ga-radiolabeled complexes Radiolabeling with ⁶⁷Ga. The resin-bound peptide, NOTA-Aca-Q-W-NH₂ (0.1 mg, 10 nmol) was washed with 3 mL of DCM, DMF, H2O and 10 mM NaOAc buffer (pH=5.0) three times each for 1 min. Eventually, the resin was stored in 10 mM of sodium acetate (NaOAc) buffer until use. ⁶⁷Ga-citrate was purchased from Jubilant Radiopharma at an average specific activity of 185.0 MBq/mL. The ⁶⁷Ga-citrate solution was first converted to ⁶⁷GaCl₃ using an established solid-phase extraction protocol. The resulting average specific activity of the obtained ⁶⁷Ga-chloride solution used for solid-phase radio-labelling was 40.7 MBq/mL. For radio-labelling of NOTA-Aca-Q-W-NH₂, a 10 μL aliquot containing 2.1 MBq of ⁶⁷GaCl₃ was added to the resin-bound conjugate (10 nmol, 300 L) in 10 mM of sodium acetate (NaOAc) buffer. The pH of the solution was adjusted to 5.0. Radiolabeling was complete after 20 minutes at room temperature. The radiolabeled conjugates were photocleaved for 10 min and characterized with radio-HPLC. ⁶⁷Ga-NOTAAca-Q-W-NH₂: Rt=6.20 min (purity/peak area: 99%).

⁶⁷Gallium 2,2′-(7-((5S,8S)-5-((1H-indol-3-yl)methyl)-8-(3-amino-3-oxopropyl)-1-carboxy-2-(2-nitrophenyl)-4,7,10,17-tetraoxo-3,6,9,16-tetraazaoctadecan-18-yl)-1,4,7-triazonane-1,4-diyl)diacetate (10F). Compound 10F was synthesized using the general radiolabeling protocol followed by photocleavage from intermediate 10E. The product was isolated by direct filtration-mediated removal of the resin support and characterized using radio-HPLC chromatography. Rₜ (Method C): 6.20 min. SA: 5.0 μCi/nmol, RCY: 99%, RCP: 99%.

⁶⁷Gallium 2,2′-(7-((5S,8S,11S,17S,20S,23S,26S)-11-((1H-imidazol-4-yl)methyl)-23-((1H-indol-3-yl)methyl)-26-(3-amino-3-oxopropyl)-5-carbamoyl-8-isobutyl-17-isopropyl-20-methyl-7,10,13,16,19,22,25,28,35-nonaoxo-2-thia-6,9,12,15,18,21,24,27,34-nonaazahexatriacontan-36-yl)-1,4,7-triazonane-1,4-diyl)acetate (20F). Compound 20F was synthesized using the general radio labeling protocol followed by photocleavage from intermediate 20E. The product was isolated by direct filtration-mediated removal of the resin support and characterized using radio-HPLC chromatography. Rₜ (Method C): 7.28 min. SA: 5.0 μCi/nmol, RCY: 99.9%, RCP: 99.9%.

⁶⁷Gallium of (((1S)-5-(2-((1S,4S)-4-(((S)-2-(6-amino-6-oxohexanamido)-6-(2-(4,7-bis(carboxymethyl)-1,4,7-triazonan-1-yl)acetamido)hexanamido)methyl)cyclohexane-1-carboxamido)-3-(naphthalen-2-yl)propanamido)-1-carboxypentyl)carbamoyl)-L-glutamic acid bisacetate (30F). Compound 30F was synthesized using the general radiolabeling protocol followed by photocleavage from intermediate 30E. The product was isolated by direct filtration-mediated removal of the resin support and characterized using radio-HPLC chromatography. Rₜ (Method C): 7.43 min. SA: 5.1 μCi/nmol, RCY: 99%, RCP: 99%.

Gallium 2,2′-(7-((5S,8S)-5-((1H-indol-3-yl)methyl)-8-(3-amino-3-oxopropyl)-1-carboxy-2-(2-nitrophenyl)-4,7,10,17-tetraoxo-3,6,9,16-tetraazaoctadecan-18-yl)-1,4,7-triazonane-1,4-diyl)diacetate (10B). Compound 10B was synthesized using the generalized complexation procedure outlined above. A followed by photocleavage from compound 10A. The product was isolated as a pale yellow solid following deprotection and characterized using mass spectrometry and HPLC chromatography. Yield: (32 nmol, 32%). Rt (Method A): 7.62 min. MALDI-MS [M]+ calc. for C43H56GaN10O13 989.3384, found 989.445.

### Synthesis of ⁶⁴Cu-Radiolabeled Complexes

⁶⁴Copper 2,2′-(7-((5S,8S,11S,7S,20S,23S,26S)-11-((1H-imidazol-4-yl)methyl)-23-((1H-indol-3-yl)methyl)-26-(3-amino-3-oxopropyl)-5-carbamoyl-8-isobutyl-17-isopropyl-20-methyl-7,10,13,16,19,22,25,28,35-nonaoxo-2-thia-6,9,12,15,18,21,24,27,34-nonaazahexatriacontan-36-yl)-1,4,7-triazonane-1,4-diyl)diaceticate (20H). Compound 20H was synthesized using general radiolabeling protocol followed by photocleavage from intermediate 20G. The product was isolated by direct filtration-mediated removal of the resin support and characterized using radio-HPLC chromatography. Rₜ (Method C): 7.42 min. SA: 5.0 μCi/nmol, RCY: 99%, RCP: 99%.

⁶⁴Copper (((1S)-5-(2-((1S,4S)-4-(((S)-2-(6-amino-6-oxohexanamido)-6-(2-(4,7-bis(carboxymethyl)-1,4,7-triazonan-1-yl)acetamido)hexanamido)methyl)cyclohexane-1-carboxamido)-3-(naphthalen-2-yl)propanamido)-1-carboxypentyl)carbamoyl)-L-glutamic acid diacetate (30H). Compound 30H was synthesized using general radiolabeling protocol followed by photocleavage strategy from intermediate 30G. The product was isolated by direct filtration-mediated removal of the resin support and characterized using radio-HPLC chromatography. Rₜ (Method C): 7.65 min. SA: 5.1 μCi/nmol, RCY: 99%. RCP: 99%.

Copper 2,2′-(7-((5S,8S)-5-((1H-indol-3-yl)methyl)-8-(3-amino-3-oxopropyl)-1-carboxy-2-(2-nitrophenyl)-4,7,10,17-tetraoxo-3,6,9,16-tetraazaoctadecan-18-yl)-1,4,7-triazonane-1,4-diyl)diacetate, (10D). Compound 10D was synthesized using the generalized complexation procedure outlined above followed by photocleavage from compound 10C. The product was isolated as a pale blue solid following deprotection and characterized using mass spectrometry and HPLC chromatography. Yield: (30 nmol, 30%). Rₜ (Method A): 6.12 min. MALDI-MS [M+H]+ calc. for C34H49CuN9O9 791.3027, found 791.333.

Synthesis of NOTA-Aca-Q-W-NH₂ model peptide. The synthesis of the NOTA-Aca-Q-W-NH₂peptide (10) was carried out on a 0.2 mmol scale using a 2-Chlorotrityl chloride resin (200 mg, 1.0 mmol/g). The resin (1) was swollen in DCM (3 mL) and DMF (3 mL) for 1 min three times each. (S)-3-(Fmoc-amino)-3-(2-nitrophenyl)propionic acid (2) (312 mg, 0.60 mmol) was coupled to the resin using N,N-Diisopropylethylamine (DIEA) (139 μL, 0.80 mmol) in DCM within 12 h. The Fmoc group was subsequently removed by treatment of the resin with 20% piperidine in DMF (3 mL) for 20 min. Subsequent amino acids were coupled in the same manner until the short sequence (QW) was assembled. The peptide was elongated by coupling the Fmoc-Aca-OH (283 mg, 0.80 mmol) using PyBOP (208 mg, 0.40 mmol) as the coupling reagent in the presence of DIEA (139 μL, 0.80 mmol) within 12 h.

The Fmoc group from N-terminus was removed by treatment the resin with solution of 20% piperidine in DMF for 20 min. Next, NOTA-bis(t-Bu ester) (166 mg, 0.40 mmol) was coupled to the free amine of the Lys side-chain using PyBOP (208 mg, 0.40 mmol) as coupling reagent in the presence of DIEA (139 μL, 0.80 mmol) within 6 h. The resin-appended protected NOTA-Aca-Q-W-Anp peptide (200 mg, 0.20 mmol) was treated with a 1% TFA in DCM to selectively cleave the peptide from solid phase. Subsequently, the NHS active ester of NOTA-Aca-Q-W-Anp (compound 7) was synthesized by addition of excess of NHS (460 mg, 4.0 mmol) and EDC (621 mg, 4.0 mmol) as the coupling reagent in the presence of DIEA (1.40 mL, 8.00 mmol) within 16 h. After coupling, the activated peptide was treated with a mixture of TFA/TIS/H2O (95%/2.5%/2.5%) to deprotect the side chain of the peptide. Compound 7 was purified by reverse-phase semi-preparative HPLC and subsequently re-appended to tentagel resin, agarose-based resin or ion-exchange resin. To this end, each resin (100 mg, 0.26 mmol/g) was swollen in DCM and DMF.

Next, the compound 7 (27 mg, 0.03 mmol) was coupled to three types of resin using DIEA (18 μL, 0.11 mmol) in DCM within 12 h. Eventually, the resin was washed, dried and stored until use. Fmoc deprotection. Fmoc deprotection was performed twice by treating the Fmoc protected peptide with 2 mL of 20% piperidine in DMF (each time 15 min). For the synthesis of all compounds, the N-terminal Fmoc group was deprotected, and after each step the resin was washed with DMF (5×2 mL). TFA cleavage. The resin was suspended in a mixture of TFA/TIS/H₂O (2 mL; 95:2.5:2.5, v/v/v) at room temperature for 3 h. Then the resin was removed and the residual TFA solution transferred to a 15 mL falcon tube and an ice-cold mixture of Et₂O/hexane (10 mL; 1:1 v/v) was added to precipitate the peptide. The supernatant was removed after centrifugation, and the peptide pellets were washed twice with Et₂O/hexane. The crude peptide precipitate was redissolved in MeCN/H₂O (1:1, v/v) and lyophilized, followed by purification by semi-preparative HPLC. Pure fractions were collected and lyophilized characterized by analytical HPLC and MALDI-TOF MS.

(11S,14S)-1-(4,7-bis(2-(tert-butoxy)-2-oxoethyl)-1,4,7-triazonan-1-yl)-14-((1-(tert-butoxycarbonyl)-1H-indol-3-yl)methyl)-17-(2-nitrophenyl)-2,9,12,15-tetraoxo-11-(3-oxo-3-(tritylamino)propyl)-3,10,13,16-tetraazanonadecan-19-oic acid, (5). The intermediate 5 (50 mg, 0.12 mmol) was synthesized using standard SPPS strategy from compound 1 using procedures outlined above. The product was isolated as a colorless solid following deprotection and characterized using mass spectrometry and HPLC chromatography. Yield: (0.023 g, 80%). Rₜ (Method A): 13.4 min. HR-ESI-MS [M+H]+ calc. for C75H96N10O15 1377.7134, found 1377.7129.

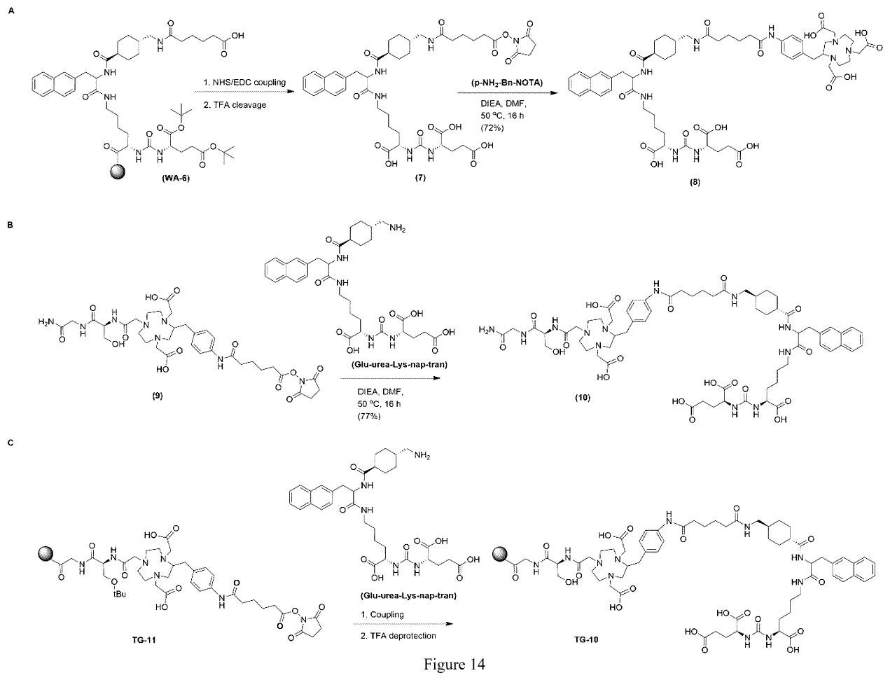

Di-tert-butyl2,2′-(7-((11S,14S)-14-((1-(tert-butoxycarbonyl)-1H-indol-3-yl)methyl)-19-((2,5-ioxopyrrolidin-1-yl)oxy)-17-(2-nitrophenyl)-2,9,12,15,19-pentaoxo-11-(3-oxo-3-(tritylamino)propyl)-3,10,13,16-tetraazanonadecyl)-1,4,7-triazonane-1,4-diyl)diacetate,(7). Intermediate 7 (50 mg, 0.12 mmol) was synthesized using NHS/EDC coupling strategy from compound 5 outlined in FIG. 14. The product was isolated as a colorless solid following deprotection and characterized using mass spectrometry and HPLC chromatography. Yield: (0.025 g, 85%). Rₜ (Method A): 13.7 min. HR-ESI-MS [M+H]+ calc. for C79H99N11O17 1474.7298, found 1474.7290.

2,2′-(7-((11S,14S)-14-((1H-indol-3-yl)methyl)-11-(3-amino-3-oxopropyl)-19-((2,5-dioxopyrrolidin-1-yl)oxy)-17-(2-nitrophenyl)-2,9,12,15,19-pentaoxo-3,10,13,16-tetraazanonadecyl)-1,4,7-triazonane-1,4-diyl)diacetic acid, (8). Intermediate 8 (50 mg, 0.12 mmol) was synthesized using standard deprotection strategy outlined above from compound 7. The product was isolated as a colorless solid following deprotection and characterized using mass spectrometry and HPLC chromatography. Yield: (0.011 g, 60%). Rₜ (Method A): 8.27 min. HR-ESI-MS [M+H]+ calc. for C47H61N11O15 1020.4426, found 1020.4413.

2,2′-(7-(2-((6-(((S)-5-amino-1-(((S)-1-amino-3-(1H-indol-3-yl)-1-oxopropan-2-yl)amino)-1,5-dioxopentan-2-yl)amino)-6-oxohexyl)amino)-2-oxoethyl)-1,4,7-triazonane-1,4-diyl)diacetic acid, (10). Compound 10 (50 mg, 0.12 mmol) was synthesized using standard SPPS strategy followed by photocleavage outlined above from compound 8. The product was isolated as a colorless solid following deprotection and characterized using mass spectrometry and HPLC chromatography. Yield: (0.009 g, 40%). Rₜ (Method A): 6.47 min. HR-ESI-MS [M+H]+ calc. for C34H51N9O9 730.3888, found 730.3881. Synthesis of complexes with Ga and Cu. The resin-appended peptide, NOTA-Aca-Q-W-NH₂ (1.0 mg, 100 nmol) was washed with 3 mL of DCM, DMF, H₂O and 10 mM NaOAc buffer (pH=5.0) three times each for 1 min. Next, the aqueous stock solutions of Ga³⁺ or Cu²⁺ were prepared and an aliquot of the resin corresponding to one equivalent was added and reacted with the peptide appended resin for at room temperature for 12 h in pH 5 NaOAc buffer to achieve full complexation. The metal-peptide conjugates were subsequently photocleaved and the formation of complexed molecule was affirmed with analytical HPLC and mass spectrometry (ESI-MS and MALDI-TOF). The complex conjugates were then purified via semi-preparative HPLC.

### Example 2

### Experimental Procedures

### Materials

All starting materials were purchased from Acros Organics, Alfa Aesar, Millipore Sigma or TCI America and used without further purification. Fmoc-protected amino acids were purchased from Bachem. NOTA-bis(t-Bu ester), DOTA-tris(t-Bu ester) and p-NH₂-Bn-NOTA compounds were obtained from Macrocyclics. Rink Amide resin, Wang resin and TentaGel™ S-NH₂ resins were purchased from Millipore Sigma. ⁶⁷Ga-citrate was received from Jubilant Radiopharma. ⁶⁸GaCl₃ was obtained from a ⁶⁸Ge/⁶⁸Ga generator (Eckhard & Ziegler, 30 mCi).

### I. General Methods of Characterization

Mass spectrometry: High-resolution ESI mass spectrometry was carried out at the Stony Brook University Center for Advanced Study of Drug Action (CASDA) mass spectrometry facility with an Agilent LC-UV-TOF spectrometer. MALDI-TOF MS and high-resolution (ESI) mass spectrometry was carried out at the Stony Brook University Institute for Chemical Biology and Drug Discovery (ICB&DD) Mass Spectrometry Facility with an Agilent LC/MSD and Agilent LC-UV-TOF spectrometers, respectively. NMR spectra (¹H, ¹³C) were collected on a 700 MHz Advance III Bruker, 500 MHz, or 400 MHz Bruker instrument at 25° C. and processed using TopSpin 4.0.7. Chemical shifts are reported as parts per million (ppm).

Inductively coupled plasma spectroscopy (ICP) was performed on an Agilent Technologies ICP-OES (Model 5110). A 10-point standard with respect to gallium or copper was used and lines of best fit were found with R² of 0.999. UV-vis spectra were collected with a NanoDrop 1 C instrument (AZY1706045).

High-Performance Liquid Chromatography (HPLC): Semi-Preparative HPLC was carried out using a Shimadzu HPLC-20AR equipped with a binary gradient, pump, UV-vis detector, and manual injector on a Phenomenex Luna C18 column (250 mm×21.2 mm, 100 Å, AXIA packed).

Method A (Preparative Purification Method). A=0.1% TFA in water, B=0.1% TFA in MeCN. Gradient: 0-5 min: 95% A; 5-24 min: 5-95% B gradient. Analytical HPLC analysis was carried out using a Shimadzu HPLC-20AR equipped with a binary gradient, pump, UV-vis detector, autoinjector, and Laura radio-detector on a Phenomenex Luna C18 column (150 mm×3 mm, 100 Å).

Method B (Analytical HPLC analysis). A=0.1% TFA in water, B=0.1% TFA in MeCN with a flow rate of 0.8 mL/min, UV detection at 220 and 270 nm. Gradient 0-2 min: 5% B; 2-14 min 5-95% B; 14-16 min 95% B; 16-16.5 min 95-5% B; 16.5-20 min 5% B.

Method C (Analytical HPLC analysis). A=0.1% FA in water, B=0.1% FA in MeCN with a flow rate of 0.8 mL/min, UV detection at 220 and 270 nm. Gradient 0-3 min: 5% B; 3-10 min 5-95% B; 10-13 min 95% B; 13-13.5 min 95-5% B; 13.5-16 min 5% B. Radio-HPLC analysis was carried out using a Shimadzu HPLC-20AR equipped with a binary gradient, pump, UV-vis detector, autoinjector, and Laura radio-detector on a Phenomenex Luna C18 column (150 mm×3 mm, 100 Å).

Method D (Radioanalysis). A=0.1% TFA in water, B=0.1% TFA in MeCN with a flow rate of 0.8 mL/min. Gradient 0-2 min: 5% B; 2-14 min 5-95% B; 14-16 min 95% B; 16-16.5 min 95-5% B; 16.5-20 min 5% B.

Method E (LC-MS analysis). A=0.1% FA in water, B=0.1% FA in MeCN with a flow rate of 0.8 mL/min, UV detection at 220 and 270 nm. Gradient 0-3 min: 5% B; 3-10 min 5-95% B; 10-13 min 95% B; 13-13.5 min 95-5% B; 13.5-16 min 5% B.

Method F (LC-MS analysis). A=0.1% NH₄CH₃CO₂ in water, B=0.1% NH₄CH₃CO₂ in MeCN with a flow rate of 0.8 mL/min, UV detection at 220 and 270 nm. Gradient 0-3 min: 5% B; 3-10 min 5-95% B; 10-13 min 95% B; 13-13.5 min 95-5% B; 13.5-16 min 5% B.

VTMS and CIVP: All experiments were carried out in a home-built quadrupole time-of-flight mass spectrometer described elsewhere (Waller, 2019). Briefly, the complexes were introduced into the gas phase via an electrospray ionization (ESI) source purged with dry air using PEEK tubing of 50 μm inner diameter. All samples were diluted to 50-100 μM in 50/50 acetonitrile/water from 500 μM stock solutions and an optimized flow rate of 0.6 μL/min was employed. The ESI voltages were typically 2050-2080 V, low enough to prevent sample degradation while still ensuring steady signal. Once generated, the ions were introduced into a vacuum system and stored in a room temperature 81ropenam trap. Ions were extracted from this trap and mass selected by a quadrupole mass filter (Extrel), then guided into a cryogenically cooled ion trap capable of operating in a temperature range of 310 K-3 K. For Variable temperature mass spectrometry (VTMS) measurements (Racow, 2019), a mixture of water and helium was introduced via a pulsed valve allowing the formation of hydrated complexes, if thermodynamically feasible. A fraction of ions was then extracted, at 10 Hz, into a reflectron time-of-flight (TOF) mass spectrometer to analyze the relative intensities of the resulting hydrates. The trap was cooled down from 310 K at a rate of ˜1 K/min and mass spectra were collected by summing 1000 scans at 10 K intervals in the cooling down period. The partial pressure of water was maintained between 4×10−8 to 2×10⁻⁷ Torr as detected by a residual gas analyzer (Extorr). Vibrational spectra were recorded using a method known as Cryogenic Ion Vibrational Predissociation (CIVP) spectroscopy as described previously (Yang 2019). Briefly, a mixture of N₂ and helium was seeded into the cryogenic ion trap, encouraging the formation of nitrogen adducts (so-called “tags”) typically between 45 K-48 K. Tagged complexes were extracted into the TOF and intersected by a tunable infrared laser pulse from a Nd:YAG pumped OPO/OPA system (LaserVision). The tagged ions absorb a single photon when the wavelength is resonant with a vibrational transition, resulting in the desorption of the tag from the ions. Fragment ion yields were subsequently quantified by a reflectron mass spectrometer. The ratio of fragment to tagged ions, corrected for the laser power, at each wavelength thus yields a linear absorption spectrum of the ions of interest that can be analyzed in the same way as atypical FTIR spectrum.

### II. Synthesis and Characterization of Peptides

### a. Synthesis of Model Tripeptides

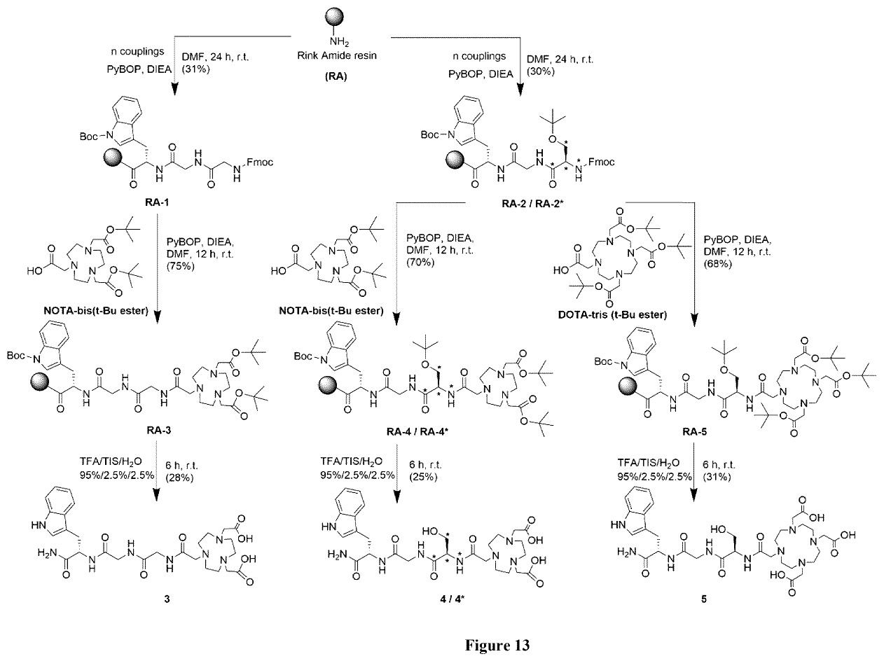

The synthesis of model tripeptides Gly-Gly-Trp (1) and Ser-Gly-Trp (2) was carried out on a 0.093 mmol scale using a Rink Amide (RA) resin (150 mg, 0.62 mmol/g). The RA resin was swollen in DCM (3 mL) and DMF (3 mL) for 1 min three times each. Fmoc-Trp(Boc)-OH (153 mg, 0.29 mmol) was loaded onto the resin using Benzotriazole-1-yl-oxy-tris-pyrrolidino-phosphonium hexafluorophosphate (PyBOP) (100 mg, 0.19 mmol) as coupling reagent in the presence of N,N-Diisopropylethylamine (DIEA) (65 μL, 0.38 mmol) within 12 h. The standard capping step has been carried out using acetic anhydride/pyridine mixture (3:2) for 30 min. The Fmoc group was subsequently removed by treatment of the resin with 20% piperidine in DMF (3 mL) for 20 min. Subsequent amino acids were coupled in the same manner until the full sequences were assembled giving RA-1 (Gly-Gly-Trp) and RA-2 (Ser-Gly-Trp) (see FIG. 13). Finally, the Fmoc group on N-terminus was deprotected and NOTA-bis(t-Bu ester) (120 mg, 0.29 mmol) was coupled using PyBOP (100 mg, 0.19 mmol) as coupling reagent in the presence of DIEA (65 μL, 0.38 mmol) within 12 h resulting in RA-3 and RA-4. Additionally, DOTA-tris(t-Bu ester) (162 mg, 0.29 mmol) was coupled to RA-2 using PyBOP (100 mg, 0.19 mmol) as coupling reagent in the presence of DIEA (65 μL, 0.38 mmol) within 12 h resulting in RA-5. Eventually, the products RA-3, RA-4, and RA-5 were washed, dried, and stored until use. In order to release the final model tripeptides from the solid support, the appropriate resin-appended ligands were treated with a mixture of TFA/TIS/H₂O (95%/2.5%/2.5%).

### b. Synthesis of PSMA Peptides

The synthesis of the PSMA-targeting peptide (8, Glu-urea-Lys-nap-trans-ADPA-Bn-NOTA) was carried out on a 0.16 mmol scale using a Wang resin (200 mg, 0.8 mmol/g) (see FIG. 14A). The starting material, Glu-urea-Lys-Nap-trans, was synthesized according previously established protocol (Smilowicz, 2022). The Fmoc group from N-terminus was removed by shaking the resin with solution of 20% piperidine in DMF for 20 min. The peptide was elongated (WA-6) by coupling the adipic acid using PyBOP (166 mg, 0.32 mmol) as the coupling reagent in the presence of DIEA (115 μL, 0.64 mmol) within 12 h. Subsequently, the NHS active ester of PSMA-targeting peptide (WA-7) was synthesized by addition of excess of NHS (1104 mg, 8.0 mmol) and EDC (1.522 g, 8.0 mmol) as the coupling reagent in the presence of DIEA (1.39 mL, 8.00 mmol) within 16 h. After coupling, all resin was washed, dried and treated with a mixture of TFA/TIS/H₂O (95%/2.5%/2.5%) to cleave compound 7 from the resin. Compound 7 (61.3 mg, 0.07 mmol) was purified by reverse-phase semi-preparative HPLC and the coupling to the p-NH₂-Bn-NOTA (18 mg, 0.04 mmol) was carried out in the presence of DIEA (24 μL, 0.14 mmol) at 50° C. within 16 h resulting in compound 8. In order to obtain the auto-cleavable Ser-containing construct (10), the coupling between the pre-activated Gly-Ser-NOTA-Bn-ADPA-NHS molecule and the Glu-urea-Lys-Nap-trans targeting sequence was carried out in the presence of DIEA (202 μL, 1.16 mmol) at 50° C. within 16 h (see FIG. 14B). Finally, the same auto-cleavable Ser-containing construct was prepared on tentagel resin (TG-10). The Glu-urea-Lys-Nap-trans targeting sequence (19 mg, 0.03 mmol) was re-appended to TG-11 resin (50 mg, 0.26 mmol/g) in the presence of DIEA (226 μL, 1.30 mmol) at 50° C. within 16 h (see FIG. 14C). After coupling, the tentagel resin was washed, dried and treated with a mixture of TFA/TIS/H₂O (95%/2.5%/2.5%) for 1 h to remove the side-chain protecting group, resulting in resin-appended conjugate TG-10.

### c. HRMS, HR-ESI-MS and HPLC Characterization Data

(S)-2-(2-(2-aminoacetamido)acetamido)-3-(1H-indol-3-yl)83ropenamide, (1). Compound 1 (50 mg, 0.031 mmol) was synthesized using the general coupling strategy outlined in Section II(a) and FIG. 13 from compound RA. The product was isolated as a colorless solid following deprotection and purification using Method A and characterized using mass spectrometry and HPLC chromatography. Yield: (3.5 mg, 0.011 mmol, 36%). Rₜ (Method B): 5.91 min. ¹H NMR (400 MHz, D₂O): δ 7.7 (d, 1H), 7.5 (d, 1H), 7.3 (t, 2H), 7.2 (t, 1H), 4.7 (t, 1H), 3.9 (d, 2H), 3.78 (s, 2H), 3.37-3.32 (m, 2H). ¹³C NMR (100 MHz, D₂O): δ 176.13 (C¹⁰), 170.83 (C¹⁴), 167.60 (C¹²), 136.02 (C¹), 126.87 (C⁶), 124.53 (C⁷), 121.92 (C³), 119.33 (C⁴), 118.32 (C⁵), 111.87 (C²), 108.90 (C⁸), 54.16 (C¹¹), 42.14 (C¹³), 40.27 (C¹⁵), 27.00 (C⁹). HR-ESI-MS [M+H]⁺ calc. For C₁₅H₁₉N₅O₃ 317.148790, found 318.1557.

(R)-2-amino-N-(2-(((S)-1-amino-3-(1H-indol-3-yl)-1-oxopropan-2-yl)amino)-2-oxoethyl)-3-hydroxypropanamide, (2). Compound 2 (50 mg, 0.031 mmol) was synthesized using the general coupling strategy outlined in Section II(a) and FIG. 13 from compound RA. The product was isolated as a colorless solid following deprotection and purification using Method A and characterized using mass spectrometry and HPLC chromatography. Yield: (3.9 mg, 0.011 mmol, 36%). Rₜ (Method B): 5.91 min. ¹H NMR (400 MHz, D₂O): δ 7.7 (d, 1H), 7.5 (d, 1H), 7.2 (d, 2H), 7.1 (d, 1H), 4.67 (s, 1H), 4.12 (s, 1H), 3.99-3.87 (m, 4H), 3.34-3.21 (m, 2H). ¹³C NMR (100 MHz, D₂O): δ 176.12 (C¹⁰), 170.66 (C¹⁴), 168.31 (C¹²), 136.10 (C¹), 126.85 (C⁶), 124.52 (C⁷), 121.93 (C³), 119.33 (C⁴), 118.32 (C⁵), 111.87 (C²), 108.90 (C⁸), 59.99 (C¹⁶), 54.50 (C¹¹), 54.16 (C¹⁵), 42.25 (C¹³), 27.04 (C⁹). HR-ESI-MS [M+H]⁺ calc. For C₁₆H₂₁N₅O₄ 347.159355, found 348.1660.

(R)-2-(amino-¹⁵N)—N-(2-(((S)-1-amino-3-(1H-indol-3-yl)-1-oxopropan-2-yl)amino)-2-oxoethyl)-3-hydroxypropanamide-1,2, 3-¹³C₃, (2). Compound 2* (50 mg, 0.031 mmol) was synthesized using the general coupling strategy outlined in Section II(a) and FIG. 13 from compound RA. The product was isolated as a white solid following deprotection and purification using Method A and characterized using mass spectrometry and HPLC chromatography. Yield: (5.7 mg, 0.016 mmol, 52%). Rₜ (Method B): 5.90 min. HR-ESI-MS [M+H]⁺ calc. for [¹³C]₃[¹⁵N]₁C₁₃H₂₂N₄O₄ 352.17428, found 352.1742.

(S)-2,2′-(7-(2-((2-((2-((I-amino-3-(1H-indol-3-yl)-1-oxopropan-2-yl)amino)-2-oxoethyl)amino)-2-oxoethyl)amino)-2-oxoethyl)-1,4,7-triazonane-1,4-diyl)diacetic acid, (3). Compound 3 (150 mg, 0.093 mmol) was synthesized using the general coupling strategy outlined in Section II(a) and FIG. 13 from compound 1. The product was isolated as a colorless solid following deprotection and purification using Method A and characterized using mass spectrometry and HPLC chromatography. Yield: (11.9 mg, 0.020 mmol, 21%). Rₜ (Method B): 6.39 min. ¹H NMR (400 MHz, D₂O): δ 7.68 (d, 1H), 7.51 (d, 1H), 7.2 (m, 2H), 7.18 (t, 1H), 4.69 (t, 1H), 3.96-3.63 (m, 11H), 3.37-3.13 (m, 10H), 2.97 (s, 4H). ¹³C NMR (100 MHz, D₂O): δ 176.24 (C^{21,19}), 172.43 (C¹⁰), 171.58 (C¹²), 171.22 (C^{14,16}), 136.09 (C¹), 126.87 (C⁶), 124.22 (C⁷), 121.99 (C³), 119.36 (C⁴), 118.35 (C⁵), 111.87 (C²), 109.08 (C⁸), 58.46 (C¹¹), 56.13 (C^{18,20}), 53.89 (C¹¹), 50.51 (C^{cycl.}), 49.41 (C^{cycl.}), 48.87 (C^{cycl.}), 42.28 (C¹³), 42.20 (C¹⁵), 26.90 (C⁹). HR-ESI-MS [M+H]⁺ calc. for C₂₇H₃₈N₈O₈ 602.281262, found 603.2888.

2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7-triazonane-1,4-diyl)diacetic acid, (4). Compound 4 (150 mg, 0.093 mmol) was synthesized using the general coupling strategy outlined in Section II(a) and FIG. 13 from compound 2. The product was isolated as a colorless solid following deprotection and purification using Method A and characterized using mass spectrometry and HPLC chromatography. Yield: (11.5 mg, 0.018 mmol, 20%). Rₜ (Method B): 6.30 min. ¹H NMR (400 MHz, D₂O): δ 7.69 (d, 1H), 7.51 (d, 1H), 7.2 (m, 2H), 7.18 (t, 1H), 4.68 (t, 1H), 4.44 (t, 1H), 3.97-3.80 (m, 8H), 3.69 (s, 2H), 3.34-3.14 (m, 10H), 3.07-2.96 (m, 4H). ¹³C NMR (100 MHz, D₂O): δ 176.15 (C²⁰), 172.51 (C²²), 172.25 (C¹⁰), 172.12 (C¹⁴), 171.13 (C^{12,17}), 136.07 (C¹), 126.88 (C⁶), 124.14 (C⁷), 121.99 (C³), 119.36 (C⁴), 118.38 (C⁵), 111.86 (C²), 109.16 (C⁸), 60.69 (C¹⁶), 57.96 (C¹⁸), 56.47 (C¹⁵), 55.79 (C^{19,21}), 53.95 (C¹¹), 50.57 (C^{cycl.}), 49.60 (C^{cycl.}), 49.10 (C^{cycl.}), 42.49 (C¹³), 27.01 (C⁹). HR-ESI-MS [M+H]⁺ calc. for C₂₈H₄₀N₈O₉ 632.291827, found 633.2990.

2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl-1, 2, 3-¹³C₃)amino-¹⁵N)-2-oxoethyl)-1,4,7-triazonane-1,4-diyl)diacetic acid, (4*). Compound 4* (100 mg, 0.062 mmol) was synthesized using the general coupling strategy outlined in Section II(a) and FIG. 13 from compound 2*. The product was isolated as a white solid following deprotection and purification using Method A and characterized using mass spectrometry and HPLC chromatography. Yield: (5.1 mg, 0.008 mmol, 13%). Rₜ (Method B): 6.33 min. HR-ESI-MS [M+H]⁺ calc. for [¹³C]₃[¹⁵N]₁C₂₅H₄₁N₇O₉ 637.30675, found 637.3066.

2′,2″-(10-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7,10-tetraazacyclododecane-1,4,7-triyl)triacetic acid, (5). Compound 5 (150 mg, 0.093 mmol) was synthesized using the general coupling strategy outlined in Section II(a) and FIG. 13 from compound 2. The product was isolated as a colorless solid following deprotection and purification using Method A and characterized using mass spectrometry and HPLC chromatography. Yield: (20.2 mg, 0.028 mmol, 30%). Rₜ (Method B): 5.95 min. ¹H NMR (400 MHz, D₂O): δ 7.70 (d, 1H), 7.52 (d, 1H), 7.26 (m, 2H), 7.20 (t, 1H), 4.68 (t, 1H), 4.36 (s, 1H), 3.92 (s, 3H), 3.81 (s, 4H), 3.38-3.17 (m). ¹³C NMR (100 MHz, D₂O): δ 176.28 (C^{12,17,20,22,24}), 171.21 (C^{1,14}), 136.13 (C¹), 126.84 (C⁶), 124.34 (C⁷), 122.01 (C³), 119.34 (C⁴), 118.41 (C⁵), 111.91 (C²), 109.16 (C⁸), 60.74 (C^{11,16}), 55.63 (C^{19,21,23}) 53.95 (C^{15,18),} 42.36 (C¹³), 27.02 (C⁹). HR-ESI-MS [M+H]⁺ calc. for C₃₂H₄₇N₉O₁₁ 733.339506, found 734.3471.

(((1S)-1-carboxy-5-(2-((1r,4S)-4-((5-carboxypentanamido)methyl)cyclohexane-1-carboxamido)-3-(naphthalen-2-yl)propanamido)pentyl)carbamoyl)-L-glutamic acid, (6). Compound 6 (100 mg, 0.08 mmol) was synthesized using the general coupling strategy outlined in Section II(b) and FIG. 14 from compound WA-Glu-urea-Lys-Nap-trans. The product was isolated as a white solid following deprotection and purification using Method A and characterized using mass spectrometry and HPLC chromatography. Yield: (20.1 mg, 0.026 mmol, 32%). Rₜ (Method B): 8.28 min. HR-ESI-MS [M+H]⁺ calc. for C₃₂H₄₇N₉O₁₁ 784.37690, found 784.3762.

(((1S)-1-carboxy-5-(2-((1r,4S)-4-((6-((2,5-dioxopyrrolidin-1-yl)oxy)-6 oxohexanamido)methyl)cyclohexane-1-carboxamido)-3-(naphthalen-2-yl)propanamido)pentyl)carbamoyl)-L-glutamic acid, (7). Compound 7 (100 mg, 0.08 mmol) was synthesized using the general coupling strategy outlined in Section II(b) and FIG. 14 from compound 6. The product was isolated as a white solid following deprotection and purification using Method A and characterized using mass spectrometry and HPLC chromatography. Yield: (25.1 mg, 0.029 mmol, 36%). Rₜ (Method B): 8.85 min. HR-ESI-MS [M+H]⁺ calc. for C₃₂H₄₇N₉O₁ 881.39328, found 881.3919.

(((1S)-1-carboxy-5-(3-(naphthalen-2-yl)-2-((1r,4S)-4-((6-oxo-6-((4-((1,4,7-tris(carboxymethyl)-1,4,7-triazonan-2-yl)methyl)phenyl)amino)hexanamido)methyl)cyclohexane-1-carboxamido)propanamido)pentyl)carbamoyl)-L-glutamic acid, (8). Compound 8 (50 mg, 0.04 mmol) was synthesized using the general coupling strategy outlined in Section II(b) and FIG. 14 from compound 7. The product was isolated as a white solid following deprotection and purification using Method A and characterized using mass spectrometry and HPLC chromatography. Yield: (5.0 mg, 0.004 mmol, 11%). Rₜ (Method B): 8.25 min. HR-ESI-MS [M+H]⁺ calc. for C₃₂H₄₇N₉O₁₁ 1174.56722, found 1174.5657.

### III. Synthesis and Characterization of Coordination Complexes

### a) 3.1 General Complexation Protocol

A 5 mg aliquot of the peptide 3, 4, or 5 was dissolved in 500 μL of room-temperature distilled water. Two equivalents of the ^{nat}Ga³⁺, ^{nat}Cu³⁺, or ^{nat}Sc³⁺ salt were added to the solution, which was left to react. Complexation of the NOTA derivatives (3 and 4) occurred at room temperature for 1 h, while complexation of the DOTA derivative (5) took place at 80° C. for 3 h. Full complexation was confirmed by LCMS. The complex was then purified using a Sep-Pak Plus C18 short cartridge. The concentration was determined using ICP-OES, and the complexes were further characterized via analytical HPLC and NMR.

### b) HRMS, HR-ESI-MS and HPLC Data for Coordination Complexes

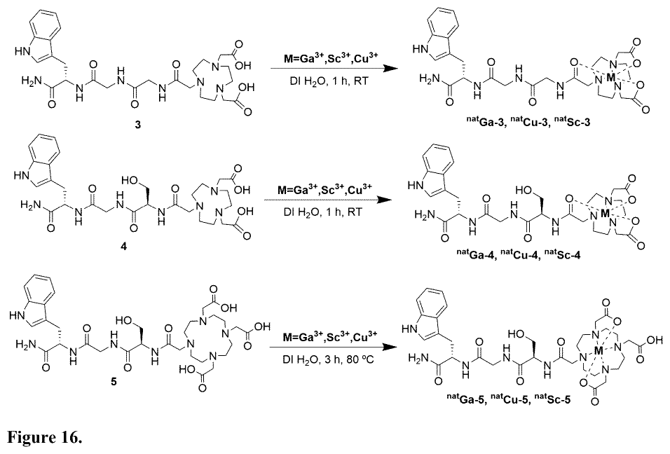

^{nat}Gallium (S)-2,2′-(7-(2-((2-((2-((1-amino-3-(1H-indol-3-yl)-1-oxopropan-2-yl)amino)-2-oxoethyl)amino)-2-oxoethyl)amino)-2-oxoethyl)-1,4,7-triazonane-1,4-diyl)diacetate, (^{nat}Ga-3). Complex ^{nat}Ga-3 (3.0 mg, 0.004 mmol, 56%) was synthesized using the general coupling strategy outlined in Section III(a) and FIG. 16 from compound 3 (5.0 mg, 0.008 mmol) and Ga(NO₃)₃·3H₂O (4.25 mg, 0.017 mmol). The product was isolated as a pale solid following complexation and purification using a Sep-Pak Plus C18 short cartridge and characterized using mass spectrometry and HPLC chromatography. Yield: (3.0 mg, 0.004 mmol, 56%). Rₜ (Method B): 5.93 min. ¹H NMR (400 MHz, D₂O): δ 7.74 (d, 1H), 7.53 (d, 1H), 7.28 (m, 2H), 7.20 (t, 1H), 4.67 (t, 1H), 4.3 (s, 2H), 4.21 (s, 2H), 3.88-3.74 (m, 4H), 3.55-3.02 (m, 17H). ¹³C NMR (100 MHz, D₂O): δ 176.35 (C²¹), 174.53 (C¹⁹), 174.42 (C¹⁰), 174.28 (C¹²), 170.65 (C¹⁴), 168.76 (C¹⁶) 136.06 (C¹), 126.91 (C⁶), 124.57 (C⁷), 121.98 (C³), 119.41 (C⁴), 118.41 (C⁵), 111.88 (C²), 109.14 (C¹), 61.83 (C¹⁸), 61.63 (C²⁰), 59.69 (C¹⁷), 54.12 (C^{cycl.}), 53.37 (C^{cycl.}), 53.29 (C^{cycl.}), 53.19 (C^{cycl.}), 53.01 (C^{cycl.}), 52.94 (C^{cycl.}), 52.79 (C^{cycl.}), 43.69 (C¹¹), 42.23 (C^{13,15}), 26.94 (C⁹). HR-ESI-MS [M+H]⁺ calc. for C₂₇H₃₆GaN₈O₈ 669.191193, found 669.1899.

^{nat}Copper (S)-2,2′-(7-(2-((2-((2-((1-amino-3-(1H-indol-3-yl)-1-oxopropan-2-yl)amino)-2-oxoethyl)amino)-2-oxoethyl)amino)-2-oxoethyl)-1,4,7-triazonane-1,4-diyl)diacetate, (^{nat}Cu-3). Compound ^{nat}Cu-3 (1.5 mg, 0.0023 mmol, 54%) was synthesized using the general coupling strategy outlined in Section III(a) and FIG. 16 from compound 3 (2.5 mg, 0.0042 mmol) and CuSO₄·5H₂O (2.07 mg, 0.0083 mmol). The product was isolated as a pale blue solid following complexation and purification using Sep-Pak Plus C18 short cartridge and characterized using mass spectrometry and HPLC chromatography. Yield: (1.5 mg, 0.0023 mmol, 54%). Rₜ (Method B): 6.90 min. HR-ESI-MS [M+H]⁺ calc. for C₂₇H₃₆CuN₈O₈ 663.195211, found 664.2020.

^{nat}Scandium (S)-2,2′-(7-(2-((2-((2-((1-amino-3-(1H-indol-3-yl)-1-oxopropan-2-yl)amino)-2-oxoethyl)amino)-2-oxoethyl)amino)-2-oxoethyl)-1,4,7-triazonane-1,4-diyl)diacetate, (^{nat}Sc-3). Compound ^{nat}Sc-3 (1.7 mg, 0.0026 mmol, 43%) was synthesized using the general coupling strategy outlined in Section III(a) and FIG. 16 from compound 4 (3.7 mg, 0.0061 mmol) and ScCl₃·6H₂O (3.19 mg, 0.012 mmol). The product was isolated as a pale white solid following complexation and purification using a Sep-Pak Plus C18 short cartridge and characterized using mass spectrometry and HPLC chromatography. Yield: (1.7 mg, 0.0026 mmol, 43%). Rₜ (Method B): 6.32 min. HR-ESI-MS [M+H]⁺ calc. for C₂₇H₃₆N₈O₈Sc 645.221526, found 645.2205.

^{nat}Gallium 2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7-triazonane-1,4-diyl)diacetate, (^{nat}Ga-4). Complex ^{nat}Ga-4 (6.2 mg, 0.009 mmol, 55%) was synthesized using the general coupling strategy outlined in Section III(a) and FIG. 16 from compound 4 (10.0 mg, 0.016 mmol) and Ga(NO₃)₃·3H₂O (8.09 mg, 0.032 mmol). The product was isolated as a colorless solid following complexation and purification using a Sep-Pak Plus C18 short cartridge and characterized by mass spectrometry and HPLC chromatography. Yield: (6.2 mg, 0.009 mmol, 55%). Rₜ (Method B): 5.97 min. ¹H NMR (400 MHz, D₂O): δ 7.71 (d, 1H), 7.53 (d, 1H), 7.28-7.29 (m, 2H), 7.19 (t, 1H), 4.71 (m, 2H), 4.38-4.26 (m, 2H), 3.97-3.81 (m, 9H), 3.76-3.16 (m, 13H), 2.98 (m, 1H). ¹³C NMR (100 MHz, D₂O): δ 176.32 (C¹⁰), 174.49 (C²²), 174.34 (C²⁰), 170.46 (C¹⁴), 169.51 (C¹²), 168.32 (C¹⁷), 136.11 (C¹), 126.92 (C⁶), 124.46 (C⁷), 121.95 (C³), 119.35 (C⁴), 118.38 (C⁵), 111.88 (C²), 108.99 (C⁸), 61.86 (C¹⁶), 61.83 (C¹¹), 60.51 (C¹⁵), 59.80 (C¹⁹), 57.58 (C²¹), 54.50 (C^{cycl.}), 54.05 (C^{cycl.}), 53.42 (C^{cycl.}), 53.33 (C^{cycl.}), 53.24 (C^{cycl.}), 53.09 (C^{cycl.}), 52.97 (C^{cycl.}), 42.34 (C¹⁸), 38.70 (C¹³), 27.04 (C⁹). HR-ESI-MS [M+H]⁺ calc. for C₂₈H₃₈GaN₈O₉ 699.201757, found 699.2015.

^{nat}Gallium 2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl-1,2,3-¹³CG)amino-¹⁵N)-2-oxoethyl)-1,4,7-triazonane-1,4-diyl)diacetate, (^{nat}Ga-4*). Compound ^{nat}Ga-4* (2.1 mg, 0.003 mmol, 47%) was synthesized using the general coupling strategy outlined in Section III(a) and FIG. 16 from compound 4* (4.0 mg, 0.0063 mmol) and Ga(NO₃)₃·3H₂O (3.21 mg, 0.013 mmol). The product was isolated as a pale brown solid following complexation and purification using a Sep-Pak Plus C18 short cartridge and characterized by mass spectrometry and HPLC chromatography. Yield: (2.1 mg, 0.003 mmol, 47%). Rₜ (Method B): 5.97 min. HR-ESI-MS [M]⁺ calc. for C₂₅[¹³C]₃H₃₈GaN₇[¹⁵N]O₉ 703.20885, found 703.2076. ^{nat}Copper 2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7-triazonane-1,4-diyl)diacetate, (^{nat}Cu-4). Compound ^{nat}Cu-4 (1.1 mg, 0.0016 mmol, 36%) was synthesized using the general coupling strategy outlined in Section III(a) and FIG. 16 from compound 4 (2.8 mg, 0.0044 mmol) and CuSO₄·5H₂O (2.21 mg, 0.0089 mmol). The product was isolated as a pale blue solid following complexation and purification using a Sep-Pak Plus C18 short cartridge and characterized using mass spectrometry and HPLC chromatography. Yield: (1.1 mg, 0.0016 mmol, 36%). Rₜ (Method B): 6.25 min. HR-ESI-MS [M+H]⁺ calc. for C₂₈H₃₈CuN₈O₉ 693.205776, found 694.2133.

^{nat}Scandium 2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7-triazonane-1,4-diyl)diacetate, (^{nat}Sc-4). Compound ^{nat}Sc-4 (1.5 mg, 0.0022 mmol, 39%) was synthesized using the general coupling strategy outlined in Section III(a) and FIG. 16 from compound 4 (3.6 mg, 0.0057 mmol) and ScCl₃·6H₂O (2.95 mg, 0.011 mmol). The product was isolated as a pale solid following complexation and purification using a Sep-Pak Plus C18 short cartridge and characterized using mass spectrometry and HPLC chromatography. Yield: (1.5 mg, 0.0022 mmol, 39%). Rₜ (Method B): 6.27 min. HR-ESI-MS [M+H]⁺ calc. for C₂₈H₃₈N₈O₉Sc 675.232091, found 675.2307.

^{nat}Gallium 2,2′,2″-(10-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7,10-tetraazacyclododecane-1,4,7-triyl)triacetate, (^{nat}Ga-5). Complex ^{nat}Ga-5 (4.8 mg, 0.006 mmol, 63%) was synthesized using the general coupling strategy outlined in Section III(a) and FIG. 16 from compound 5 (7.0 mg, 0.0095 mmol) and Ga(NO₃)₃-·3H₂O (4.88 mg, 0.019 mmol). The product was isolated as a colorless solid following complexation and purification using a Sep-Pak Plus C18 short cartridge and characterized using mass spectrometry and HPLC chromatography. Yield: (4.8 mg, 0.006 mmol, 63%). Rₜ (Method B): 5.90 min. ¹H NMR (400 MHz, D₂O): δ 7.68 (d, 1H), 7.53 (d, 1H), 7.30-7.24 (m, 2H), 7.20 (t, 1H), 4.70 (t, 1H), 4.40 (t, 1H), 4.00-3.71 (m, 15H), 3.61-3.06 (m, 16H). ¹³C NMR (100 MHz, D₂O): δ 176.10 (C¹⁰), 173.38 (C²⁰), 173.31 (C²⁴), 172.29 (C²²), 171.38 (C¹⁴), 171.09 (C¹²), 168.80 (C¹¹), 136.02 (C¹), 126.85 (C⁶), 124.08 (C⁷), 122.01 (C³), 119.41 (C⁴), 118.39 (C⁵), 111.91 (C²), 109.24 (C⁸), 61.48 (C¹⁶), 61.25 (C¹¹), 60.72 (C¹⁵), 59.29 (C¹³), 57.29 (C^{cycl.}), 57.11 (C^{cycl.}), 55.74 (C^{cycl.}), 54.79 (C^{cycl.}), 54.42 (C^{cycl.}), 54.35 (C¹⁹), 53.90 (C²¹), 42.48 (C¹⁸), 27.03 (C⁹). HR-ESI-MS [M+H]⁺ calc. For C₃₂H₄₇GaN₉O₁₁ 802.265087, found 800.2486. ^{nat}Copper 2,2′,2-(10-(2-(((R)-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7,10-tetraazacyclododecane-1,4,7-triyl)triacetate, (^{nat}Cu-5). Compound ^{nat}Cu-5 (1.3 mg, 0.0016 mmol, 40%) was synthesized using the general coupling strategy outlined in Section III(a) and FIG. 16 from compound 5 (3.0 mg, 0.0041 mmol) and CuSO₄·5H₂O (2.04 mg, 0.0082 mmol). The product was isolated as a pale blue solid following complexation and purification using a Sep-Pak Plus C18 short cartridge and characterized using mass spectrometry and HPLC chromatography. Yield: (1.3 mg, 0.0016 mmol, 40%). Rₜ (Method B): 6.12 min. HR-ESI-MS [M+H]⁺ calc. for C₃₂H₄₇CuN₉O₁₁ 796.269105, found 795.2597.

^{nat}Scandium 2,2′,2″-(10-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7,10-tetraazacyclododecane-1,4,7-triyl)triacetate, (^{nat}Sc-5). Compound ^{nat}Sc-5 (1.8 mg, 0.002 mmol, 42%) was synthesized using the general coupling strategy outlined in Section III(a) and FIG. 16 from compound 5 (4.0 mg, 0.0055 mmol) and ScCl₃·6H₂O (2.83 mg, 0.011 mmol). The product was isolated as a pale solid following complexation and purification using a Sep-Pak Plus C18 short cartridge and characterized using mass spectrometry and HPLC chromatography. Yield: (1.8 mg, 0.002 mmol, 42%). Rₜ (Method B): 5.75 min. HR-ESI-MS [M+H]⁺ calc. for C₃₂H₄₇N₉O₁₁Sc 778.295419, found 776.2796.

^{nat}Gallium (((1S)-1-carboxy-5-(3-(naphthalen-2-yl)-2-((1r,4S)-4-((6-oxo-6-((4-((1,4,7-tris(carboxymethyl)-1,4,7-triazonan-2-yl)methyl)phenyl)amino)hexanamido)methyl)cyclohexane-1-carboxamido)propanamido)pentyl)carbamoyl)-L-glutamic acid triacetate, (^{nat}Ga-8). Compound ^{nat}Ga-8 (1.5 mg, 0.0012 mmol, 46%) was synthesized using the general coupling strategy outlined in Section III(a) and FIG. 14 from compound 8 (3.0 mg, 0.0026 mmol) and Ga(NO₃)₃·3H₂O (1.33 mg, 0.0052 mmol,). The product was isolated as a white solid following complexation and purification using a Sep-Pak Plus C18 short cartridge and characterized using mass spectrometry and HPLC chromatography. Yield: (1.5 mg, 0.0012 mmol, 46%). Rₜ (Method B): 7.93 min. HR-ESI-MS [M+H]⁺ calc. for C₅₈H₇₇GaN₉O₁₇ 1240.46932, found 1240.4690.

### IV. Synthesis and Characterization of Radiochemical Complexes

### a) General Radiolabeling Protocol

### 1. Radiolabeling with ⁶⁷Ga

⁶⁷Ga-citrate was purchased from Jubilant Radiopharma at an average specific activity of 205.0 MBq/mL. The ⁶⁷Ga-citrate solution was first converted to ⁶⁷GaCl₃ using an established solid-phase extraction protocol (Pandy, 2021). The resulting average specific activity of the obtained ⁶⁷Ga-chloride solution used for solution radio-labelling was 80.5 MBq/mL. For radio-labeling of NOTA-based model peptides or compound 10, a 20 μL aliquot containing 4.5 MBq of ⁶⁷GaCl₃ was added to the conjugate (10 nmol, 300 μL) in 10 mM of sodium acetate (NaOAc) buffer. The pH of the solution was adjusted to 5.0. Radiolabeling was completed after 20 minutes at room temperature. The radiolabeled conjugates were characterized with radio-HPLC.

### 2. Radiolabeling with ⁶⁸Ga

⁶⁸Ga-chloride was obtained from Dr. Jacob Houghton, Department of Radiology, Stony Brook University, Stony Brook, New York, at an average specific activity of 370.0 MBq/mL. For radio-labeling of NOTA-Bn-Ada-PSMA, a 1.0 mL aliquot containing 38.8 MBq of ⁶⁸GaCl₃ was added to the resin-bound conjugate (10 nmol, 300 L) in 1.0 M of sodium acetate (NaOAc) buffer. The pH of the solution was adjusted to 5.0. Radiolabeling was completed after 20 minutes at room temperature. The radiolabeled conjugate was photocleaved for 10 min and characterized with radio-HPLC.

### 3. Synthesis of ⁶⁷Ga-Radiolabeled Complexes

⁶⁷Gallium (S)-2,2′-(7-(2-((2-((2-((I-amino-3-(1H-indol-3-yl)-1-oxopropan-2-yl)amino)-2-oxoethyl)amino)-2-oxoethyl)amino)-2-oxoethyl)-1,4,7-triazonane-1,4-diyl)diacetate (⁶⁷Ga-3). Compound ⁶⁷Ga-3 was synthesized using the general radiolabeling protocol from compound 3. The product was characterized using radio-HPLC chromatography. Rₜ (Method D): 6.02 min. SA: 10.0 μCi/nmol, RCY: 99%, RCP: 99%.

⁶⁷Gallium 2,2′-(7-(2-((1-1-((2-(((S)-1-amino-3-(1H-indol-3-yl)-1-oxopropan-2-yl)amino)-2-oxoethyl)amino)-3-hydroxy-1-oxopropan-2-yl)amino)-2-oxoethyl)-1,4,7-triazonane-1,4-diyl)diacetate (⁶⁷Ga-4). Compound ⁶⁷Ga-4 was synthesized using the general radiolabeling protocol from compound 6. The product was characterized using radio-HPLC chromatography. R (Method D): 6.05 min. SA: 10.0 μCi/nmol, RCY: 99%. RCP: 99%.

⁶⁷Gallium 2,2′,2″-(10-(2-((1-1-((2-(((S)-1-amino-3-(H-indol-3-yl)-1-oxopropan-2-yl)amino)-2-oxoethyl)amino)-3-hydroxy-1-oxopropan-2-yl)amino)-2-oxoethyl)-1,4,7,10-tetraazacyclododecane-1,4,7-triyl)triacetate (⁶⁷Ga-5). Compound ⁶⁷Ga-5 was synthesized using the general radiolabeling protocol from intermediate 5. The product was characterized using radio-HPLC chromatography. Rₜ (Method D): 5.95 min. SA: 10.0 ρCi/nmol, RCY: 99%, RCP: 99%.

### 4. Synthesis of 68Ga-Radiolabeled Complexes

⁶⁸Gallium (((1S)-1-carboxy-5-(3-(naphthalen-2-yl)-2-((1r,4S)-4-((6-oxo-6-((4-((1,4,7-tris(carboxymethyl)-1,4,7-triazonan-2-yl)methyl)phenyl)amino)hexanamido)methyl)cyclohexane-1-carboxamido)propanamido)pentyl)carbamoyl)-L-glutamic acid triacetate, (⁶⁸Ga-8). Compound ⁶⁷Ga-8 was synthesized using the general radiolabeling protocol from ligand 8. The product was characterized using radio-HPLC chromatography. Rₜ (Method D): 7.93 min. SA: 10.0 μCi/nmol, RCY: 95%, RCP: 95%.

### V. VTMS and CIVP Data

Data Analysis and Detailed Discussion. The variable temperature mass spectra and corresponding speciation curves of the protonated tripeptide+NOTA (4) and the Ga-tripeptide-NOTA complex (^{nat}Ga-4) are shown in FIGS. 71-75. At the highest temperature shown in both figures, no water adduct is observed. The first hydrate of the ligand and the Ga-complex were observed at 220 K. The speciation curves (FIGS. 72 and 74) show the sequential addition of multiple water molecules at lower temperature. The gas-phase enthalpies, entropies, and free energies of hydration for the 1^{st} hydrate are reported in Table 3 below. ΔG²⁹⁸ for the hydrated Ga complex was found to be well within the range of possible ΔG²⁹⁸ values for a second sphere water (−34 kJ/mol) reported (Garand, 2012). Similar values were found for the metal-free complex, further confirming that only second sphere hydration events were observed.

TABLE 3
ΔH, ΔS and ΔG²⁹⁸ (kJ/mol) values for the binding
of the first second sphere water for the tripeptide +
NOTA and Ga-tripeptide-NOTA complexes as determined by VTMS.

| Compound | ΔH (kJ/mol) | ΔS(J/mol K) | ΔG (kJ/mol) |
|---|---|---|---|
| Tripeptide + NOTA | −55.4 +/− 0.5 | −91.3 +/− 2.2 | −28.2 +/− 0.8 |
| Ga-Tripeptide-NOTA | −48.9 +/− 2.6 | −62.9 +/− 17.5 | −30.1 +/− 2.7 |

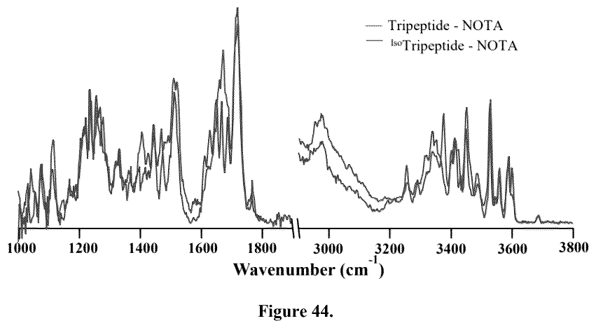

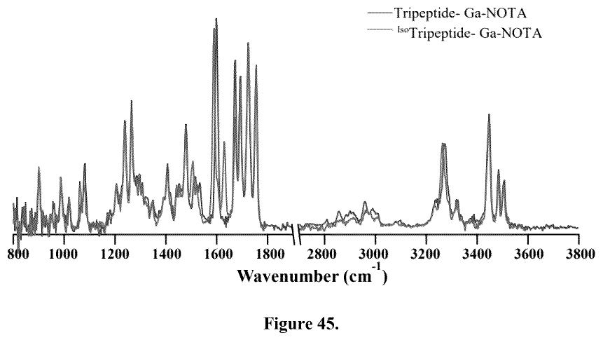

CIVP spectra of the tripeptide+NOTA ligand and its isotopologue are shown in FIG. 44 and the Ga complex, and the Ga complex and its isotopologue are shown in FIG. 45. In all cases, notable shifts are seen in the Amide A, I, and II regions as expected for isotopic substitution on serine. Focusing on the Ga-complex, a single peak isotopically shifted by 8 cm⁻¹ is observed for a strong transition near 3271 cm⁻¹, identifying this feature as the serine NH stretch undergoing a weak internal hydrogen bond commonly seen for gas phase peptides (Garand, 2012 and Meyer, 2022). The carbonyl stretching region between 1600-1800 cm⁻¹ serves as an efficient diagnostic tool in elucidating the structure of the complex. To that end, understanding the carbonyl signature in the ligand was the first logical step in the spectroscopic structural elucidation of the complex. Focusing first on the metal-free complex, ¹³C and ¹⁵N isotopic substitution of the serine in the tripeptide backbone established that the two higher energy peaks belong to the carbonyl groups in the NOTA moiety. Specifically, these transitions are derived from the OCO antisymmetric stretching modes of the C═O in NOTA similar to those found for EDTA-bound metal ions (Foreman, 2022), and are unaffected by isotopic labeling. The much broader, lower energy feature is a composite peak corresponding to the carbonyls in the tripeptide backbone. For the native isotopomer, this feature is only partially resolved, but for the isotope-exchanged species, it clearly splits into five distinguishable peaks. It was only expected six total carbonyl stretches in this complex, suggesting the coexistence of at least two isomers in the absence of Ga. Upon complexation with Ga, there were five sharp, distinct peaks in the same spectral region of the that can be separated in three types of carbonyls in the complex. The two highest energy peaks remain the carbonyls in the NOTA, having undergone slight shifts and changes in intensity due to complexation with Ga. The next two lower energy peaks can be identified as the carbonyls in the tripeptide back bone, falling in the usual range for such modes (Meyer, 2022). Indeed, upon isotopic substitution, one of these two peaks reduces in intensity, with a new peak lower in energy by 42 cm⁻¹ appearing consistent with expectations for ¹³C substitution (Garand, 2012).

This suggests that the lower energy of the two peaks is composed of two amide carbonyl stretches, fully accounting for all carbonyls in the complex. The identity of lowest-energy, the brightest peak in the spectrum, centered at 1603 cm⁻¹ is also confirmed by isotopic substitution of serine in the Ga-complex (third spectrum in FIG. 5). The 13 cm⁻¹ red shift is too small to be caused by isotopic substitution of the carbonyl carbon. However, it is consistent with substitution of the adjacent nitrogen and carbon, as motions of these atoms are coupled to motions of the carbonyl carbon and oxygen and thus isotopic substitution will indirectly increase the reduced mass of this normal mode. Thus, this peak is best assigned to the carbonyl on NOTA. The fact that it is significantly red shifted from the expected position in the amide I region confirms that it is coordinated with the metal center through the oxygen. This assignment agrees with stretching frequencies of carbonyls involved in coordination via the oxygen in complexes of divalent cations and acetylacetonate ligands (Seco, 1989). A conceivable explanation for the large red shift of the coordinated carbonyl is that coordination to the Ga³⁺ metal center weakens the double bond character of the C═O bond, moving the vibrational energy of this mode towards that of an alcohol C—O stretch typically observed near 1000-1200 cm⁻¹.

TABLE 4
Percent of each species as a function of time at pH 4.5
during 80° C. incubation of ^{nat}Ga-4. Values reported
as an average of n = 2 assays.

| Time (h) | % Complex | % Intermediate | % Peptide |
|---|---|---|---|
| 0 | 100.00 ± 0.00 | 0.00 ± 0.00 | 0.00 ± 0.00 |
| 1 | 38.54 ± 1.31 | 38.30 ± 2.07 | 23.17 ± 0.76 |
| 2 | 27.10 ± 0.42 | 35.98 ± 1.74 | 36.93 ± 2.16 |
| 3 | 20.57 ± 0.35 | 30.66 ± 1.19 | 48.77 ± 1.54 |
| 6 | 6.38 ± 1.96 | 23.71 ± 0.78 | 69.91 ± 3.74 |
| 24 | 0.00 ± 0.00 | 8.28 ± 1.68 | 91.72 ± 1.68 |

TABLE 5
Percent of each species as a function of time at pH 5.5
during 80° C. incubation of ^{nat}Ga-4. Values reported
as an average of n = 3 assays.

| Time (h) | % Complex | % Intermediate | % Peptide |
|---|---|---|---|
| 0 | 100.00 ± 0.00 | 0.00 ± 0.00 | 0.00 ± 0.00 |
| 1 | 21.80 ± 1.00 | 68.74 ± 1.75 | 9.45 ± 1.15 |
| 2 | 18.58 ± 0.36 | 63.41 ± 2.35 | 18.02 ± 2.01 |
| 3 | 15.01 ± 0.38 | 59.79 ± 1.78 | 25.20 ± 1.92 |
| 6 | 9.18 ± 0.64 | 47.71 ± 1.63 | 43.11 ± 1.00 |
| 11 | 6.35 ± 0.51 | 39.79 ± 0.88 | 53.86 ± 1.38 |
| 22 | 1.26 ± 1.11 | 24.03 ± 2.19 | 74.71 ± 1.14 |

TABLE 6
Percent of each species as a function of time at pH 6.5
during 80° C. incubation of ^{nat}Ga-4. Values reported
as an average of n = 3 assays.

| Time (h) | % Complex | % Intermediate | % Peptide |
|---|---|---|---|
| 0 | 78.96 ± 4.25 | 21.04 ± 4.25 | 0.00 ± 0.00 |
| 1 | 18.05 ± 0.37 | 73.69 ± 1.18 | 8.27 ± 1.35 |
| 2 | 16.27 ± 0.48 | 68.07 ± 1.33 | 15.66 ± 1.02 |
| 3 | 14.03 ± 0.56 | 63.85 ± 1.99 | 22.12 ± 1.46 |
| 6 | 7.99 ± 1.96 | 54.19 ± 1.44 | 37.82 ± 1.23 |
| 22 | 0.00 ± 0.00 | 26.76 ± 1.03 | 73.24 ± 1.03 |

TABLE 7
Percent of each species as a function of time at pH 7.5
during 80° C. incubation of ^{nat}Ga-4. Values reported
as an average of n = 3 assays.

| Time (h) | % Complex | % Intermediate | % Peptide |
|---|---|---|---|
| 0 | 60.78 ± 10.2 | 39.22 ± 10.2 | 0.00 ± 0.00 |
| 1 | 15.83 ± 0.55 | 79.32 ± 0.06 | 4.85 ± 0.48 |
| 2 | 14.36 ± 2.17 | 76.77 ± 1.70 | 8.87 ± 0.71 |
| 3 | 13.21 ± 2.29 | 73.46 ± 2.23 | 13.33 ± 1.11 |
| 6 | 10.85 ± 0.16 | 63.13 ± 0.39 | 26.02 ± 0.24 |
| 10 | 8.57 ± 0.46 | 55.97 ± 1.27 | 35.46 ± 1.63 |
| 23 | 4.88 ± 0.37 | 31.59 ± 0.65 | 63.53 ± 0.99 |

TABLE 8
Kinetic rates of reaction calculated for each measured pH using a first-
order kinetic fit. Values reported as an average of n = 3 assays.

| pH | N,O rearrangement rate (h⁻¹) | Ester hydrolysis rate (h⁻¹) |
|---|---|---|
| 4.5 | 0.365 ± 0.071 | 0.107 ± 0.008 |
| 5.5 | 0.172 ± 0.006 | 0.062 ± 0.008 |
| 6.5 | 0.172 ± 0.058 | 0.069 ± 0.009 |
| 7.5 | 0.0663 ± 0.006 | 0.401 ± 0.003 |

TABLE 9
Quantitation of the ⁶⁸Ga(8) product released
from TG-10 per time vs. cumulative release.

| Time | Activity released | Cumulative released |
|---|---|---|
| (min) | per time [%] | activity [%] |
| 10 | 14.2 ± 1.57 | 14.2 ± 1.57 |
| 20 | 17.9 ± 0.97 | 32.1 ± 0.60 |
| 30 | 18.9 ± 0.61 | 50.9 ± 1.21 |
| 60 | 15.4 ± 0.85 | 66.3 ± 0.36 |
| 90 | 11.5 ± 0.84 | 77.8 ± 1.20 |
| 120 | 9.2 ± 1.56 | 87.0 ± 2.76 |
| | | 88.0 |

### VI. Cleavage Assay for Cold Complexes

Cleavage of the cold model complexes was observed over a range of physiological pH conditions, including pH values of 4.5, 5.5, 6.5, and 7.5. Aqueous buffer solutions were prepared using 10 mM sodium acetate (pH 4.5 and 5.5) or 10 mM HEPES (pH 6.5 and 7.5), which were subsequently adjusted to the desired pH using NaOH and/or HCl. An aliquot of the complex was placed in an aqueous solution of the desired pH and incubated for 48 hours at 80° C., monitored regularly via LCMS.

### DISCUSSION

A traceless, efficient photochemical bond cleavage strategy could efficiently release the target radiopharmaceutical into solution following isotope capture and removal of solution impurities. This invention provides a development and optimization of Solid Phase RadioMetalchelation and Photorelease (SoPhRaMP) as a modular radiosynthesis approach of radiometal-based radiopharmaceuticals. A short, NOTA chelator-linked model tri-peptide was developed to optimize resin attachment, radiolabeling volume, activity scope and photocleavage with the radioactive isotopes ⁶⁷Ga and ⁶⁴Cu. Subsequently, two chelator-linked peptides with defined, clinically relevant peptide sequences using pre and post-synthetic resin linkage strategy were synthesized and demonstrated efficient radiochemical labeling of both under previously established conditions. Finally, it was shown that SoPhRaMP can efficiently exploit the differences in pH dependent speciation of metal ion chelation to selectively capture ⁶⁷Ga and ⁶⁴Cu isotopes in presence of a 5-orders of magnitude excess of stable Zn²⁺ and Ni²⁺ isotopes.

Firstly, model peptide NOTA-Aca-Q-W (10) has been prepared on different types of solid supports, which all are a) compatible with aqueous conditions essentials for radiolabeling, b) provide stability of resin-attached peptides during acidic deprotection step. In order to obtain such constructs, the chelating peptide with 3-amino-3-(2-nitrophenyl)propionic acid (ANP) as a photosensitive moiety (4) was synthesized on chlorotrityl resin (2-CTC) using standard solid-phase peptide synthesis (SPPS) approach. The NOTA-Aca-Q-WANP-H conjugate (4), has been cleaved in mild acidic conditions yielding in protected peptide bearing free C-terminus (5). Subsequently, it was converted selectively in NHS-activated peptide (7) and then all protecting groups have been removed (8). The purified peptide 8 has been loaded into three types of solid supports, namely tentagel, agarose-based and ion-exchange resin. The successful synthesis of model peptide 10 has been confirmed by photocleavage at 365 nm followed by MS and HPLC characterization.

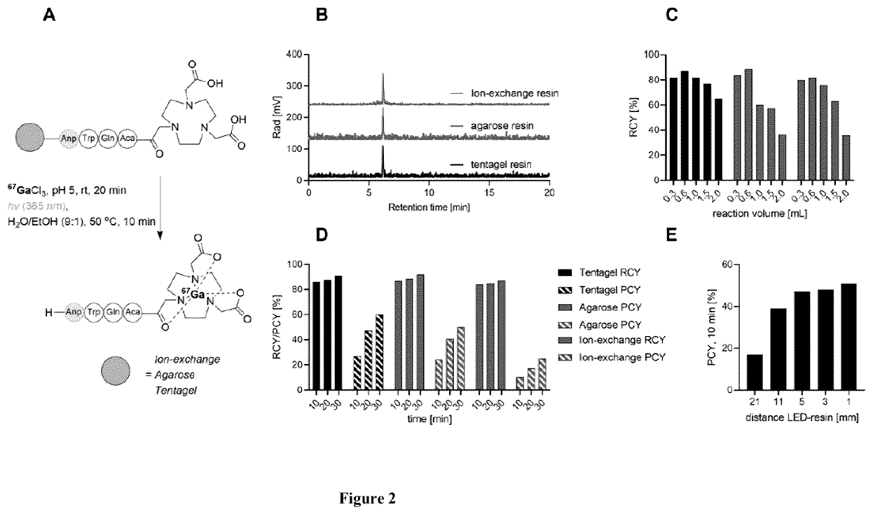

Having these compounds in hands, coordinatization with ⁶⁷Ga was tested in order to confirm the feasibility of radiolabeling on solid support followed by photocleavage and filtration in one cycle. All types of solid supports loaded with peptide have been radiolabeled directly with 50 μCi of 67Ga at room temperature for different time points FIG. 2 A. As can be seen from the FIG. 2 D, high radiochemical (RCY) yields were observed for all types of solid supports within 10 min. Further, extending the solid-phase radiolabeling time resulted in increased radiochemical yield, giving RCY over >90% within 30 min for tentagel-based and agarose-based solid support. Having defined the radiolabeling conditions, this invention moved on to the experimental evidence on photocleavage of radiolabeled peptide from solid support. To each type of solid support with radiolabeled peptide, 300 μL of mixture 10% ethanol in water was added and the photocleavage at 365 nm has been started. The 10 min photocleavage gave moderate photochemical yield (PCY) over >20%. The FIG. 2 D confirms that extending the photocleavage time increases steadily the photochemical yield, resulting in PCY over >50% within 30 min for tentagel-based and agarose-based solid support. What stands out in this figure is the decline in the photocleavage yield when the ion-exchange resin is involved. Each type of solid support resulted in excellent radiochemical purity of ⁶⁷Ga-NOTA-Aca-Q-W (FIG. 2 B). The photocleavage was also carried out at room temperature, showing a slight decrease in PCY for all the types of solid supports using the same conditions.

To evaluate the feasibility of the selective capture of radioactivity from highly diluted generator eluates, the volume-dependent solidphase radiolabeling has been examined. The radiolabeling was carried out using 0.3, 0.6, 1.0, 1.5 and 2.0 mL of radiolabeling mixture. As shown in the FIG. 2 C that even for high radiolabeling volume (≥1.0 mL), the RCY stays at the high level (≥80%) for tentagel and ≥70% for agarose resin. From these data, it was shown only agarose-based resin resulted in the lowest RCY values starting from 1.0 mL volume. Taking account of that working with resins requires the appropriate handling, namely prewashing and washing in order to swell the resin as it is routinely done in the standard SPPS approach, this invention further investigates whether or not the time-consuming washing step has any influence on radiolabeling step. The standard swelling process involves washing the resin with DCM, DMF, DMF/H₂O, H₂O and finally with the appropriate buffer dedicated to the radiolabeling. This whole step consumes 20 min and requires of using organic solvents. Two parallel radiolabeling reactions have been carried out, one with swelled, preloaded tentagel resin, the second one with dry resin. The single most striking observation to emerge from the data comparison is that there is no significant difference in radiochemical yield between swollen and dry resin.

The optimal conditions for photocleavage of radiopharmaceuticals from solid supports remain elusive. In the first step, variety of solvent systems have been used for the photocleavage step in order to determinate the solvent influence on PCY. The lowest yield has been obtained for 100% DMSO, 100% EtOH and 100% DI H₂O and the highest PCY values for 10% ethanol and 10% DMSO in water. Additionally, the putative influence of photoreactor type on photocleavage yield has been investigated. The photocleavage capacity of photoreactor equipped with single-syringe-reaction configuration was compared with the data obtained for commercially available UV Chamber Photoreactor.

When using the WPP reactor with single-syringe setup, 40% yield has been obtained within only 10 min. The UV chamber achieved only 8.5% yield after 10 min of irradiation. The maximal PCY has been obtained within 20 min when WPP reactor has been used, whereas the same value of RCY for UV Chamber Photoreactor was achieved after 1 hour. The photoreactor fitted with a 365 nm LED star decreased reaction time in comparison to a more convention UV light curing chamber.

Another significant aspect of photochemical cleavage is that this process depends not only on type of light source, but also on physical parameters as temperature and the distance between LED source and syringe vessel. Since, the photoreactor designed for SoPhRaMP is equipped with internal low-profile cooling fan system and distance-dependent holders, two sets of experiments have been designed.

Firstly, the distance-dependent photocleavage experiment without cooling fan has been carried out. Decreasing the distance between LED source and resin-containing syringe causes a gradual rise of temperature as well as the increase in PCY, starting from 25% for 21 mm to 52% for 3 mm distance. The FIG. 2 E presents the same distance-dependent photocleavage but with the cooling system switched on, which has impact on temperature as well as the PCY, giving 42% yield for 5 mm distance at 37° C.

Based on preliminary data from previous sections, it was identified that tentagel-NH₂ resin with 0.26 mmol/g loading, functionalized with Anp as a suitable solid-phase attachment compatible with the aqueous and pH-sensitive nature of radiometal coordination chemistry.

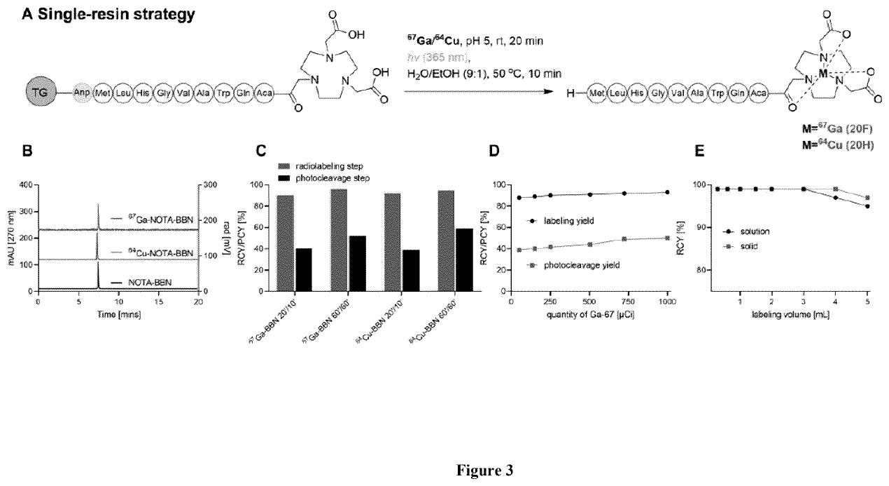

Having optimized the conditions for whole SoPhRaMP concept as shown in FIG. 2 using non-targeting tripeptide, this invention further confirms the feasibility of using two prostate targeting peptides (BBN, targeting gastrin-releasing peptide receptor and PSMA, targeting the prostate-specific membrane antigen) and two radioisotopes ⁶⁷Ga and ⁶⁴Cu. Firstly, the bombesin peptide has been synthesized directly on tentagel resin as the most compatible resin for radiolabeling and photocleavage using single resin strategy (FIG. 3 A). The direct solid-phase radiolabeling of the BBN with ⁶⁷Ga resulted in high radiochemical yield (>85%) at pH 5.0 and incubation at RT, without the need for any organic solvent additives. The similar high radiochemical yield (>88%) has been obtained for direct 20 min radiolabelling with ⁶⁴Cu using the same conditions. The followed photocleaved for 10 min produced the desired products at high radiochemical purity (RCP) (FIG. 3 B). With data confirming the solid-phase radiolabeling and photocleavage resulting in the formation of the desired species, this invention optimizes the whole SoPhRaMP cycle in order to provide the concise and efficient protocol. The radiolabeling as well as photocleavage have been carried out in 2 sets of experiments. The first set (standard protocol) involves the 60 min of radiolabeling and 60 min of photocleavage giving 2 hours of total cycle, whereas the second set (fast protocol) relies on 20 min of radiolabeling and 10 min of photocleavage (30 min total). From FIG. 3 C, it can be seen that 60 min radiolabeling resulted in RCY >88% for 64Cu-BBN and >90% for 64Cu-BBN. The 20 min low-temperature radiolabeling gave >85% RCY for 64Cu-BBN and >84% for 64Cu-BBN. Similarly, the photocleavage was performed using two protocols: 60 min and 10 min. The 60 min protocol showed the labeled constructs with PCY of ≥60% for ⁶⁷Ga-BBN and >51% for ⁶⁴Cu-BBN while 10 min protocol resulted in ≥40% for ⁶⁷Ga-BBN and >41% for ⁶⁴Cu-BBN (FIG. 3 C). Taken together the data from standard vs. fast protocol, it was recommended that the fast protocol for the SoPhRaMP cycle to provide ultrafast, robust and selective synthesis of radiopharmaceuticals. Finally, the ⁶⁷Ga-NOTA-BBN and ⁶⁴Cu-NOTA-BBN conjugates were prepared using optimized fast SPRS protocol with a variety of specific activity starting from 5 μCi/nmol to high specific activity 0.1 mCi/nmol (FIG. 3 D).

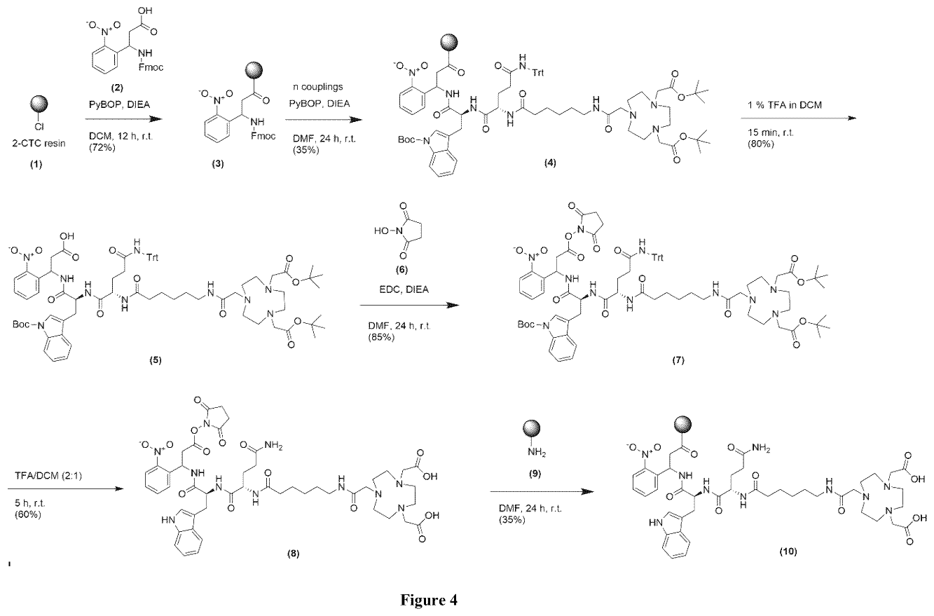

To ultimately proof the versatility of SoPhRaMP, the second NOTA-containing prostate-targeting peptide has been prepared using terminal-resin-loading strategy (FIG. 4 A), which is not restricted to classical C-terminus coupling. The NOTA-PSMA has firstly been synthesized on CTC resin, activated with NHS, cleaved, purified and eventually coupled to tentagel resin.

In summary, this invention showed for the first time the full formation of radiopharmaceuticals using solid phase approach in one-pot procedure. In the course of these investigations the successful radiolabeling of two prostate targeting peptides, NOTA-Aca-BBN and NOTA-PSMA, on resin with ⁶⁷Ga and ⁶⁴Cu and the followed photocleavage was demonstrated. Additionally, this invention optimized a convenient fast protocol for radiolabeling and photocleavage. This system uses the resin beads and anchored photo-reactive amino acid to provide the selective photochemical cargo release. Taken together, the creative and robust solution for the efficient capture and release of radioactive isotopes in order to improve the synthesis of radiopharmaceuticals, recycle spent nuclear fuel and clean up contaminated environmental samples has been provided.

### REFERENCE

Abd-Elgaliel, W. R.; Gallazzi, F.; Lever, S. Z. J. J. o. p. s. a. o. p. o. t. E. P. S., Total solid-phase synthesis of bombesin analogs with different functional groups at the C-terminus. 2007, 13 (7), 487-492.
Foreman, M. M.; Weber, J. M., Ion Binding Site Structure and the Role of Water in Alkaline Earth EDTA Complexes. J Phys. Chem. Lett. 2022, 13 (36), 8558-8563.
Garand, E.; Kamrath, M. Z.; Jordan, P. A.; Wolk, A. B.; Leavitt, C. M.; McCoy, A. B.; Miller, S. J.; Johnson, M. A., Determination of Noncovalent Docking by Infrared Spectroscopy of Cold Gas-Phase Complexes. Science 2012, 335 (6069), 694-698.
Hackenberger, C. P. J. O.; Chemistry, B., The reduction of oxidized methionine residues in peptide thioesters with NH 4 I-Me 2 S. 2006, 4 (11), 2291-2295.
Lampkin, P. P.; Thompson, B. J.; Gellman, S. H. J. O. L., Versatile Open-Source Photoreactor Architecture for Photocatalysis Across the Visible Spectrum. 2021, 23 (13), 5277-5281.
Lee, L. L.; Buckton, L. K.; McAlpine, S. R. J. P. S., Converting polar cyclic peptides into membrane permeable molecules using N-methylation. 2018, 110 (3), e24063.
Meyer, K. A.; Nickson, K. A.; Garand, E., The impact of the electric field of metal ions on the vibrations and internal hydrogen bond strength in alkali metal ion di- and triglycine complexes. J. Chem. Phys. 2022, 157 (17), 174301.
Pandey, A.; Smilowicz, D.; Boros, E., Galbofloxacin: a xenometal-antibiotic with potent in vitro and in vivo efficacy against S. aureus. Chem. Sci. 2021, 12 (43), 14546-14556.
Racow, E. E.; Kreinbihl, J. J.; Cosby, A. G.; Yang, Y.; Pandey, A.; Boros, E.; Johnson, C. J., General approach to direct measurement of the hydration state of coordination complexes in the gas phase: Variable temperature mass spectrometry. J. Am. Soc. Mass Spectrom. 2019, 141 (37), 14650-14660.
Seco, M., Acetylacetone: a versatile ligand. J Chem Educ. 1989, 66 (9), 779.
Śmilowicz, D.; Schlyer, D.; Boros, E.; Meimetis, L., Evaluation of a Radio-IMmunoStimulant (RIMS) in a Syngeneic Model of Murine Prostate Cancer and ImmunoPET Analysis of T-cell Distribution. Mol. Pharm. 2022, 19 (9), 3217-3227
Vaughn, B. A.; Loveless, C. S.; Cingoranelli, S. J.; Schlyer, D.; Lapi, S. E.; Boros, E. J. M. p., Evaluation of 177Lu and 47Sc Picaga-Linked, Prostate-Specific Membrane Antigen-Targeting Constructs for Their Radiotherapeutic Efficacy and Dosimetry. 2021, 18 (12), 4511-4519.

1. A process for preparing a compound of formula I or a compound of formula II, or a pharmaceutically acceptable salt of the compound of formula I or the compound of formula II having the structure: 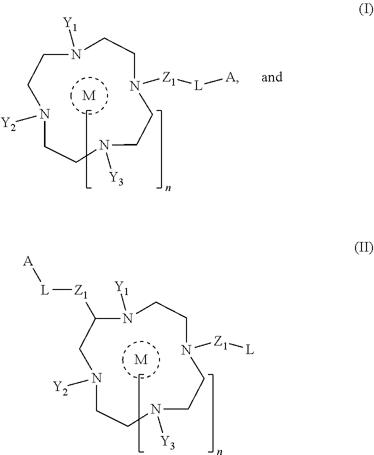
wherein
A is a targeting moiety, or an activating group or absent;
L is a peptide linker;
M is a metal that is either absent or present;
n is 0 or 1;
Y₁, Y₂, Y₃ are each, independently, —H, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N (alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ and alkylheteroaryl-P(O)(OH)₂, and
wherein each occurrence of R₄ is independently, —H, alkyl, alkenyl, alkynyl, alkyl-aryl, alkyl-heteroaryl, aryl, heteroaryl, alkyl-CF₃ or —Si(alkyl)₃;
Z₁ is 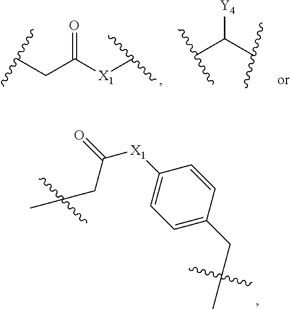
wherein X₁ is NH, O or S, and
Y₄ is —CO₂H, —CO₂R₅, aryl-CO₂H, heteroaryl-CO₂H, aryl-CO₂R₅ or heteroaryl-CO₂R₅, and
wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
wherein if the process is for producing compound of formula I when M is not present, then the process comprises:
(a) preparing a compound of formula I-A, 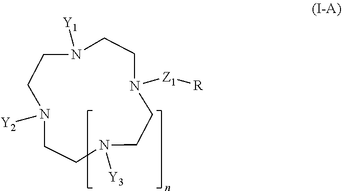
and
(b) cleaving the compound of formula I-A or the compound of II-A off a solid support surface;
wherein if the process is for producing compound of formula I when M is present, then the process comprises:
(a) preparing a compound of formula I-A, 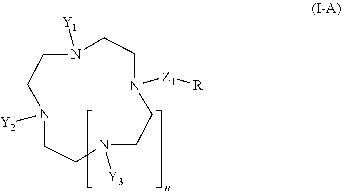
(b) complexing a metal ion with the compound of formula I-A, and
(c) cleaving the compound of formula I-A off a solid support surface;
wherein if the process is for producing compound of formula II when M is not present, then the process comprises:
(a) preparing a compound of formula II-A, 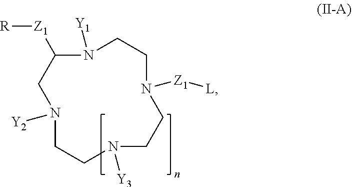
and
(b) cleaving the compound of formula II-A off a solid support surface; or
wherein if the process is for producing compound of formula II when M is present, then the process comprises:
(a) preparing a compound of formula II-A, 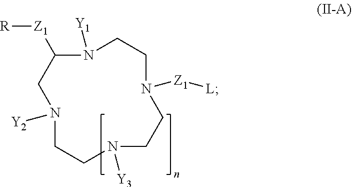
(b) complexing a metal ion with the compound of formula I-A, and
(c) cleaving the compound of formula II-A off a solid support surface;
wherein R is

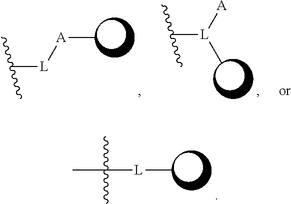

2. The process of claim 1,
[A]
wherein the process further comprises the following steps for producing the compound of formula I-A:
a) coupling a compound containing groups A and L to a solid support surface;
b) coupling the compound in step (a) with a compound of formula (III) using a coupling reagent in presence of base and a solvent: 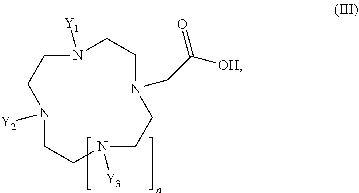
wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃;
c) reacting the compound obtained in step (b) with the following compound using a coupling reagent in presence of base and a solvent: 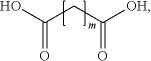
where in m is 1-20; and
d) adding an active ester synthesis reagent;
[B]
wherein the process further comprises the following steps for producing the compound of formula I-A:
a) coupling an acid containing a fluorenylmethoxycarbonyl protecting group (Fmoc) using a coupling reagent in the presence of a base to a solid support surface in a solvent;
b) deprotecting the Fmoc group with a deprotection reagent in the presence of a solvent and coupling groups A and L to the acid using a coupling reagent in the presence of a base;
c) coupling an acid containing a Fmoc protection group using a coupling reagent in the presence of a base and a solvent to the compound obtained in step (b); and
d) deprotecting the Fmoc group with a deprotection reagent and coupling the compound obtained in step (c) with the compound of formula (III) in a solvent: 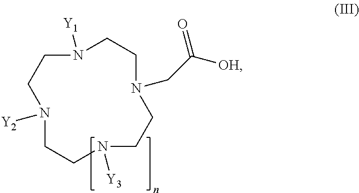
wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃;
[C]
wherein the process further comprises the following steps for producing the compound of formula II-A:
a) coupling an active ester synthesis reagent to the following compound in the presence of a coupling reagent and a base 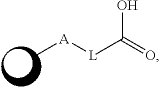
and
b) coupling the compound obtained in step (a) with the following compound in a base and a solvent 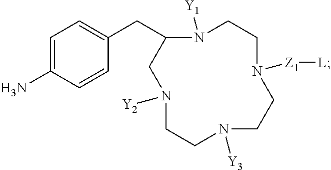
wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃;
[D]
wherein the process further comprises the following steps for producing the compound of formula II-A:
a) reacting a compound of formula IV 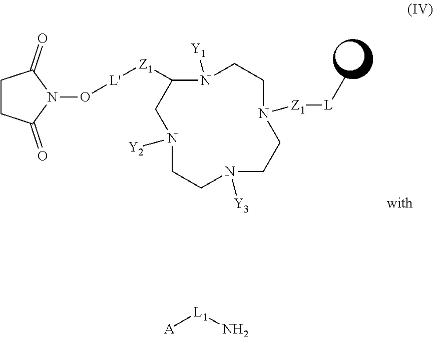
wherein L′ and L₁ are peptide linkers
[E]
a) for producing the compound of formula I when M is not present, wherein
i) step (a) and step (b) are conducted in one pot;
ii) the compound of formula I-A is photo cleaved at a wavelength about 300-400 nm; preferably at 365 nm; and/or
iii) the compound of formula I-A is chemically cleaved; preferably the compound of formula I-A is chemically cleaved by a mixture of trifluoroacetic acid (TFA), triisopropyl silane (TIS) and water;
b) for producing the compound of formula I when M is not present, the yield of preparing the compound of formula I is at least 70%, preferably at least 80%, more preferably at least 90%, more preferably at least 95%, more preferably at least 99%; or
c) for producing the compound of formula I when M is present, wherein
i) step (a), step (b) and step (c) are conducted in one pot;
ii) the compound of formula I-A is photo cleaved at a wavelength about 300-400 nm; preferably at 365 nm; and/or
iii) the compound of formula I-A is chemically cleaved; preferably the compound of formula I-A is chemically cleaved by a mixture of trifluoroacetic acid (TFA), triisopropyl silane (TIS) and water;
d) for producing the compound of formula I when M is present, the yield of preparing the compound of formula I is at least 70%, preferably at least 80%, more preferably at least 90%, more preferably at least 95%, more preferably at least 99%; or
e) for producing the compound of formula II when M is not present, wherein
i) step (a) and step (b) are conducted in one pot;
ii) the compound of formula II-A is photo cleaved at a wavelength about 300-400 nm; preferably at 365 nm; and/or
iii) the compound of formula II-A is chemically cleaved; preferably the compound of formula II-A is chemically cleaved by a mixture of trifluoroacetic acid (TFA), triisopropyl silane (TIS) and water;
f) for producing the compound of formula II when M is not present, the yield of preparing the compound of formula I is at least 70%, preferably at least 80%, more preferably at least 90%, more preferably at least 95%, more preferably at least 99%; or
g) for producing the compound of formula II when M is present, wherein
i) step (a), step (b) and step (c) are conducted in one pot;
ii) the compound of formula II-A is photo cleaved at a wavelength about 300-400 nm; preferably at 365 nm; and/or
iii) the compound of formula I-A is chemically cleaved; preferably the compound of formula I-A is chemically cleaved by a mixture of trifluoroacetic acid (TFA), triisopropyl silane (TIS) and water; or
h) for producing the compound of formula II when M is present, the yield of preparing the compound of formula I is at least 70%, preferably at least 80%, more preferably at least 90%, more preferably at least 95%, more preferably at least 99%;
[F]
wherein
a) R₄ is C₁-C₈ alkyl; preferably, R₄ is C₁-C₆ alkyl; more preferably, R₄ is C₃-C₆ alkyl; more preferably R₄ is butyl; most preferably, R₄ is tert-butyl;
b) L contains at least two, three, four, five or six amino acids; preferably L contains at least three, four, five amino acids; more preferably, L contains at least three or four amino acids; more preferably, L contains three amino acids;
c) L contains 6-aminohexanoic acid (Aca), 3-amino-3-(2-nitrophenyl)propionic acid (Anp) and pyroglutamic acid;
d) A is a targeting moiety, wherein the targeting moiety is trastuzumab, bombesin, somatostatin or 2-[3-(1,3-dicarboxypropyl)ureido]pentanedioic acid (DUPA) or a derivative or fragment thereof;
e) A is activating group, wherein the activating group is to activate carboxylic acids, preferably, the activating group is Bis(pentafluorophenyl) carbonate or N-Hydroxysuccinimide; or
f) R₄ is OH or t-butyl;
[G] wherein
a) for producing the compound of formula I when M is present, and
b) for producing the compound of formula II when M is present,
wherein
1) the metal ion is added in the presence of a buffer solution;
2) the metal ion is a radioactive metal ion;
3) the temperature of step (c) is at room temperature;
4) the temperature of step (c) is around 40-90° C., preferably 50-90° C., more preferably 60-90° C., more preferably 70-90° C.; most preferably 80° C.; and/or
5) the pH of step (c) is 3-8; preferably 4-7, more preferably 4.5-6.5;
wherein the buffer solution is sodium acetate (NaOAc) or N-2-hydroxyethylpiperazine-N′-2-ethanesulfonic acid (HEPES);
[H]
wherein the metal ion is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb); preferably, the metal is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), or Scandium-43 (⁴³Sc); more preferably, the metal is Gallium-67 (Ga⁶⁷), Gallium-68 (Ga⁶⁸), Copper-62 (⁶²Cu), Copper-64 (⁶⁴Cu), or Copper-67 (⁶⁷Cu).

3. The process of claim 2 [A], wherein
[A]
i) the compound in step (a) contains a Fmoc protecting group and a terminal NH₂;
ii) the base used in steps b) and c) are the same base; preferably, the base is a non-nucleophilic base; more preferably, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases; more preferably, the base is DIEA;
iii) the coupling reagent is dicyclohexylcarbodiimide (DCC), ethyl(dimethylaminopropyl) carbodiimide (EDC), benzotriazole-1-yl-oxy-tris-pyrrolidino-phosphonium hexafluorophosphate (PyBOP), benzotriazol-1-yloxytris(dimethylamino)phosphonium hexafluorophosphate (BOP), or 2-(1H-Benzotriazole-1-yl)-1,1,3,3-tetramethyluronium hexafluorophosphate (HBTU);
iv) the solvent is dihydrolevoglucosenone, γ-valerolactone, or dimethylformamide (DMF);
v) the active ester synthesis reagent is N, N′-disuccinimidyl carbonate or N-Hydroxysuccinimide (NHS); preferably NHS;
vi) in step (c), m is 1-15, more preferably m is 1-10, more preferably, m is 1-5; most preferably m is 5;
vii) the solid support surface is a resin, preferably wherein the resin is a rink amide resin, a wang resin, an agarose resin, a tentagel resin or an ion-exchange resin, more preferably, the resin is a tentagel resin;
viii) steps (a)-(d) are conducted in one pot; and/or
ix) the yield of preparing the compound of formula I-A is at least 70%, preferably at least 80%, more preferably at least 90%, more preferably at least 95%, more preferably at least 99%; and/or
[B]
(a) the compound in step (a) has the structure: 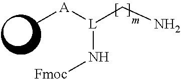
wherein m is 1-15, more preferably m is 1-10, more preferably, m is 1-5; most preferably m is 4;
(b) the compound in step (b) has the structure: 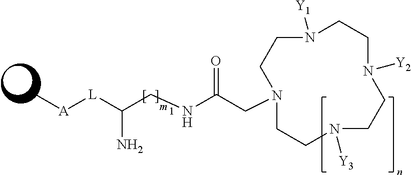
wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃; wherein m₁ is 1-15, more preferably m₁ is 1-10, more preferably, m₁ is 1-5; most preferably m₁ is 4;
(c) the compound in step (c) has the structure: 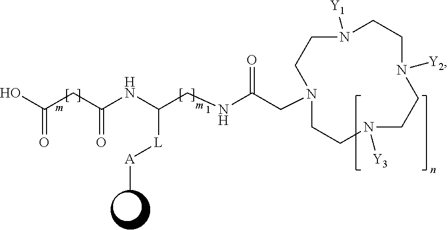
wherein m₁ is 1-15, more preferably m₁ is 1-10, more preferably, m₁ is 1-5; most preferably m₁ is 4; and/or
(d) the compound in step (d) has the structure

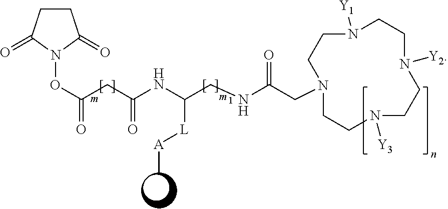

4. (canceled)

5. (canceled)

6. (canceled)

7. The process of claim 2[B], wherein
[A]
a) the base used in steps a), b) and c) is the same base; preferably, the base is a non-nucleophilic base; more preferably, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases; more preferably, the base is DIEA;
b) the deprotection reagent is a primary, secondary or tertiary amine; preferably a primary or secondary amine, more preferably a primary amine;
c) the acid containing a Fmoc protecting group in step (a) is 3-(Fmoc-amino)-3-(2-nitrophenyl) propionic acid, Fmoc-Asp-OH, Fmoc-Asn-OH, 3-(Fmoc-amino)-3-(2-nitrophenyl) propionic acid, Fmoc-8-Aoc-OH, Fmoc-5-Ava-Oh, Fmoc-p-Ala-OH, Fmoc-GABA-OH, Fmoc-11-Aun-OH, Fmoc-NH-(PEG)-COOH or Fmoc-6-Ahx-OH; preferably, the acid is 3-(Fmoc-amino)-3-(2-nitrophenyl) propionic acid;
d) the coupling reagent is dicyclohexylcarbodiimide (DCC), ethyl(dimethylaminopropyl) carbodiimide (EDC), benzotriazole-1-yl-oxy-tris-pyrrolidino-phosphonium hexafluorophosphate (PyBOP), benzotriazol-1-yloxytris(dimethylamino)phosphonium hexafluorophosphate (BOP), or 2-(1H-Benzotriazole-1-yl)-1,1,3,3-tetramethyluronium hexafluorophosphate (HBTU);
e) in step (d), R₄ is alkyl, alkenyl, alkynyl, alkyl-CF₃, preferably, R₄ is alkyl, alkenyl, alkynyl, more preferably, R₄ is alkyl, alkenyl, most preferably, R₄ is alkyl;
f) the solvent is dihydrolevoglucosenone, γ-valerolactone, or dimethylformamide (DMF);
g) the solid support surface is a resin, preferably, the resin is a rink amide resin, a wang resin, an agarose resin, a tentagel resin or an ion-exchange resin, more preferably the resin is a tentagel resin;
h) in step (b), A and L are coupled sequentially or coupled together; and/or
i) steps (a)-(d) are conducted in one pot;
[B]
a) the compound produced in step (b) has the following structure: 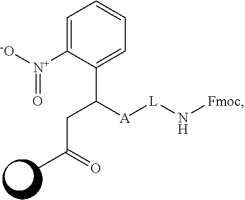
b) the acid containing a Fmoc in step (c) has the following structure: 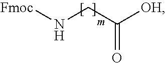
where in m is 1-20, more preferably m is 1-10, more preferably, m is 1-5; most preferably m is 5; and/or
c) the compound produced in step (c) has the following structure: 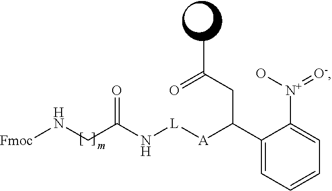
and/or
[C]
wherein the acid containing a Fmoc protecting group in step (a) is Fmoc-Trp(Boc)-OH, Fmoc-Asp-OH, Fmoc-Asn-OH, 3-(Fmoc-amino)-3-(2-nitrophenyl) propionic acid, Fmoc-8-Aoc-OH, Fmoc-5-Ava-Oh, Fmoc-β-Ala-OH, Fmoc-GABA-OH, Fmoc-11-Aun-OH, Fmoc-NH-(PEG)-COOH or Fmoc-6-Ahx-OH; preferably, the acid is Fmoc-Trp (Boc)-OH.

8. The process of claim 7, wherein
a) the deprotection reagent is cyclohexylamine, ethanolamine, piperidine, piperazine; more preferably piperidine;
b) the concentration of piperidine is about 10-50%, preferably 10-40%, more preferably 10-30%, most preferably 20%; and/or
c) R₄ is C₁₋₈ alkyl, preferably, R₄ is C₁₋₆ alkyl, more preferably R₄ is C₂₋₅ alkyl; more preferably R₄ is butyl; most preferably R₄ is tert-butyl.

9. (canceled)

10. (canceled)

11. (canceled)

12. (canceled)

13. The process of claim 2[C], wherein
a) the base used in steps a) and b) is the same base; preferably, the base is a non-nucleophilic base; more preferably, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases; more preferably, the base is DIEA;
b) the active ester synthesis reagent is N, N′-disuccinimidyl carbonate or N-Hydroxysuccinimide (NHS); preferably NHS;
c) the coupling reagent is dicyclohexylcarbodiimide (DCC), ethyl(dimethylaminopropyl) carbodiimide (EDC), benzotriazole-1-yl-oxy-tris-pyrrolidino-phosphonium hexafluorophosphate (PyBOP), benzotriazol-1-yloxytris(dimethylamino)phosphonium hexafluorophosphate (BOP), or 2-(1H-Benzotriazole-1-yl)-1,1,3,3-tetramethyluronium hexafluorophosphate (HBTU); preferably EDC;
d) the solid support surface is a resin, preferably, the resin is a rink amide resin, a wang resin, an agarose resin, a tentagel resin or an ion-exchange resin, preferably, the resin is a wang resin;
e) the solvent in step (b) is dihydrolevoglucosenone, γ-valerolactone or dimethylformamide (DMF);
f) step (b) is performed at a temperate of 30-80° C., preferably, 40-60° C., more preferably 50° C.;
g) steps (a)-(b) are conducted in one pot; and/or
h) the yield of preparing the compound of formula II-A is at least 70%, more preferably at least 80%, more preferably at least 90%, more preferably at least 95%, more preferably at least 99%.

14. (canceled)

15. The process of claim 2, wherein L contains amino acids selected from serine (ser/S), glycine (gly/G), tryptophan (trp/W), glutamine (gln/Q), arginine (arg/R), leucine (leu/L), asparagine (asn/N), alanine (ala/A), valine (val/V), histidine (his/H), and methionine (met/M); preferably, L contains amino acids selected from Q, W and G; more preferably, L contains amino acids selected from Q and G.

16. The process of claim 15, wherein L contains
a) Aca-gln-trp;
b) gln-trp-ala-val-gly-his-leu-met;
c) gly-gly-trp;
d) Aca-bombesin (BBN)
e) ser-gly-trp; or
f) Aca-gln-trp-anp.

17. (canceled)

18. The process of claim 2[D] wherein
a) the reaction is conducted in base; preferably, the base is a non-nucleophilic base; more preferably, the base is N, N-Diisopropylethylamine (DIEA), 1,8-Diazabicycloundec-7-sene (DBU), 1,5-Diazabicyclo(4.3.0)non-5-ene (DBN), 2,6-Di-tert-butylpyridine, or phosphazene bases; more preferably, the base is DIEA; and/or
b) the reaction is conducted at a temperate of 30-80° C., preferably, 40-60° C., more preferably 50° C. or
the process further comprising the following steps for producing the compound of formula IV:
a) coupling an acid containing a fluorenylmethoxycarbonyl protecting group (Fmoc) using a coupling reagent in the presence of a base to a solid support surface in a solvent;
b) deprotecting the Fmoc group with a deprotection reagent in the presence of a solvent and coupling groups L to the acid using a coupling reagent in the presence of a base;
c) coupling an acid containing a Fmoc protection group using a coupling reagent in the presence of a base and a solvent to the compound obtained in step (b); and
d) deprotecting the Fmoc group with a deprotection reagent and coupling the compound obtained in step (c) with the compound of formula IV-A in a solvent: 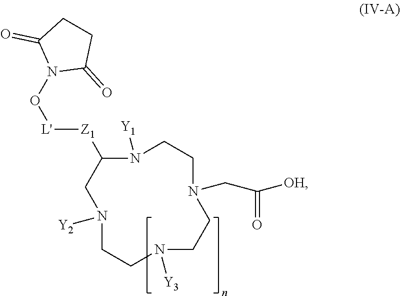
wherein Y₁, Y₂, Y₃ are each, independently, alkyl-CO₂R₄, wherein R₄ is H, alkyl, alkenyl, alkynyl, alkyl-aryl, alkyl-heteroaryl, aryl, heteroaryl, alkyl-CF₃.

19. (canceled)

20. The process of claim 1, wherein the compound of formula I has the following structure: 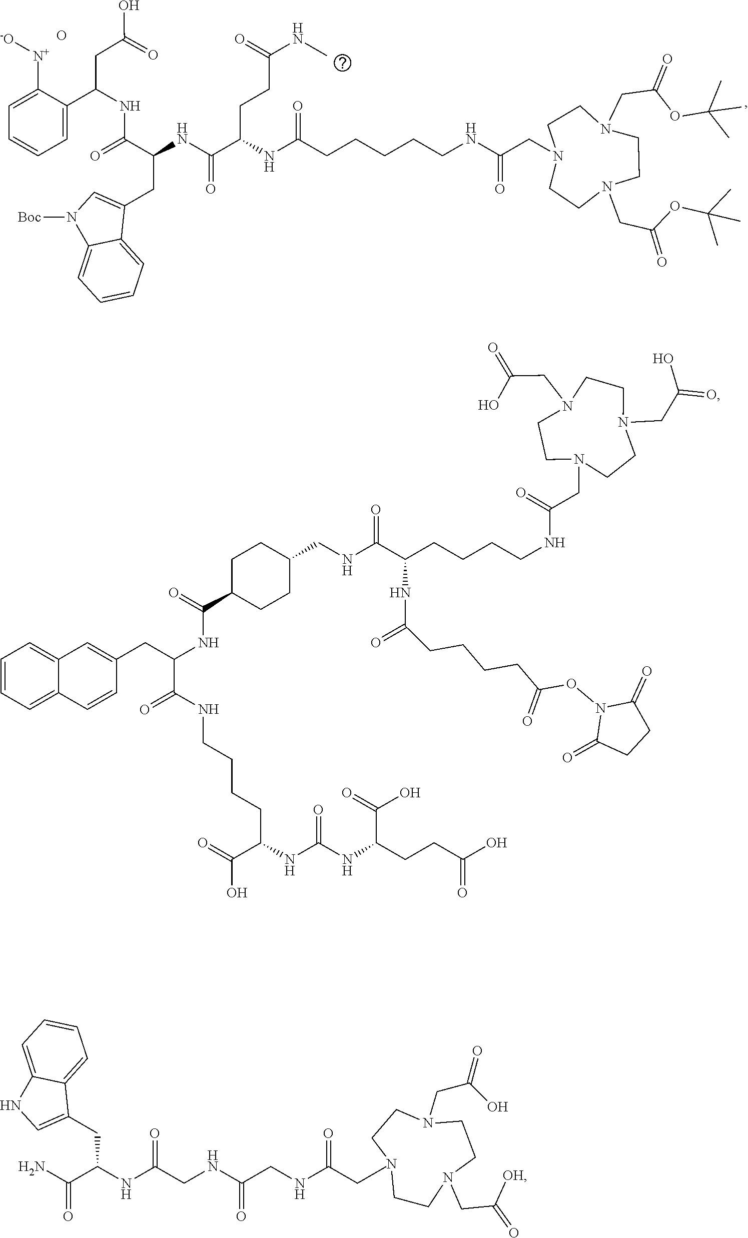 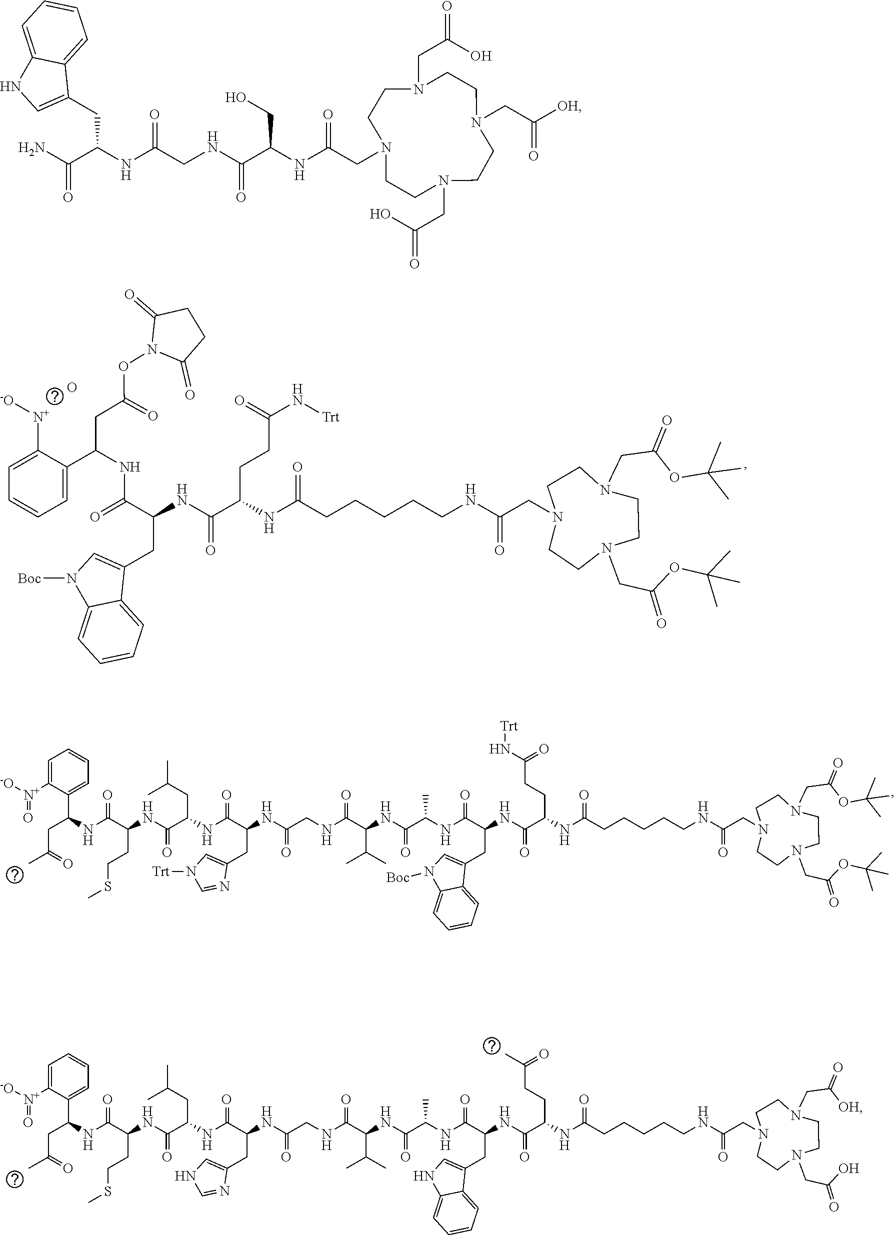 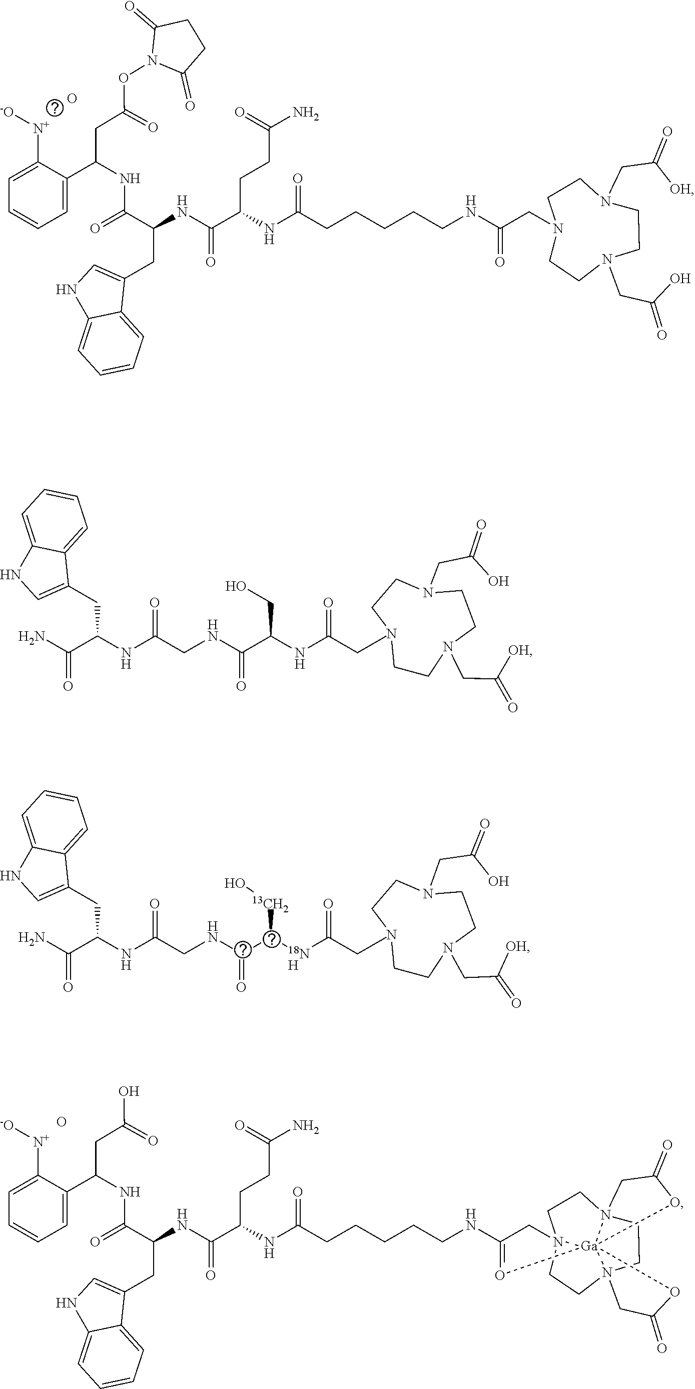 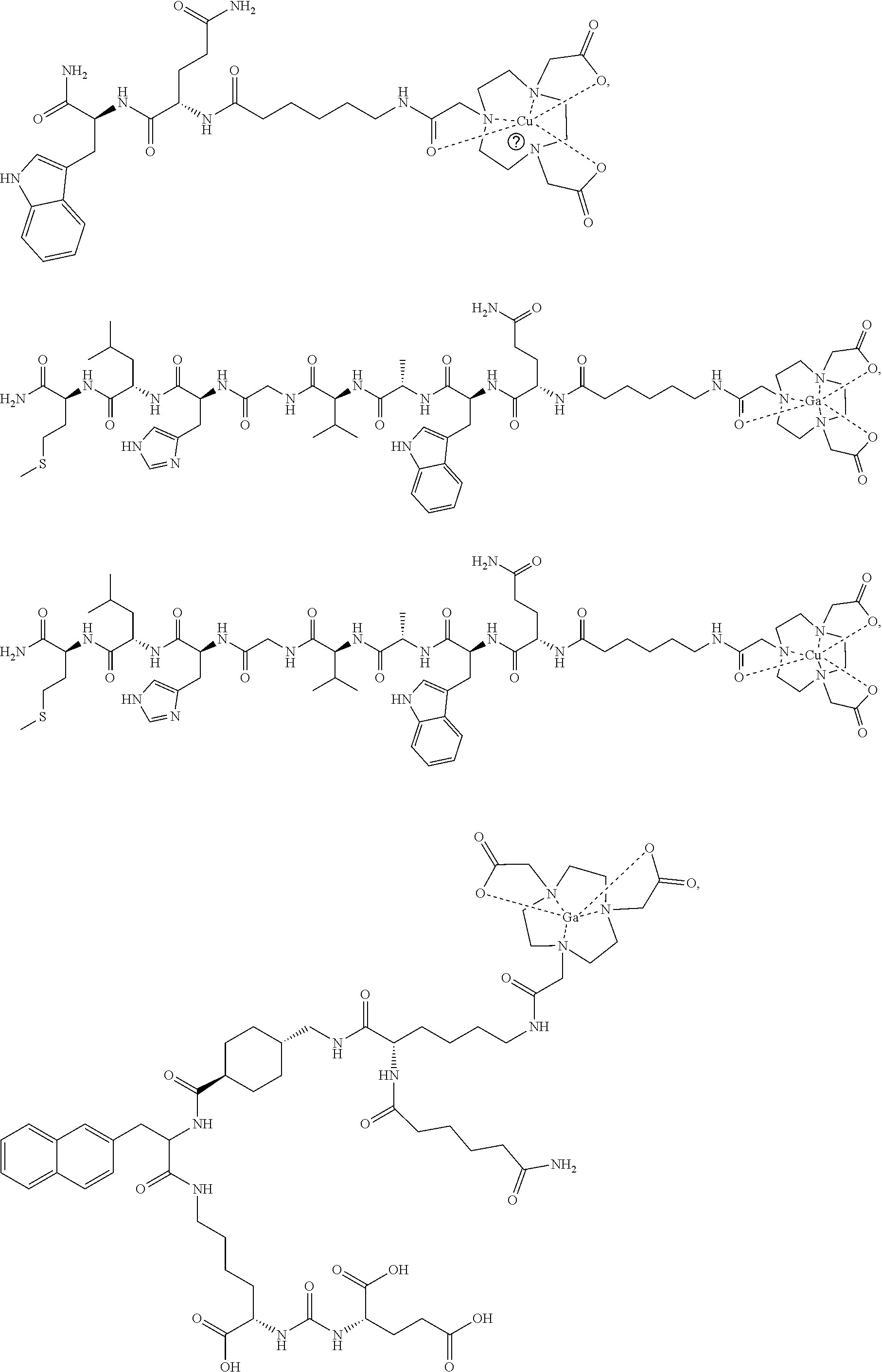 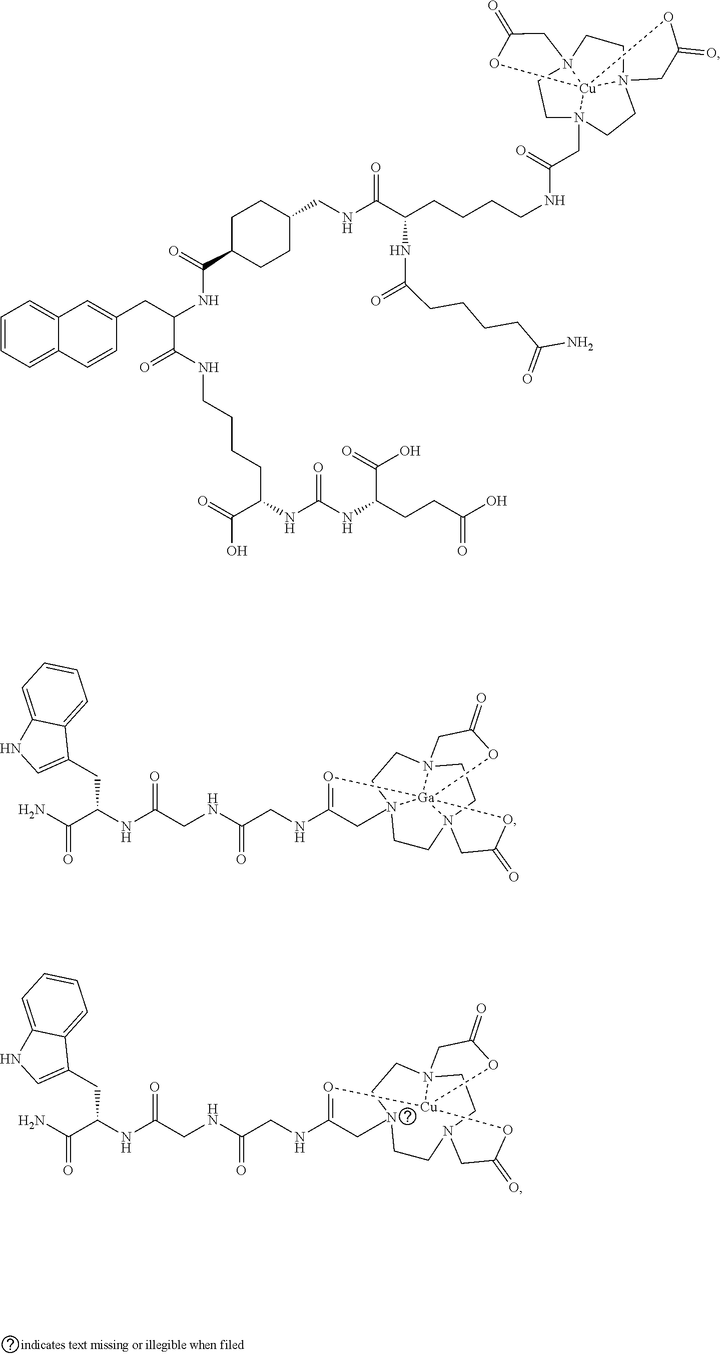 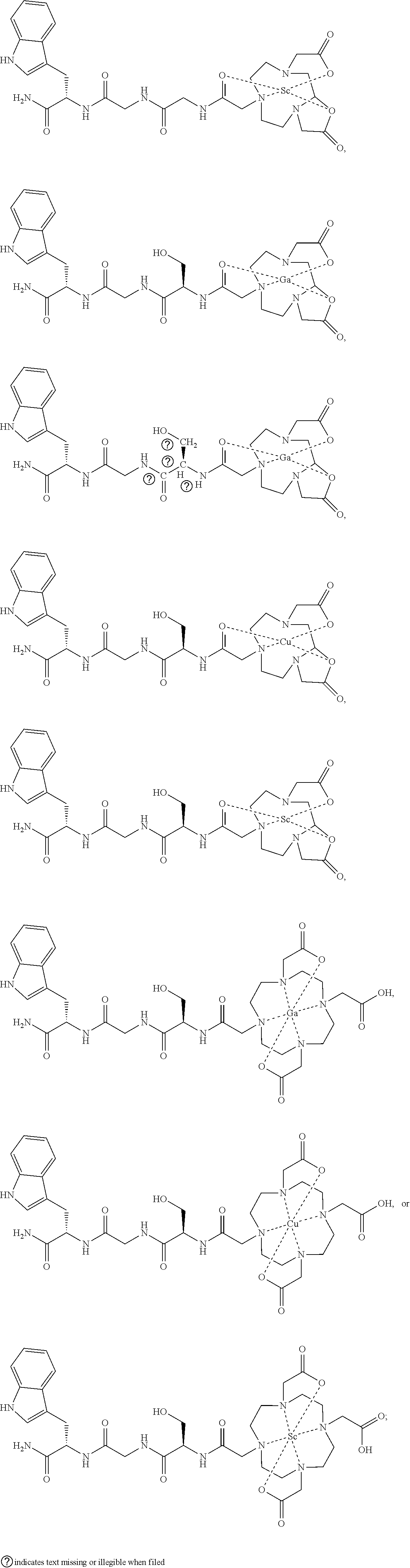
or wherein the compound of formula II has the following structure:

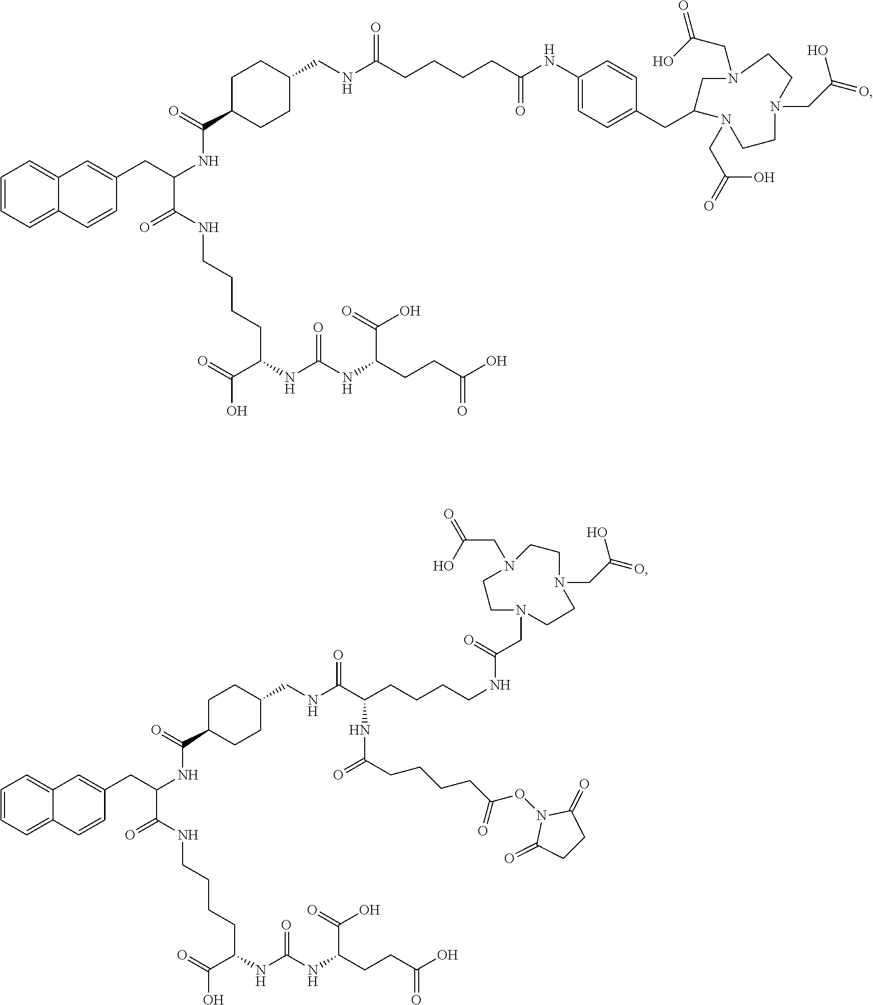 [SMILES: O=C(O)CC[C@H](NC(=O)N[C@@H](CCCCNC(=O)C(Cc1ccc2ccccc2c1)NC(=O)[C@H]1CC[C@H](CNC(=O)CCCCC(=O)Nc2ccc(CC3CN(CC(=O)O)CCN(CC(=O)O)CCN3CC(=O)O)cc2)CC1)C(=O)O)C(=O)O ; O=C(O)CC[C@H](NC(=O)N[C@@H](CCCCNC(=O)C(Cc1ccc2ccccc2c1)NC(=O)[C@H]1CC[C@H](CNC(=O)[C@H](CCCCNC(=O)CN2CCN(CC(=O)O)CCN(CC(=O)O)CC2)NC(=O)CCCCC(=O)ON2C(=O)CCC2=O)CC1)C(=O)O)C(=O)O]

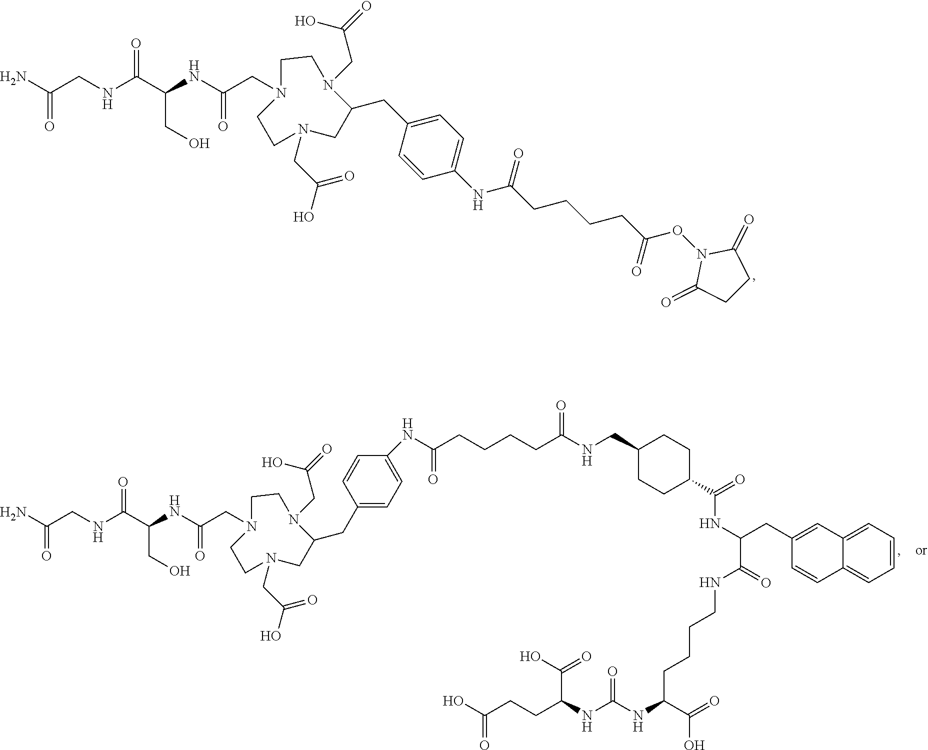 [SMILES: NC(=O)CNC(=O)[C@H](CO)NC(=O)CN1CCN(CC(=O)O)CC(Cc2ccc(NC(=O)CCCCC(=O)ON3C(=O)CCC3=O)cc2)N(CC(=O)O)CC1 ; NC(=O)CNC(=O)[C@H](CO)NC(=O)CN1CCN(CC(=O)O)CC(Cc2ccc(NC(=O)CCCCC(=O)NC[C@H]3CC[C@H](C(=O)NC(Cc4ccc5ccccc5c4)C(=O)NCCCC[C@H](NC(=O)N[C@@H](CCC(=O)O)C(=O)O)C(=O)O)CC3)cc2)N(CC(=O)O)CC1]

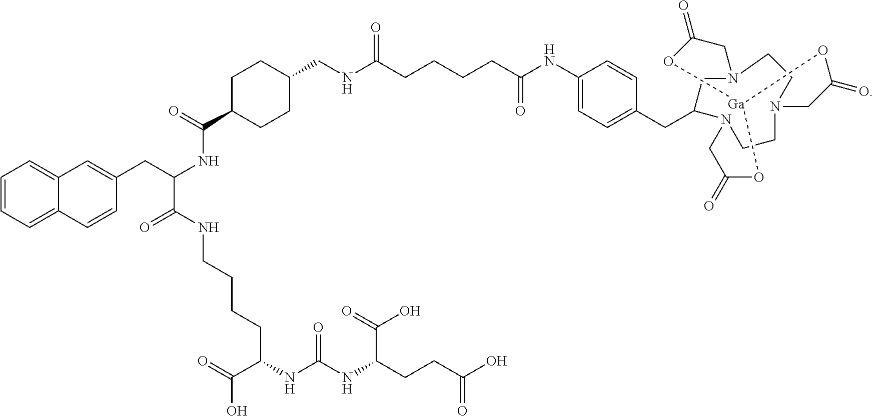

21. (canceled)

22. (canceled)

23. (canceled)

24. The process of claim 2[G], wherein
a) the buffer solution is sodium acetate (NaOAc) or N-2-hydroxyethylpiperazine-N′-2-ethanesulfonic acid (HEPES); and/or
b) the pH of the metal ion solution is around 4-6, preferably 5.

25. (canceled)

26. (canceled)

27. (canceled)

28. (canceled)

29. A composition comprising a compound attached to a solid surface having the structure: 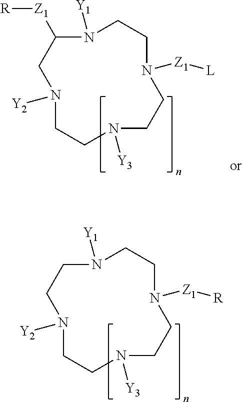
wherein
wherein R is 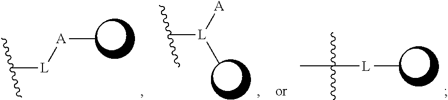
n is 0 or 1;
Y₁, Y₂, Y₃ are each, independently, —H, alkylheteroaryl, alkyl-CO₂H, alkylaryl-CO₂H, alkylheteroaryl-CO₂H, alkyl-CO₂R₄, alkylaryl-NH—CO₂R₄, alkylaryl-CO₂R₄, alkylheteroaryl-CO₂R₄, alkyl-OH, alkylaryl-OH, alkylheteroaryl-OH, alkyl-N(alkylaryl)₂, alkyl-N(alkylaryl-CO₂H)₂, alkyl-N(alkylheteroaryl-CO₂H)₂, alkyl-N (alkylaryl-CO₂R₄)₂, alkyl-N(alkylheteroaryl-CO₂R₄)₂, alkyl-N(alkylaryl-OH)₂, alkyl-N(alkylheteroaryl-OH)₂, alkyl-N(alkyl-CO₂H)₂, alkyl-N(alkylaryl-OH)(alkyl-CO₂H), alkyl-N(alkylheteroaryl-OH)(alkyl-CO₂H), alkyl-P(O)(OH)₂, alkylaryl-P(O)(OH)₂ and alkylheteroaryl-P(O)(OH)₂;
wherein each occurrence of R₄ is independently, —H, alkyl, alkenyl, alkynyl, alkyl-aryl, alkyl-heteroaryl, aryl, heteroaryl, alkyl-CF₃ or —Si(alkyl)₃;
Z₁ is 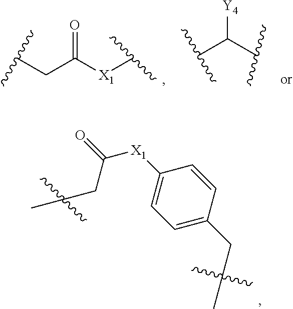
wherein X₁ is NH, O or S, and
Y₄ is —CO₂H, —CO₂R₅, aryl-CO₂H, heteroaryl-CO₂H, aryl-CO₂R₅ or heteroaryl-CO₂R₅, and
wherein each occurrence of R₅ is, independently, —H, alkyl, alkenyl, alkynyl, alkyl-aryl, alkyl-heteroaryl, aryl, heteroaryl, alkyl-CF₃ or —Si(alkyl)₃;
L is a peptide linker;
A is a targeting moiety or an activating group;
or a pharmaceutically acceptable salt of the compound.

30. The composition of claim 29, wherein
(a) one of Y₁ or Y₂ is —H, or
each of Y₁ and Y₂ is —H; or
one of Y₁ or Y₂ is alkyl-CO₂H, or
each of Y₁ and Y₂ is alkyl-CO₂H; or
one of Y₁ or Y₂ is alkylaryl-CO₂H or alkyl-CO₂R₄, or
each of Y₁ and Y₂ are alkylaryl-CO₂H or alkyl-CO₂R₄;
preferably, R₄ is alkyl, alkenyl, alkynyl; more preferably, R₄ is alkyl, alkenyl; more preferably, R₄ is alkyl; more preferably, R₄ is C₁-C₈ alkyl; more preferably, R₄ is C₁-C₆ alkyl; more preferably, R₄ is C₃-C₆ alkyl; more preferably R₄ is butyl; most preferably, R₄ is tert-butyl;
(b) L contains at least two, three, four, five or six amino acids; preferably L contains at least three, four, five amino acids; more preferably, L contains at least three or four amino acids; more preferably, L contains three amino acids; more preferably, L contains 6-aminohexanoic acid (Aca), 3-amino-3-(2-nitrophenyl)propionic acid (Anp) and pyroglutamic acid; preferably, L contains amino acids selected from serine (ser/S), glycine (gly/G), tryptophan (trp/W), glutamine (gln/Q), arginine (arg/R), leucine (leu/L), asparagine (asn/N), alanine (ala/A), valine (val/V), histidine (his/H), and methionine (met/M); preferably, L contains amino acids selected from Q, W and G; more preferably, L contains amino acids selected from Q and G;
(c) A is a targeting moiety, wherein the targeting moiety is trastuzumab, bombesin, somatostatin or 2-[3-(1,3-dicarboxypropyl)ureido]pentanedioic acid (DUPA) or a derivative or fragment thereof, or wherein A is activating group, wherein the activating group is to activate carboxylic acids, preferably, the activating group is Bis(pentafluorophenyl) carbonate or N-Hydroxysuccinimide.

31. (canceled)

32. (canceled)

33. (canceled)

34. (canceled)

35. (canceled)

36. The composition of claim 35, wherein
L is
a) Aca-gln-trp;
b) gln-trp-ala-val-gly-his-leu-met;
c) gly-gly-trp;
d) Aca-bombesin (BBN)
e) ser-gly-trp; or
f) Aca-gln-trp-anp.

37. (canceled)

38. (canceled)

39. The composition of claim 29 having the structure:

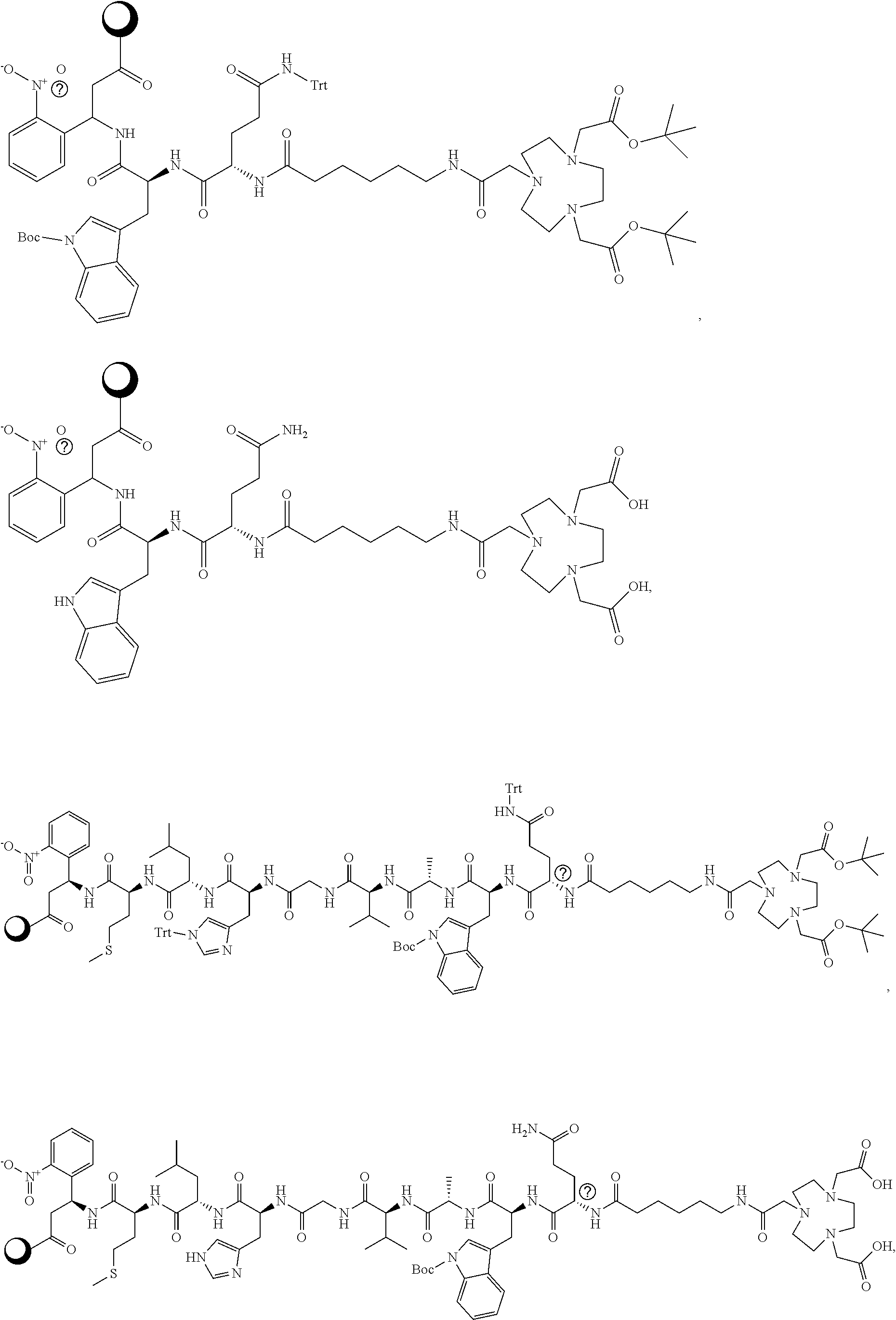

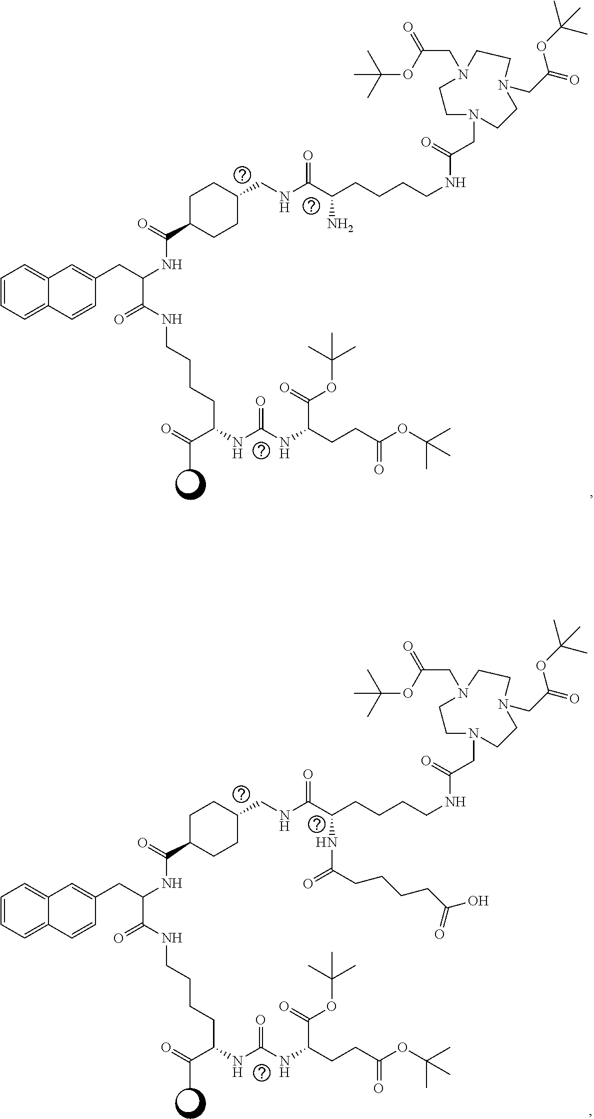 [SMILES: CC(=O)[C@H](CCCCNC(=O)C(Cc1ccc2ccccc2c1)NC(=O)[C@H]1CC[C@H](CNC(=O)[C@@H](N)CCCCNC(=O)CN2CCN(CC(=O)OC(C)(C)C)CCN(CC(=O)OC(C)(C)C)CC2)CC1)NC(=O)N[C@@H](CCC(=O)OC(C)(C)C)C(=O)OC(C)(C)C ; CC(=O)[C@H](CCCCNC(=O)C(Cc1ccc2ccccc2c1)NC(=O)[C@H]1CC[C@H](CNC(=O)[C@H](CCCCNC(=O)CN2CCN(CC(=O)OC(C)(C)C)CCN(CC(=O)OC(C)(C)C)CC2)NC(=O)CCCCC(=O)O)CC1)NC(=O)N[C@@H](CCC(=O)OC(C)(C)C)C(=O)OC(C)(C)C]

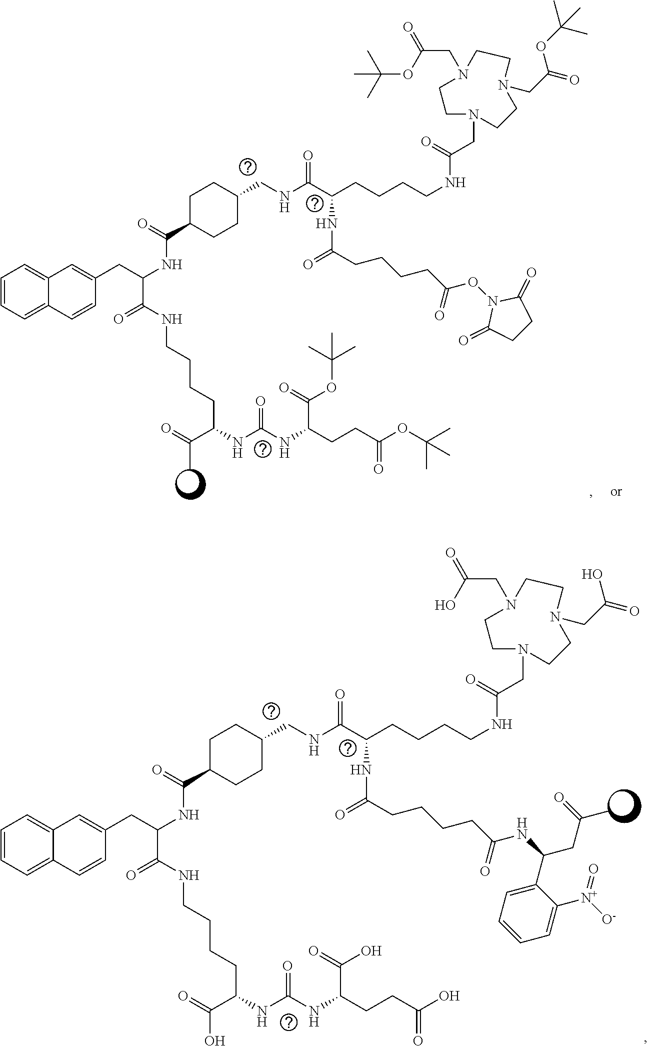 [SMILES: CC(=O)[C@H](CCCCNC(=O)C(Cc1ccc2ccccc2c1)NC(=O)[C@H]1CC[C@H](CNC(=O)[C@H](CCCCNC(=O)CN2CCN(CC(=O)OC(C)(C)C)CCN(CC(=O)OC(C)(C)C)CC2)NC(=O)CCCCC(=O)ON2C(=O)CCC2=O)CC1)NC(=O)N[C@@H](CCC(=O)OC(C)(C)C)C(=O)OC(C)(C)C ; CC(=O)C[C@H](NC(=O)CCCCC(=O)N[C@@H](CCCCNC(=O)CN1CCN(CC(=O)O)CCN(CC(=O)O)CC1)C(=O)NC[C@H]1CC[C@H](C(=O)NC(Cc2ccc3ccccc3c2)C(=O)NCCCC[C@H](NC(=O)N[C@@H](CCC(=O)O)C(=O)O)C(=O)O)CC1)c1ccccc1[N+](=O)[O-]]

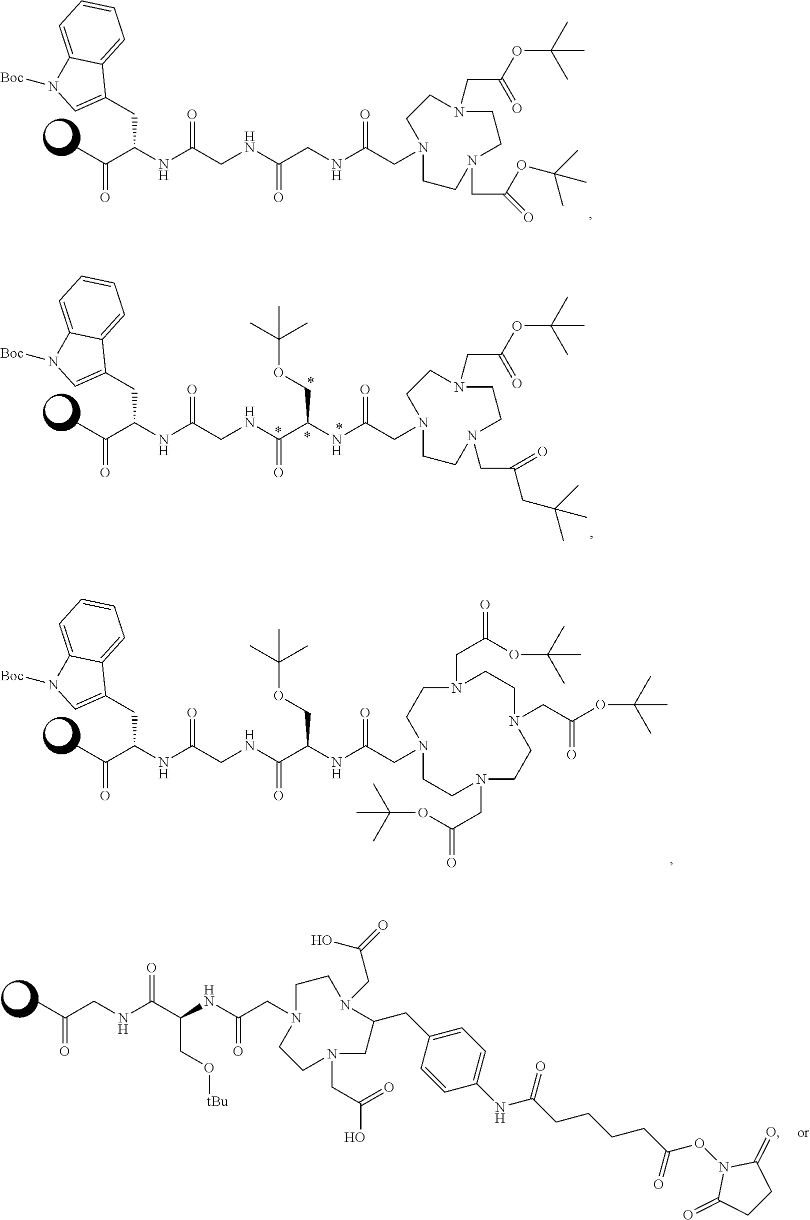

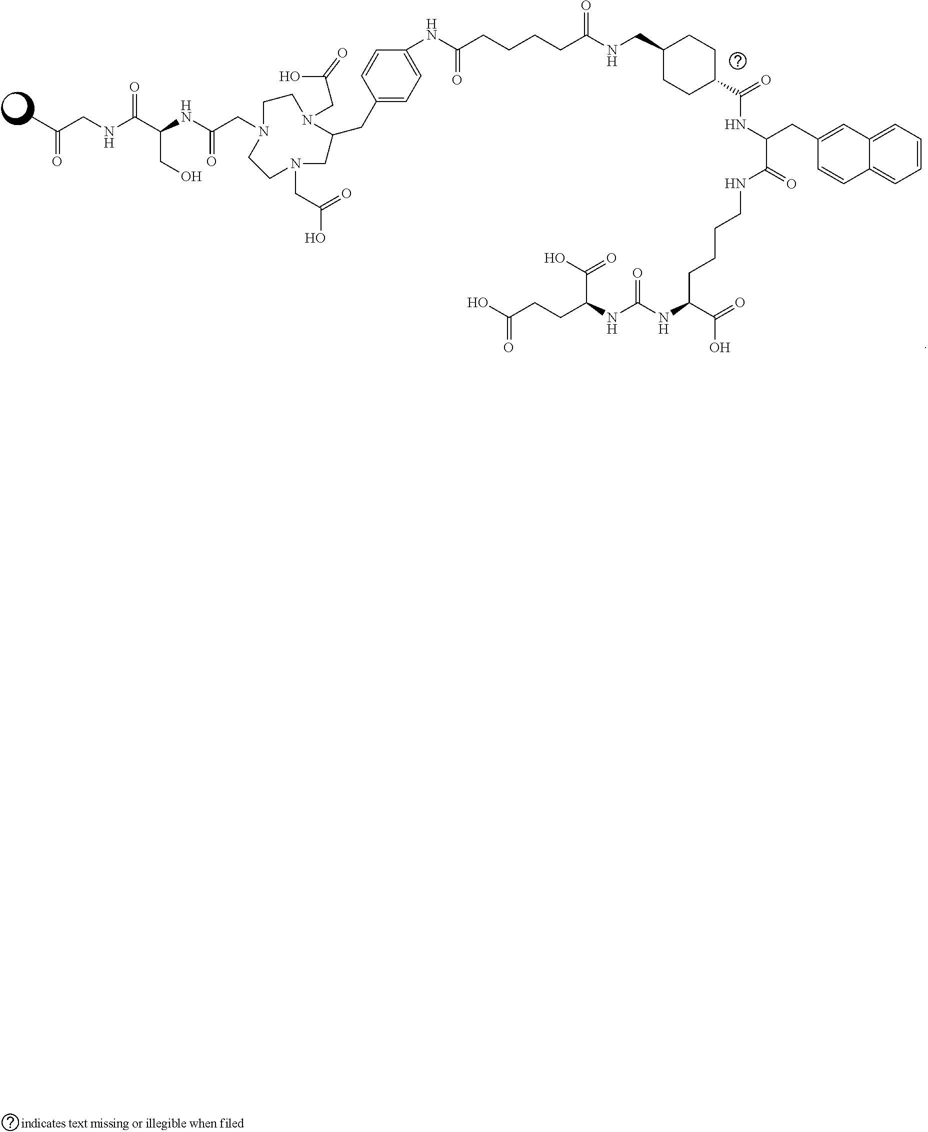 [SMILES: CC(=O)CNC(=O)[C@H](CO)NC(=O)CN1CCN(CC(=O)O)CC(Cc2ccc(NC(=O)CCCCC(=O)NC[C@H]3CC[C@H](C(=O)NC(Cc4ccc5ccccc5c4)C(=O)NCCCC[C@H](NC(=O)N[C@@H](CCC(=O)O)C(=O)O)C(=O)O)CC3)cc2)N(CC(=O)O)CC1]

40. (canceled)

41. The composition of claim 29, wherein the composition further coordinates with a metal ion to form a metal complex; preferably the metal ion is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), Scandium-43 (⁴³Sc), Lanthanum-132 (¹³²La), Lanthanum-135 (¹³⁵La), Yttrium-86 (⁸⁶Y), Yttrium-90 (⁹⁰Y), Lutetium 177 (¹⁷⁷Lu), Terbium-149 (¹⁴⁹Tb), Terbium-152 (¹⁵²Tb), Terbium-155 (¹⁵⁵Tb) or Terbium-161 (¹⁶¹Tb); preferably, the metal is Gallium-67 (Ga⁶⁷), Gallium-68 (Ga⁶⁸), Copper-62 (⁶²Cu), Copper-64 (⁶⁴Cu), Copper-67 (⁶⁷Cu), Scandium-44 (⁴⁴Sc), Scandium-47 (⁴⁷Sc), or Scandium-43 (⁴³Sc) more preferably, the metal is Gallium-67 (Ga⁶⁷), Gallium-68 (Ga⁶⁸), Copper-62 (⁶²Cu), Copper-64 (⁶⁴Cu), or Copper-67 (⁶⁷Cu).

42. (canceled)

43. (canceled)

44. (canceled)

45. A method of imaging target cells in a subject comprising:
a) administering to the subject an effective amount of the metal complex of claim 41 or a pharmaceutically acceptable salt thereof, wherein the metal complex or the composition specifically accumulates at the target cells in the subject;
b) detecting in the subject the location of the metal complex or the composition; and
c) obtaining an image of the target cells in the subject based on the location of the metal complex or the composition in the subject.

46. A method of detecting the presence of target cells in a subject which comprises determining if an amount of the metal complex of claim 41 or a pharmaceutically acceptable salt thereof, is present in the subject at a period of time after administration of the metal complex or composition to the subject, thereby detecting the presence of the target cells based on the amount of the metal complex or composition determined to be present in the subject.

47. The method of claim 45, wherein
(a) the detection is performed by a Positron Emission Tomography (PET) device;
(b) the target cells are cancer cells; preferably, the cancer cells are prostate cancer cells, wherein the cancer cells have elevated levels of prostate-specific membrane antigen (PSMA); and/or
(c) the method comprises:
a) conjugate the metal complex to a solid support surface;
b) transport the metal complex and the solid support surface to an imaging device; and
c) photocleave the metal complex; preferably, the metal complex is photo cleaved at a wavelength about 300-400 nm;
preferably at 320-370 nm; more preferably at 365 nm.

48. (canceled)

49. (canceled)

50. (canceled)

51. (canceled)

52. (canceled)

53. A method of reducing the size of a prostate tumor or of inhibiting proliferation of prostate cancer cells comprising contacting the tumor or cancer cells with the metal complex of claim 41 or a pharmaceutically acceptable salt thereof, so as to thereby reducing the size of the tumor or inhibit proliferation of the cancer cells.
